SEQUENCE LISTING (78 sequences)

SEQ ID NO: 1 - DNA, 1509 bp, Homo sapiens
  misc_feature (1)..(1509): FL-WASP
atgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactga

SEQ ID NO: 2 - protein, 502 aa, Homo sapiens
  misc_feature (1)..(502): FL-WASP
MSGGPMGGRPGGRGAPAVQQNIPSTLLQDHENQRLFEMLGRKCLTLATAVVQLYLALPPGAEHWTKEHCGAVCFVKDNPQKSYFIRLYGLQAGRLLWEQELYSQLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 3 - DNA, 1518 bp, Homo sapiens
  misc_feature (1)..(1518): FL-N-WASP
atgagctccgtccagcagcagccgccgccgccgcggagggtcaccaacgtggggtccctgttgctcaccccgcaggagaacgagtccctcttcactttcctcggcaagaaatgtgtgactatgtcttcagcagtggtgcagttatatgcagcagatcggaactgtatgtggtcaaagaagtgcagtggtgttgcttgtcttgttaaggacaatccacagagatctcattttttaagaatatttgacattaaggatgggaaactattgtgggaacaagagctatacaataactttgtatataatagtcctagaggatattttcatacctttgctggagatacttgtcaagttgctcttaattttgccaatgaagaagaagcaaaaaaatttcgaaaagcagttacagaccttttgggccgtcgacaaaggaaatctgagaaaagacgagatcccccaaatggtcctaatctacccatggctacagttgatataaaaaatccagaaatcacaacaaatagattttatggtccacaagtcaacaacatctcccataccaaagaaaagaagaagggaaaagctaaaaagaagagattaaccaagggagatataggaacaccaagcaatttccagcacattggacatgttggttgggatccaaatacaggctctgatctgaataatttggatccagaattgaagaatctttttgatatgtgtggaatcttagaggcacaacttaaagaaagagaaacattaaaagttatatatgactttattgaaaaaacaggaggtgttgaagctgttaaaaatgaactgcggaggcaagcaccaccacctccaccaccatcaaggggagggccacctcctcctcctccccctccacatagctcgggtcctcctcctcctcctgctaggggaagaggcgctcctcccccaccaccttcaagagctcccacagctgcacctccaccaccgcctccttccaggccaagtgtagaagtccctccaccaccgccaaataggatgtaccctcctccacctccagcccttccctcctcagcaccttcagggcctccaccaccacctccatctgtgttgggggtagggccagtggcaccacccccaccgcctccacctccacctcctcctgggccaccgcccccgcctggcctgccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagactga

SEQ ID NO: 4 - protein, 505 aa, Homo sapiens
  misc_feature (1)..(505): FL-N-WASP
MSSVQQQPPPPRRVTNVGSLLLTPQENESLFTFLGKKCVTMSSAVVQLYAADRNCMWSKKCSGVACLVKDNPQRSYFLRIFDIKDGKLLWEQELYNNFVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKADIGTPSNFQHIGHVGWDPNTGFDLNNLDPELKNLFDMCGISEAQLKDRETSKVIYDFIEKTGGVEAVKNELRRQAPPPPPPSRGGPPPPPPPPHNSGPPPPPARGRGAPPPPPSRAPTAAPPPPPPSRPSVAVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWED

SEQ ID NO: 5 - DNA, 225 bp, Homo sapiens
  misc_feature (1)..(225): WASP VCA Domain
gggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactga

SEQ ID NO: 6 - protein, 74 aa, Homo sapiens
  misc_feature (1)..(74): WASP VCA Domain
GGRGALLDQIRQGIQLNKTPGAPESSALQPPPQSSEGLVGALMHVMQKRSRAIHSSDEGEDQAGDEDEDDEWDD

SEQ ID NO: 7 - DNA, 345 bp, Homo sapiens
  misc_feature (1)..(345): N-WASP VCA Domain
ccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagactag

SEQ ID NO: 8 - protein, 114 aa, Homo sapiens
  misc_feature (1)..(114): N-WASP VCA Domain
PSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWED

SEQ ID NO: 9 - DNA, 1197 bp, Homo sapiens
  misc_feature (1)..(1197): 105WASP
cttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactag

SEQ ID NO: 10 - protein, 398 aa, Homo sapiens
  misc_feature (1)..(398): 105WASP
LVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 11 - DNA, 1227 bp, Homo sapiens
  misc_feature (1)..(1227): 98N-WASP
tttgtatataatagtcctagaggatattttcatacctttgctggagatacttgtcaagttgctcttaattttgccaatgaagaagaagcaaaaaaatttcgaaaagcagttacagaccttttgggccgtcgacaaaggaaatctgagaaaagacgagatcccccaaatggtcctaatctacccatggctacagttgatataaaaaatccagaaatcacaacaaatagattttatggtccacaagtcaacaacatctcccataccaaagaaaagaagaagggaaaagctaaaaagaagagattaaccaagggagatataggaacaccaagcaatttccagcacattggacatgttggttgggatccaaatacaggctctgatctgaataatttggatccagaattgaagaatctttttgatatgtgtggaatcttagaggcacaacttaaagaaagagaaacattaaaagttatatatgactttattgaaaaaacaggaggtgttgaagctgttaaaaatgaactgcggaggcaagcaccaccacctccaccaccatcaaggggagggccacctcctcctcctccccctccacatagctcgggtcctcctcctcctcctgctaggggaagaggcgctcctcccccaccaccttcaagagctcccacagctgcacctccaccaccgcctccttccaggccaagtgtagaagtccctccaccaccgccaaataggatgtaccctcctccacctccagcccttccctcctcagcaccttcagggcctccaccaccacctccatctgtgttgggggtagggccagtggcaccacccccaccgcctccacctccacctcctcctgggccaccgcccccgcctggcctgccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagactag

SEQ ID NO: 12 - protein, 408 aa, Homo sapiens
FVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKADIGTPSNFQHIGHVGWDPNTGFDLNNLDPELKNLFDMCGISEAQLKDRETSKVIYDFIEKTGGVEAVKNELRRQAPPPPPPSRGGPPPPPPPPHNSGPPPPPARGRGAPPPPPSRAPTAAPPPPPPSRPSVAVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWED

SEQ ID NO: 13 - DNA, 2148 bp, Artificial
  Nucleotide sequence encoding Myc-WASP-TAP fusion protein
atgggagagcagaaactgatctctgaagaagacctgaacgatccatcacactggcggccgcagatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgacgagcggccgctcgagaccatggaaaagagaagatggaaaaagaatttcatagccgtctcagcagccaaccgctttaagaaaatctcatcctccggggcacttgattatgatattccaactactgctagcgagaatttgtattttcagggtgagctcaaaaccgcggctcttgcgcaacacgatgaagccgtggacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatggtgctcaggcgccgaaagtagacgcgaattccgcggggaagtcaaccggatccatctag

SEQ ID NO: 14 - protein, 715 aa, Artificial
  Myc-WASP-TAP fusion protein
MGEQKLISEEDLNDPSHWRPQMSGGPMGGRPGGRGAPAVQQNIPSTLLQDHENQRLFEMLGRKCLTLATAVVQLYLALPPGAEHWTKEHCGAVCFVKDNPQKSYFIRLYGLQAGRLLWEQELYSQLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDDERPLETMEKRRWKKNFIAVSAANRFKKISSSGALDYDIPTTASENLYFQGELKTAALAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPKVDANSAGKSTGSI

SEQ ID NO: 15 - DNA, 2151 bp, Artificial
  Nucleotide sequence encoding Myc-N-WASP-TAP fusion protein
atgggagagcagaaactgatctctgaagaagacctgaacgatccatcacactggcggccgctcgagatgagctccgtccagcagcagccgccgccgccgcggagggtcaccaacgtggggtccctgttgctcaccccgcaggagaacgagtccctcttcactttcctcggcaagaaatgtgtgactatgtcttcagcagtggtgcagttatatgcagcagatcggaactgtatgtggtcaaagaagtgcagtggtgttgcttgtcttgttaaggacaatccacagagatctcattttttaagaatatttgacattaaggatgggaaactattgtgggaacaagagctatacaataactttgtatataatagtcctagaggatattttcatacctttgctggagatacttgtcaagttgctcttaattttgccaatgaagaagaagcaaaaaaatttcgaaaagcagttacagaccttttgggccgtcgacaaaggaaatctgagaaaagacgagatcccccaaatggtcctaatctacccatggctacagttgatataaaaaatccagaaatcacaacaaatagattttatggtccacaagtcaacaacatctcccataccaaagaaaagaagaagggaaaagctaaaaagaagagattaaccaagggagatataggaacaccaagcaatttccagcacattggacatgttggttgggatccaaatacaggctctgatctgaataatttggatccagaattgaagaatctttttgatatgtgtggaatcttagaggcacaacttaaagaaagagaaacattaaaagttatatatgactttattgaaaaaacaggaggtgttgaagctgttaaaaatgaactgcggaggcaagcaccaccacctccaccaccatcaaggggagggccacctcctcctcctccccctccacatagctcgggtcctcctcctcctcctgctaggggaagaggcgctcctcccccaccaccttcaagagctcccacagctgcacctccaccaccgcctccttccaggccaagtgtagaagtccctccaccaccgccaaataggatgtaccctcctccacctccagcccttccctcctcagcaccttcagggcctccaccaccacctccatctgtgttgggggtagggccagtggcaccacccccaccgcctccacctccacctcctcctgggccaccgcccccgcctggcctgccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagacctcgagaccatggaaaagagaagatggaaaaagaatttcatagccgtctcagcagccaaccgctttaagaaaatctcatcctccggggcacttgattatgatattccaactactgctagcgagaatttgtattttcagggtgagctcaaaaccgcggctcttgcgcaacacgatgaagccgtggacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatggtgctcaggcgccgaaagtagacgcgaattccgcggggaagtcaaccggatccatctag

SEQ ID NO: 16 - protein, 716 aa, Artificial
  Myc-N-WASP-TAP fusion protein
MGEQKLISEEDLNDPSHWRPLEMSSVQQQPPPPRRVTNVGSLLLTPQENESLFTFLGKKCVTMSSAVVQLYAADRNCMWSKKCSGVACLVKDNPQRSHFLRIFDIKDGKLLWEQELYNNFVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKGDIGTPSNFQHIGHVGWDPNTGSDLNNLDPELKNLFDMCGILEAQLKERETLKVIYDFIEKTGGVEAVKNELRRQAPPPPPPSRGGPPPPPPPPHSSGPPPPPARGRGAPPPPPSRAPTAAPPPPPPSRPSVEVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWEDLETMEKRRWKKNFIAVSAANRFKKISSSGALDYDIPTTASENLYFQGELKTAALAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPKVDANSAGKSTGSI

SEQ ID NO: 17 - DNA, 1950 bp, Artificial
  Nucleotide sequence encoding GST-105WASP fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggctccgcggccgcccccttcaccgaaaacctgtattttcagggccttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactag

SEQ ID NO: 18 - protein, 649 aa, Artificial
  GST-105WASP fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGSAAAPFTENLYFQGLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 19 - DNA, 1827 bp, Artificial
  Nucleotide sequence encoding Myc-105WASP-TAP
atgggagagcagaaactgatctctgaagaagacctgaacgatccatcacactggcggccgctcgagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgacctcgagaccatggaaaagagaagatggaaaaagaatttcatagccgtctcagcagccaaccgctttaagaaaatctcatcctccggggcacttgattatgatattccaactactgctagcgagaatttgtattttcagggtgagctcaaaaccgcggctcttgcgcaacacgatgaagccgtggacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatggtgctcaggcgccgaaagtagacgcgaattccgcggggaagtcaaccggatccatc

SEQ ID NO: 20 - protein, 609 aa, Artificial
  Myc-105WASP-TAP
MGEQKLISEEDLNDPSHWRPLELVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDDLETMEKRRWKKNFIAVSAANRFKKISSSGALDYDIPTTASENLYFQGELKTAALAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPKVDANSAGKSTGSI

SEQ ID NO: 21 - DNA, 1962 bp, Artificial
  Nucleotide sequence encoding GST-tev-98N-WASP fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgaaaacctgtattttcagggctttgtatataatagtcctagaggatattttcatacctttgctggagatacttgtcaagttgctcttaattttgccaatgaagaagaagcaaaaaaatttcgaaaagcagttacagaccttttgggccgtcgacaaaggaaatctgagaaaagacgagatcccccaaatggtcctaatctacccatggctacagttgatataaaaaatccagaaatcacaacaaatagattttatggtccacaagtcaacaacatctcccataccaaagaaaagaagaagggaaaagctaaaaagaagagattaaccaaggcagatataggaacaccaagcaatttccagcacattggacatgttggttgggatccaaatacaggctttgatctgaataatttggatccagaattgaagaatcttttcgatatgtgtggaatctcagaggcacaacttaaagacagagaaacatcaaaagttatatatgactttattgaaaaaacaggaggtgttgaagctgttaaaaatgaactgcggaggcaagcaccaccacctccaccaccatcaaggggagggccacctcctcctcctccccctccacacaactcaggtcctcctcctcctcctgctaggggaagaggcgctcctcccccaccaccttcaagagctcccacagctgcacctccaccaccgcctccttccaggccaagtgtagcagtccctccaccaccgccaaataggatgtaccctcctccacctccagcccttccctcctcagcaccttcagggcctccaccaccacctccatctgtgttgggggtagggccagtggcaccacccccaccgcctccacctccacctcctcctgggccaccgcccccgcctggcctgccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagactag

SEQ ID NO: 22 - protein, 649 aa, Artificial; identical to SEQ ID NO: 18
  GST-tev-98N-WASP fusion protein

SEQ ID NO: 23 - DNA, 1914 bp, Artificial
  Nucleotide sequence encoding Myc-98N-WASP-TAP
atgggagagcagaaactgatctctgaagaagacctgaacgatatcacaagtttgtacaaaaaagcaggcttctttgtatataatagtcctagaggatattttcatacctttgctggagatacttgtcaagttgctcttaattttgccaatgaagaagaagcaaaaaaatttcgaaaagcagttacagaccttttgggccgtcgacaaaggaaatctgagaaaagacgagatcccccaaatggtcctaatctacccatggctacagttgatataaaaaatccagaaatcacaacaaatagattttatggtccacaagtcaacaacatctcccataccaaagaaaagaagaagggaaaagctaaaaagaagagattaaccaaggcagatataggaacaccaagcaatttccagcacattggacatgttggttgggatccaaatacaggctttgatctgaataatttggatccagaattgaagaatcttttcgatatgtgtggaatctcagaggcacaacttaaagacagagaaacatcaaaagttatatatgactttattgaaaaaacaggaggtgttgaagctgttaaaaatgaactgcggaggcaagcaccaccacctccaccaccatcaaggggagggccacctcctcctcctccccctccacacaactcaggtcctcctcctcctcctgctaggggaagaggcgctcctcccccaccaccttcaagagctcccacagctgcacctccaccaccgcctccttccaggccaagtgtagcagtccctccaccaccgccaaataggatgtaccctcctccacctccagcccttccctcctcagcaccttcagggcctccaccaccacctccatctgtgttgggggtagggccagtggcaccacccccaccgcctccacctccacctcctcctgggccaccgcccccgcctggcctgccttctgatggggaccatcaggttccaactactgcaggaaacaaagcagctcttttagatcaaattagagagggtgctcagctaaaaaaagtggagcagaacagtcggccagtgtcctgctctggacgagatgcactgttagaccagatacgacagggtatccaactaaaatctgtggctgatggccaagagtctacaccaccaacacctgcacccacttcaggaattgtgggtgcattaatggaagtgatgcagaaaaggagcaaagccattcattcttcagatgaagatgaagatgaagatgatgaagaagattttgaggatgatgatgagtgggaagacgacccagctttcttgtacaaagtggttgatatcccatcacactggcggccgctcgagaccatggaaaagagaagatggaaaaagaatttcatagccgtctcagcagccaaccgctttaagaaaatctcatcctccggggcacttgattatgatattccaactactgctagcgagaatttgtattttcagggtgagctcaaaaccgcggctcttgcgcaacacgatgaagccgtggacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatggtgctcaggcgccgaaagtagacgcgaattccgcggggaagtcaaccggatccatc

SEQ ID NO: 24 - protein, 638 aa, Artificial
  Myc-98N-WASP-TAP
MGEQKLISEEDLNDITSLYKKAGFFVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKADIGTPSNFQHIGHVGWDPNTGFDLNNLDPELKNLFDMCGISEAQLKDRETSKVIYDFIEKTGGVEAVKNELRRQAPPPPPPSRGGPPPPPPPPHNSGPPPPPARGRGAPPPPPSRAPTAAPPPPPPSRPSVAVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWEDDPAFLYKVVDIPSHWRPLETMEKRRWKKNFIAVSAANRFKKISSSGALDYDIPTTASENLYFQGELKTAALAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPKVDANSAGKSTGSI

SEQ ID NO: 25 - DNA, 1308 bp, Artificial
  Nucleotide sequence encoding GST-tev-Cdc42 fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgaaaacctgtattttcagggccagacaattaagtgtgttgttgtgggcgatgttgctgttggtaaaacatgtctcctgatatcctacacaacaaacaaatttccatcggaatatgtaccgactgtttttgacaactatgcagtcacagttatgattggtggagaaccatatactcttggactttttgatactgcagggcaagaggattatgacagattacgaccgctgagttatccacaaacagatgtatttctagtctgtttttcagtggtctctccatcttcatttgaaaacgtgaaagaaaagtgggtgcctgagataactcaccactgtccaaagactcctttcttgcttgttgggactcaaattgatctcagagatgacccctctactattgagaaacttgccaagaacaaacagaagcctatcactccagagactgctgaaaagctggcccgtgacctgaaggctgtcaagtatgtggagtgttctgcacttacacagagaggtctgaagaatgtgtttgatgaggctatcctagctgccctcgagcctccggaaactcaacccaaaaggaagtgctgtatattctag

SEQ ID NO: 26 - protein, 435 aa, Artificial
  GST-tev-Cdc42 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFENLYFQGQTIKCVVVGDVAVGKTCLLISYTTNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQRGLKNVFDEAILAALEPPETQPKRKCCIF

SEQ ID NO: 27 - DNA, 1314 bp, Artificial
  Nucleotide sequence encoding GST-tev-RhoC fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgaaaacctgtattttcagggcgctgcaatccgaaagaagctggtgatcgttggggatgttgcctgtgggaagacctgcctcctcatcgtcttcagcaaggatcagtttccggaggtctacgtccctactgtctttgagaactatattgcggacattgaggtggacggcaagcaggtggagctggctctgtgggacacagcagggcaggaagactatgatcgactgcggcctctctcctacccggacactgatgtcatcctcatgtgcttctccatcgacagccctgacagcctggaaaacattcctgagaagtggaccccagaggtgaagcacttctgccccaacgtgcccatcatcctggtggggaataagaaggacctgaggcaagacgagcacaccaggagagagctggccaagatgaagcaggagcccgttcggtctgaggaaggccgggacatggcgaaccggatcagtgcctttggctaccttgagtgctcagccaagaccaaggagggagtgcgggaggtgtttgagatggccactcgggctggcctccaggtccgcaagaacaagcgtcggaggggctgtcccattctctag

SEQ ID NO: 28 - protein, 437 aa, Artificial
  GST-tev-RhoC fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFENLYFQGAAIRKKLVIVGDVACGKTCLLIVFSKDQFPEVYVPTVFENYIADIEVDGKQVELALWDTAGQEDYDRLRPLSYPDTDVILMCFSIDSPDSLENIPEKWTPEVKHFCPNVPIILVGNKKDLRQDEHTRRELAKMKQEPVRSEEGRDMANRISAFGYLECSAKTKEGVREVFEMATRAGLQVRKNKRRRGCPIL

SEQ ID NO: 29 - DNA, 1314 bp, Artificial
  Nucleotide sequence encoding GST-tev-RhoA fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgaaaacctgtattttcagggcgctgccatccggaagaaactggtgattgttggtgatgtagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctggttgccttgtcttgtga

SEQ ID NO: 30 - protein, 437 aa, Artificial
  GST-tev-RhoA fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFENLYFQGAAIRKKLVIVGDVACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 31 - DNA, 1311 bp, Artificial
  Nucleotide sequence encoding GST-tev-Rac1 fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgaaaacctgtattttcagggccaggccatcaagtgtgtggtggtgggagacgtagctgtaggtaaaacttgcctactgatcagttacacaaccaatgcatttcctggagaatatatccctactgtctttgacaattattctgccaatgttatggtagatggaaaaccggtgaatctgggcttatgggatacagctggacaagaagattatgacagattacgccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgaagaagaggaagagaaaatgcctgctgttgtaa

SEQ ID NO: 32 - protein, 436 aa, Artificial
  GST-tev-Rac1 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFENLYFQGQAIKCVVVGDVAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 33 - DNA, 1845 bp, Artificial
  Nucleotide sequence encoding GST-tev-Nck1 fusion protein
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcgcagaagaagtggtggtagtagccaaatttgattatgtggcccaacaagaacaagagttggacatcaagaagaatgagagattatggcttctggatgattctaagtcctggtggcgagttcgaaattccatgaataaaacaggttttgtgccttctaactatgtggaaaggaaaaacagtgctcggaaagcatctattgtgaaaaacctaaaggataccttaggcattggaaaagtgaaaagaaaacctagtgtgccagattctgcatctcctgctgatgatagttttgttgacccaggggaacgtctctatgacctcaacatgcccgcttatgtgaaatttaactacatggctgagagagaggatgaattatcattgataaaggggacaaaggtgatcgtcatggagaaatgcagtgatgggtggtggcgtggtagctacaatggacaagttggatggttcccttcaaactatgtaactgaagaaggtgacagtcctttgggtgaccatgtgggttctctgtcagagaaattagcagcagtcgtcaataacctaaatactgggcaagtgttgcatgtggtacaggctctttacccattcagctcatctaatgatgaagaacttaatttcgagaaaggagatgtaatggatgttattgaaaaacctgaaaatgacccagagtggtggaaatgcaggaagatcaatggtatggttggtctagtaccaaaaaactatgttaccgttatgcagaataatccattaacttcaggtttggaaccatcacctccacagtgtgattacattaggccttcactcactggaaagtttgctggcaatccttggtattatggcaaagtcaccaggcatcaagcagaaatggcattaaatgaaagaggacatgaaggggatttcctcattcgtgatagtgaatcttcgccaaatgatttctcagtatcactaaaagcacaagggaaaaacaagcattttaaagtccaactaaaagagactgtctactgcattgggcagcgtaaattcagcaccatggaagaacttgtagaacattacaaaaaggcaccaatttttacaagtgaacaaggagaaaaattatatcttgtcaagcatttatcatga

SEQ ID NO: 34 - protein, 614 aa, Artificial
  GST-tev-Nck1 fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFAEEVVVVAKFDYVAQQEQELDIKKNERLWLLDDSKSWWRVRNSMNKTGFVPSNYVERKNSARKASIVKNLKDTLGIGKVKRKPSVPDSASPADDSFVDPGERLYDLNMPAYVKFNYMAEREDELSLIKGTKVIVMEKCSDGWWRGSYNGQVGWFPSNYVTEEGDSPLGDHVGSLSEKLAAVVNNLNTGQVLHVVQALYPFSSSNDEELNFEKGDVMDVIEKPENDPEWWKCRKINGMVGLVPKNYVTVMQNNPLTSGLEPSPPQCDYIRPSLTGKFAGNPWYYGKVTRHQAEMALNERGHEGDFLIRDSESSPNDFSVSLKAQGKNKHFKVQLKETVYCIGQRKFSTMEELVEHYKKAPIFTSEQGEKLYLVKHLS

SEQ ID NO: 35 - DNA, 561 bp, Artificial
  TAP tag (i.e., CBP, tev cleavage site and Prot A) nucleotide
atggaaaagagaagatggaaaaagaatttcatagccgtctcagcagccaaccgctttaagaaaatctcatcctccggggcacttgattatgatattccaactactgctagcgagaatttgtattttcagggtgagctcaaaaccgcggctcttgcgcaacacgatgaagccgtggacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatgatgctcaggcgccgaaagtagacaacaaattcaacaaagaacaacaaaacgcgttctatgagatcttacatttacctaacttaaacgaagaacaacgaaacgccttcatccaaagtttaaaagatgacccaagccaaagcgctaaccttttagcagaagctaaaaagctaaatggtgctcaggcgccgaaagtagacgcgaattccgcggggaagtcaaccggatccatctag

SEQ ID NO: 36 - protein, 186 aa, Artificial
  TAP tag (i.e., CBP, tev cleavage site and Prot A)
MEKRRWKKNFIAVSAANRFKKISSSGALDYDIPTTASENLYFQGELKTAALAQHDEAVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPKVDANSAGKSTGSI

SEQ ID NO: 37 - DNA, 1758 bp, Homo sapiens
  misc_feature (1)..(1758): SCAR1/WAVE1
atgccgctagtgaaaagaaacatcgatcctaggcacttgtgccacacagcactgcctagaggcattaagaatgaactggaatgtgtaaccaatatttccttggcaaatataattagacaactaagtagcctaagtaaatatgctgaagatatatttggagaattattcaatgaagcacatagtttttccttcagagtcaactcattgcaagaacgtgtggaccgtttatctgttagtgttacacagcttgatccaaaggaagaagaattgtctttgcaagatataacaatgaggaaagctttccgaagttctacaattcaagaccagcagcttttcgatcgcaagactttgcctattccattacaggagacgtacgatgtttgtgaacagcctccacctctcaatatactcactccttatagagatgatggtaaagaaggtctgaagttttataccaatccttcgtatttctttgatctatggaaagaaaaaatgttgcaagatacagaggataagaggaaggaaaagaggaagcagaagcagaaaaatctagatcgtcctcatgaaccagaaaaagtgccaagagcacctcatgacaggcggcgagaatggcagaagctggcccaaggtccagagctggctgaagatgatgctaatctcttacataagcatattgaagttgctaatggcccagcctctcattttgaaacaagacctcagacatacgtggatcatatggatggatcttactcactttctgccttgccatttagtcagatgagtgagcttctgactagagctgaggaaagggtattagtcagaccacatgaaccacctccacctccaccaatgcatggagcaggagatgcaaaaccgatacccacctgtatcagttctgctacaggtttgatagaaaatcgccctcagtcaccagctacaggcagaacacctgtgtttgtgagccccactcccccacctcctccaccacctcttccatctgccttgtcaacttcctcattaagagcttcaatgacttcaactcctccccctccagtacctcccccacctccacctccagccactgctttgcaagctccagcagtaccaccacctccagctcctcttcagattgcccctggagttcttcacccagctcctcctccaattgcacctcctctagtacagccctctccaccagtagctagagctgccccagtatgtgagactgtaccagttcatccactcccacaaggtgaagttcaggggctgcctccacccccaccaccgcctcctctgcctccacctggcattcgaccatcatcacctgtcacagttacagctcttgctcatcctccctctgggctacatccaactccatctactgccccaggtccccatgttccattaatgcctccatctcctccatcacaagttatacctgcttctgagccaaagcgccatccatcaaccctacctgtaatcagtgatgccaggagtgtgctactggaagcaatacgaaaaggtattcagctacgcaaagtagaagagcagcgtgaacaggaagctaagcatgaacgcattgaaaacgatgttgccaccatcctgtctcgccgtattgctgttgaatatagtgattcggaagatgattcagaatttgatgaagtagattggttggagtaagaaaaatgcattgataaatattacaaaactgaatgcaaatgtcctttgtggtgcttgttccttgaaaatgtttggtca

SEQ ID NO: 38 - protein, 559 aa, Homo sapiens
  misc_feature (1)..(559): SCAR1/WAVE1
MPLVKRNIDPRHLCHTALPRGIKNELECVTNISLANIIRQLSSLSKYAEDIFGELFNEAHSFSFRVNSLQERVDRLSVSVTQLDPKEEELSLQDITMRKAFRSSTIQDQQLFDRKTLPIPLQETYDVCEQPPPLNILTPYRDDGKEGLKFYTNPSYFFDLWKEKMLQDTEDKRKEKRKQKQKNLDRPHEPEKVPRAPHDRRREWQKLAQGPELAEDDANLLHKHIEVANGPASHFETRPQTYVDHMDGSYSLSALPFSQMSELLTRAEERVLVRPHEPPPPPPMHGAGDAKPIPTCISSATGLIENRPQSPATGRTPVFVSPTPPPPPPPLPSALSTSSLRASMTSTPPPPVPPPPPPPATALQAPAVPPPPAPLQIAPGVLHPAPPPIAPPLVQPSPPVARAAPVCETVPVHPLPQGEVQGLPPPPPPPPLPPPGIRPSSPVTVTALAHPPSGLHPTPSTAPGPHVPLMPPSPPSQVIPASEPKRHPSTLPVISDARSVLLEAIRKGIQLRKVEEQREQEAKHERIENDVATILSRRIAVEYSDSEDDSEFDEVDWLE

SEQ ID NO: 39 - DNA, 4270 bp, Homo sapiens
  misc_feature (1)..(4270): SCAR2/WAVE2
ggggaatcgcgtaatggcggacacaggcagggcgagcgcggctgggggcgtagcgcgctgagggggtccggccgtttggcagcccgcgaggcggtccgcgggagcacactctgtgcggagactgggcggccggccgacccttcctgtcgctgacggcgactgcgggaggccaggttgtttttcaccattcagaacattgcctgaagcaggtccaccatgccgttagtaacgaggaacatcgagccaaggcacctgtgccgtcagacgttgcctagcgttagaagcgagctggaatgcgtgaccaacatcaccctggcaaatgtcatccgacagctgggcagcctgagtaaatatgcagaggacatttttggagagctctttactcaggcaaatacctttgcctctcgggtaagctcccttgctgagagggtcgaccgactacaggttaaagtcactcagctggatcccaaggaagaagaagtgtcactgcaaggaatcaacacccgaaaagccttcagaagttccaccattcaagaccagaagctttttgacagaaactctctcccagtgcctgtcttagaaacatacaatacctgtgatactcctccccctctcaacaatcttaccccttacagggacgatggaaaagaggcactcaaattctacacagacccttcatacttctttgatctttggaaggagaagatgctgcaggacaccaaggatatcatgaaagagaagagaaagcataggaaagaaaagaaagataatccaaatcgagggaatgtaaacccacgtaaaatcaagacacgtaaggaagagtgggagaaaatgaagatggggcaagaatttgtggagtccaaagaaaagctggggacttctgggtatccacccactttggtgtaccagaatggcagcattggctgtgttgaaaacgtggatgcaagtagctatccgccaccaccacagtcagactctgcttcttcaccttctccttccttctccgaggacaacttgcctcctccaccagcagaattcagttacccagtggacaaccaaagaggatctggtttggctggacccaaaagatccagtgtggtcagcccaagccatccaccaccagctcctcctctaggctctccaccaggccctaaacccgggtttgctccaccacctgcccctccgccacctccgcctccaatgataggcatcccacctccaccaccgcctgtaggatttgggtctccagggacgcctccaccaccctcacccccatctttcccacctcaccctgattttgctgcccctccacctcctcctccaccaccagcagctgactacccaactctgccaccacctcccttgtcccagccaacaggaggagcacctcctcctccccctcctcctcctcctccggggccccctcctccccctttcactggtgcagatggccagcctgctataccaccaccgctttctgataccaccaagcccaagtcctccttgcctgccgtgagcgatgcccgtagcgacctgctttcagccatccgtcaaggttttcagctgcgcagggttgaggagcagcgggaacaagagaagcgggatgttgtgggcaatgacgtggccaccatcttgtctcgtcgcattgctgttgagtacagtgactcagaagatgactcctctgaatttgatgaggacgactggtccgattaactctttctgcctgctgcccaccttctttttctttccttcctacctgccttctttgatgccaaccccaacagacccgtagggggagaaaagggaggaaaaaagtaattttaaggggccaaagctttccctgaagcaaccaaagatatatccaagtgcttcctccaagtcaacatgtatttcctctccccattttcaggccctgtggggctcctgaggttcagtagctgggatgttccctctttccttcaagtgcctgttgcatattgaaaggaaggagaaatcccaaagcagattcctttgatcgggtttctgttggagatggggcttcccttaggagccatattcaactacagccttctaaaacctgtgccctcagccacttcgaatgccagccaccttctggttctaaaacggggagtggtctgaatgaacacagctgacccctttcccgcgcactgaaagggcagagtaggccgaagggtccaagggccagactgcctcaccctctgccctaatcagcagggtgggcctgccttttgctaagcgatctctatgcctgggatgccctttattccaggaggcatcaagcctctaaagaatgtctcacctcctctgcccaaaaatgatgcctttctgtaggctggtgttgttgcctccctcccaggatccctttggtgagtatggtgttcaggatgcaccaccaccacctctagataccttcaggcaacacagcccagttttaacctctagtatccatgaccaaactatccctgacacatgaggacaggggcctcttctggctgtcaggagcaaagcctgaagacttggagctgcaggactggaagaacagtggagccccgtgggtctcaccctttaaggatgctgaggcctagagatgggaagtgacttgctcaaggtcacacaattggatagtgacatagctagagcgcagagttcctgattccaagtcacctgtgctttctgggaccaaagaatgggcacctgctggagtccgggcagagctttctcagttgtattgctactccagacctcaccataggttggggtcccagtaggaaggctcagggtctgtgccagccctgtcggtgctgctcagaccttcatagcctctcttgtcattctttgttgccccttttctgtcaccagccaaccacatagccttgggaccagcctctctgggggaccagaagtagtgagagaaggaaggggataggcagctttgacaggtgctgctttcaattcctctgcaactcctcccccttttatttccccaatttaaacaaagattctgccaactgtggaaacttcagtccctcaggctggcagccatgccagtacctgcctgggggtggggggtgcctggcagccatgaagcaggctgaaaggcagaggggctccaggtcctgtttccagctcccctcactgcacatggtgaagctcgctccctccctccctcccttcccgcttttcccagagctaatacacaggtgctattattcagaaaaaaactggtcagctctagccaacagtgaggtttcttttcttctgccctaactattgtgtagcctcttatgctgaaatcggcttctgctggcttctccggctttcagagccctgaaacaaagagaaacaggatctgtccctacccagcacagcaaatggttgtagtaattgccaaagccctcataaagccctccggcttgaggagagagtgtatagtcatgggttctgcctctgtgcccttgctggccgcttctcctctgccttctttcctggaactcagggtgtggggactgagcctgtaggggacagcatgccgtcttgctgtggccactcccaagtgtgccctcttccctctttacacatcaggtgtctctggcacaggacttggcactaagctccatgctgagacaccaggctatgtgggcccccaccttgtttcccagcctgcaccttagaagccgaaggtgctttcatcagaaccctaaaatggtcgttgaaggcgcctgggccgcagcccagcagtagttggagaggcaggcagagggcagtggttctcccaaataggagacctggggcctggccaggccagggtttgggcctaatggctttgactaaattacccccatcctccttgcccggaaaagggagagctagagccactcactgtcattctgctctgaccttgaagggggcggtgttggcctggcttctggaatggactgagtccatcgtggaaagggctgggggcaggaggaggtggggaggggcactgcctgcggaaggtaggattagatcattagctcagtgacctcctagggtttcgatgtgctatgttctcatcctacagttggtttggtaatgatctgcaagtcccggagagcaacagcacagctctgcctgacgctctcattaaaatctatgcagccaagctcggcactttgtagcagccggccttgcgaagcctcctcagctcggggggccggggacccagtgagccgagaggccctctgggctccacttatgcatatgcacc

SEQ ID NO: 40 - protein, 498 aa, Homo sapiens
  misc_feature (1)..(498): SCAR2/WAVE2
MPLVTRNIEPRHLCRQTLPSVRSELECVTNITLANVIRQLGSLSKYAEDIFGELFTQANTFASRVSSLAERVDRLQVKVTQLDPKEEEVSLQGINTRKAFRSSTIQDQKLFDRNSLPVPVLETYNTCDTPPPLNNLTPYRDDGKEALKFYTDPSYFFDLWKEKMLQDTKDIMKEKRKHRKEKKDNPNRGNVNPRKIKTRKEEWEKMKMGQEFVESKEKLGTSGYPPTLVYQNGSIGCVENVDASSYPPPPQSDSASSPSPSFSEDNLPPPPAEFSYPVDNQRGSGLAGPKRSSVVSPSHPPPAPPLGSPPGPKPGFAPPPAPPPPPPPMIGIPPPPPPVGFGSPGTPPPPSPPSFPPHPDFAAPPPPPPPPAADYPTLPPPPLSQPTGGAPPPPPPPPPPGPPPPPFTGADGQPAIPPPLSDTTKPKSSLPAVSDARSDLLSAIRQGFQLRRVEEQREQEKRDVVGNDVATILSRRIAVEYSDSEDDSSEFDEDDWSD

SEQ ID NO: 41 - DNA, 1509 bp, Homo sapiens
  misc_feature (1)..(1509): SCAR3/WAVE3
atgcctttagtgaagaggaacattgagccccggcacttgtgccggggagctctgcctgaagggattaccagcgaacttgaatgtgtaaccaatagtactcttgccgctatcatacgccagctgagcagtctgagcaaacatgctgaagacatatttggtgagttgtttaatgaggctaacaacttctacatcagagcaaattctcttcaagacagaattgatcgccttgctgtcaaagtcacccagctggattcaacagtggaagaggtctcactacaggatatcaacatgaaaaaagctttcaaaagttccacagtccaagaccagcaagtggtttcaaagaacagcattcctaatcctgttgctgatatttacaaccagagtgataagccaccgcctctgaacatcctgacaccatacagagatgacaagaaggatgggctgaagttctatactgatccttcctatttctttgacctctggaaagaaaaaatgctacaggacacagaagacaaaaggaaagagaaaaggcgtcaaaaggagcaaaagcgtatagatggcaccacccgtgaggtgaaaaaggttagaaaagccagaaacaggcgccaggagtggaatatgatggcatatgacaaagagcttagacccgacaacaggttgtctcagagtgtgtaccatggagcgtcttccgagggatccctgtccccagatactaggtcacatgcatcggacgttacggattactcttacccggctactcccaaccattctctgcacccccagcctgtgaccccttcctatgcagctggtgacgtgccaccacacgggcctgcaagccaggctgcggagcatgagtaccggcccccatctgcctcggcgaggcacatggccctcaacagacctcagcagccgcccccccggcgtccccctcaggccccagaggggtcccaggcctctgcaccgatggctccagcagactacgggatgctcccagcgcagataattgagtattacaacccatccggaccacctcctccgccacctcctcctgtgattccctcagcacaaactgccttcgtcagccctctccagatgcccatgcagcccccgttccctgcatcagccagctccacgcacgcagctcctcctcacccaccctccaccgggctcctggtcacagccccgccacccccgggcccaccacctcccccgccaggccctcctggtcccgggtcttctctttcgtcctccccaatgcatggccccccagtagctgaggcgaagcggcaagagcctgcacagccaccaatcagtgatgctcgaagcgacctcctcgctgctattcgaatgggaattcaactgaaaaaggtgcaggagcagcgggagcaggaggccaagcgggagccagtggggaatgacgtggccacgatcctgtcccggcgcattgccgtggagtacagcgactctgacgacgactcagagttcgacgagaacgactggtccgactga

SEQ ID NO: 42 - protein, 502 aa, Homo sapiens [sequence omitted]

SEQ ID NO: 43 - DNA, 1143 bp, Homo sapiens
  misc_feature (1)..(1143): NCK2
atgacagaagaagttattgtgatagccaagtgggactacaccgcccagcaggaccaggagctggacatcaagaagaacgagcggctgtggttgctggacgactccaagacgtggtggcgggtgaggaacgcggccaacaggacgggctatgtaccgtccaactacgtggagcggaagaacagcctgaagaagggctccctcgtgaagaacctgaaggacacactaggcctcggcaagacgcgcaggaagaccagcgcgcgggatgcgtcccccacgcccagcacggacgccgagtaccccgccaatggcagcggcgccgaccgcatctacgacctcaacatcccggccttcgtcaagttcgcctatgtggccgagcgggaggatgagttgtccctggtgaaggggtcgcgcgtcaccgtcatggagaagtgcagcgacggttggtggcggggcagctacaacgggcagatcggctggttcccctccaactacgtcttggaggaggtggacgaggcggctgcggagtccccaagcttcctgagcctgcgcaagggcgcctcgctgagcaatggccagggctcccgcgtgctgcatgtggtccagacgctgtaccccttcagctcagtcaccgaggaggagctcaacttcgagaagggggagaccatggaggtgattgagaagccggagaacgaccccgagtggtggaaatgcaaaaatgcccggggccaggtgggcctcgtccccaaaaactacgtggtggtcctcagtgacgggcctgccctgcaccctgcgcacgccccacagataagctacaccgggccctcgtccagcgggcgcttcgcgggcagagagtggtactacgggaacgtgacgcggcaccaggccgagtgcgccctcaacgagcggggcgtggagggcgacttcctcattagggacagcgagtcctcgcccagcgacttctccgtgtcccttaaagcgtcagggaagaacaaacacttcaaggtgcagctcgtggacaatgtctactgcattgggcagcggcgcttccacaccatggacgagctggtggaacactacaaaaaggcgcccatcttcaccagcgagcacggggagaagctctacctcgtcagggccctgcagtga

SEQ ID NO: 44 - protein, 380 aa, Homo sapiens
  misc_feature (1)..(380): NCK2
MTEEVIVIAKWDYTAQQDQELDIKKNERLWLLDDSKTWWRVRNAANRTGYVPSNYVERKNSLKKGSLVKNLKDTLGLGKTRRKTSARDASPTPSTDAEYPANGSGADRIYDLNIPAFVKFAYVAEREDELSLVKGSRVTVMEKCSDGWWRGSYNGQIGWFPSNYVLEEVDEAAAESPSFLSLRKGASLSNGQGSRVLHVVQTLYPFSSVTEEELNFEKGETMEVIEKPENDPEWWKCKNARGQVGLVPKNYVVVLSDGPALHPAHAPQISYTGPSSSGRFAGREWYYGNVTRHQAECALNERGVEGDFLIRDSESSPSDFSVSLKASGKNKHFKVQLVDNVYCIGQRRFHTMDELVEHYKKAPIFTSEHGEKLYLVRALQ

SEQ ID NO: 45 - DNA, 1854 bp, Homo sapiens
  misc_feature (1)..(1854): GST-NCK2
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtccatggtcgaatcaaacaagtttgtacaaaaaagcaggcttcacagaagaagttattgtgatagccaagtgggactacaccgcccagcaggaccaggagctggacatcaagaagaacgagcggctgtggttgctggacgactccaagacgtggtggcgggtgaggaacgcggccaacaggacgggctatgtaccgtccaactacgtggagcggaagaacagcctgaagaagggctccctcgtgaagaacctgaaggacacactaggcctcggcaagacgcgcaggaagaccagcgcgcgggatgcgtcccccacgcccagcacggacgccgagtaccccgccaatggcagcggcgccgaccgcatctacgacctcaacatcccggccttcgtcaagttcgcctatgtggccgagcgggaggatgagttgtccctggtgaaggggtcgcgcgtcaccgtcatggagaagtgcagcgacggttggtggcggggcagctacaacgggcagatcggctggttcccctccaactacgtcttggaggaggtggacgaggcggctgcggagtccccaagcttcctgagcctgcgcaagggcgcctcgctgagcaatggccagggctcccgcgtgctgcatgtggtccagacgctgtaccccttcagctcagtcaccgaggaggagctcaacttcgagaagggggagaccatggaggtgattgagaagccggagaacgaccccgagtggtggaaatgcaaaaatgcccggggccaggtgggcctcgtccccaaaaactacgtggtggtcctcagtgacgggcctgccctgcaccctgcgcacgccccacagataagctacaccgggccctcgtccagcgggcgcttcgcgggcagagagtggtactacgggaacgtgacgcggcaccaggccgagtgcgccctcaacgagcggggcgtggagggcgacttcctcattagggacagcgagtcctcgcccagcgacttctccgtgtcccttaaagcgtcagggaagaacaaacacttcaaggtgcagctcgtggacaatgtctactgcattgggcagcggcgcttccacaccatggacgagctggtggaacactacaaaaaggcgcccatcttcaccagcgagcacggggagaagctctacctcgtcagggccctgcagtga

SEQ ID NO: 46 - protein, 617 aa, Homo sapiens
  misc_feature (1)..(617): GST-NCK2
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFTEEVIVIAKWDYTAQQDQELDIKKNERLWLLDDSKTWWRVRNAANRTGYVPSNYVERKNSLKKGSLVKNLKDTLGLGKTRRKTSARDASPTPSTDAEYPANGSGADRIYDLNIPAFVKFAYVAEREDELSLVKGSRVTVMEKCSDGWWRGSYNGQIGWFPSNYVLEEVDEAAAESPSFLSLRKGASLSNGQGSRVLHVVQTLYPFSSVTEEELNFEKGETMEVIEKPENDPEWWKCKNARGQVGLVPKNYVVVLSDGPALHPAHAPQISYTGPSSSGRFAGREWYYGNVTRHQAECALNERGVEGDFLIRDSESSPSDFSVSLKASGKNKHFKVQLVDNVYCIGQRRFHTMDELVEHYKKAPIFTSEHGEKLYLVRALQ

SEQ ID NO: 47 - protein, 627 aa, Candida albicans
  misc_feature (1)..(627): FOR1 FH1-FH2 domain
MDPKFLQELSLKVGKAEPIQDANNKNQFGGPLSSSPEDVSQKHKTSGDSSDKDKVLSSPISSNDIKSPETGNSTTSSAAPPPPPPPPPPPPPPLPPILGGNNSSAAPPPPPPPPPPPAFLNGSGSVIPPAPPLPPPSSGRSSRSVPSTVTKSSGSAFDKIPRPKKKLKQLHWEKIDHSQVGNSFWNDPNTHTLVDDLMSKGIFDEIELIFAAKEAKKLATKKKEDLDKVTFLARDISQQFSINLHAFNSFSDEEFVLKVLRCDKDVLTNPAVLDFFGKEDIVEITNTLARNFEPYSTDYKTEEITKPEKDPNELQRPDRIYLELMYNLQHYWKSRTRALNVVVNYDKDVYEYVKKLRLIDEAVDSIKNSKHLKGVFEIILAVGNYMNDSAKQAHGFKLSSLQRLSFMKDEKNSMTFLHYVEKVIRTQYPEFLEFINELSCCNEITKFSIENINNDCKEYARAIKNVQSSIDIGNLSDVSKFHPSDRVLKAVLPALPRAKRKAELLLDQANYTMKEFDDLMKYFGEDPTDQFVKNSFISKFTDFMKDFKRVQAENIKREEELRVYEQRKKLLEKPKSSNNGDSNASDQDGESNEGDGGVMDSLLQRLKAAAPTKGESASARKKALMRK

SEQ ID NO: 48 - protein, 472 aa, Candida albicans
  misc_feature (1)..(472): FOR1 FH2 domain
AFDKIPRPKKKLKQLHWEKIDHSQVGNSFWNDPNTHTLVDDLMSKGIFDEIELIFAAKEAKKLATKKKEDLDKVTFLARDISQQFSINLHAFNSFSDEEFVLKVLRCDKDVLTNPAVLDFFGKEDIVEITNTLARNFEPYSTDYKTEEITKPEKDPNELQRPDRIYLELMYNLQHYWKSRTRALNVVVNYDKDVYEYVKKLRLIDEAVDSIKNSKHLKGVFEIILAVGNYMNDSAKQAHGFKLSSLQRLSFMKDEKNSMTFLHYVEKVIRTQYPEFLEFINELSCCNEITKFSIENINNDCKEYARAIKNVQSSIDIGNLSDVSKFHPSDRVLKAVLPALPRAKRKAELLLDQANYTMKEFDDLMKYFGEDPTDQFVKNSFISKFTDFMKDFKRVQAENIKREEELRVYEQRKKLLEKPKSSNNGDSNASDQDGESNEGDGGVMDSLLQRLKAAAPTKGESASARKKALMRK

SEQ ID NO: 49 - DNA, 77 bp, Artificial
  WASP forward primer
ggggacaagtttgtacaaaaaagcaggcttcgaaaacctgtattttcagggcgggggtcggggagcgcttttggatc

SEQ ID NO: 50 - DNA, 84 bp, Artificial
  WASP reverse primer
ggggaccactttgtacaagaaagctgggtcctagtgatggtgatggtgatggtagtacgagtcatcccattcatcatcttcatc

SEQ ID NO: 51 - DNA, 57 bp, Artificial
  WASP VCA domain reverse primer
ggggaccactttgtacaagaaagctgggtcctagtcatcccattcatcatcttcatc

SEQ ID NO: 52 - DNA, 49 bp, Artificial
  WASP forward primer
caccgaaaacctgtattttcagggccttgtctactccacccccaccccc

SEQ ID NO: 53 - DNA, 24 bp, Artificial
  WASP reverse primer
ctagtcatcccattcatcatcttc

SEQ ID NO: 54 - DNA, 55 bp, Artificial
  WASP forward primer
ggggacaagtttgtacaaaaaagcaggcttccttgtctactccacccccaccccc

SEQ ID NO: 55 - DNA, 54 bp, Artificial
  WASP reverse primer
ggggaccactttgtacaagaaagctgggtcgtcatcccattcatcatcttcatc

SEQ ID NO: 56 - DNA, 54 bp, Artificial
  WASP forward primer
ggggacaagtttgtacaaaaaagcaggcttcatgagtgggggcccaatgggagg

SEQ ID NO: 57 - DNA, 70 bp, Artificial
  N-WASP forward primer
ggggacaagtttgtacaaaaaagcaggcttcgaaaacctgtattttcagggctctgatggggaccatcag

SEQ ID NO: 58 - DNA, 57 bp, Artificial
  N-WASP reverse primer
ggggaccactttgtacaagaaagctgggtcctagtcttcccactcatcatcatcctc

SEQ ID NO: 59 - DNA, 56 bp, Artificial
  pENTR/SD/TOPO_NWASP forward primer
caccgaaaacctgtattttcagggctttgtatataatagtcctagaggatattttc

SEQ ID NO: 60 - DNA, 24 bp, Artificial
  pENTR/SD/TOPO_NWASP reverse primer
ttagtcttcccactcatcatcatc

SEQ ID NO: 61 - DNA, 75 bp, Artificial
  pENTR/SD/TOPO_tev_98FNWASP forward primer
ggggacaagtttgtacaaaaaagcaggcttcgaaaacctgtattttcagggctttgtatataatagtcctagagg

SEQ ID NO: 62 - DNA, 54 bp, Artificial
  pENTR/SD/TOPO_tev_98FNWASP reverse primer
ggggaccactttgtacaagaaagctgggtcgtcttcccactcatcatcatcctc

SEQ ID NO: 63 - DNA, 49 bp, Artificial
  N-WASP forward primer
caccgaaaacctgtattttcagggcagctccgtccagcagcagccgccg

SEQ ID NO: 64 - DNA, 24 bp, Artificial
  N-WASP reverse primer
tcagtcttcccactcatcatcatc

SEQ ID NO: 65 - DNA, 31 bp, Artificial
  pENTR_N-WASP/sd/TOPO forward primer
gccgctcgaggtcttcccactcatcatcatc

SEQ ID NO: 66 - DNA, 29 bp, Artificial
  pENTR_N-WASP/sd/TOPO reverse primer
gccgctcgagatgagctccgtccagcagc

SEQ ID NO: 67 - DNA, 39 bp, Artificial
  pDONR_tev_Cdc42 forward primer
tgtgttgttgtgggcgatgttgctgttggtaaaacatgt

SEQ ID NO: 68 - DNA, 39 bp, Artificial
  pDONR_tev_Cdc42 reverse primer
acatgttttaccaacagcaacatcgcccacaacaacaca

SEQ ID NO: 69 - DNA, 33 bp, Artificial
  pDONR_tev_RhoC forward primer
gtgatcgttggggatgttgcctgtgggaaggac

SEQ ID NO: 70 - DNA, 33 bp, Artificial
  pDONR_tev_RhoC reverse primer
gtccttcccacaggcaacatccccaacgatcac

SEQ ID NO: 71 - DNA, 76 bp, Artificial
  RhoA GTP forward primer
ggggacaagtttgtacaaaaaagcaggcttcgaaaacctgtattttcagggcgctgccatccggaagaaactggtg

SEQ ID NO: 72 - DNA, 58 bp, Artificial
  RhoA GTP reverse primer
ggggaccactttgtacaagaaagctgggtcctacaagacaaggcaaccacattttttc

SEQ ID NO: 73 - DNA, 76 bp, Artificial
  Rac1 forward primer
ggggacaagtttgtacaaaaaaacgggcttcgaaaacctgtattttcagggccaggccatcaagtgtgtggtggtg

SEQ ID NO: 74 - DNA, 58 bp, Artificial
  Rac1 reverse primer
ggggaccactttgtacaagaaagctgggtcctacaacagcaggcattttctcttcctc

SEQ ID NO: 75 - DNA, 77 bp, Artificial
  Nck forward primer
ggggacaagtttgtacaaaaaagcaggcttcgaaaacctgtattttcagggcatggcagaagaagtggtggtagtag

SEQ ID NO: 76 - DNA, 57 bp, Artificial
  Nck reverse primer
ggggaccactttgtacaagaaagctgggtcctatgataaatgcttgacaagatataa

SEQ ID NO: 77 - DNA, 31 bp, Artificial
  NCK2 forward primer
caccatgacagaagaagttattgtgatagcc

SEQ ID NO: 78 - DNA, 27 bp, Artificial
  NCK2 reverse primer
tcactgcagggccctgacgaggtagag

(19) United States
(12) Patent Application Publication
(10) Pub. No.: US 2006/0003399 A1
(43) Pub. Date: Jan. 5, 2006

(54) High throughput actin polymerization assay

(75) Inventors: Nenad Tomasevic, Foster City, CA (US); Zhiheng Jia, Fremont, CA (US); Roman Sakowicz, Foster City, CA (US); Daniel Pierce, Hayward, CA (US); Jeffrey Finer, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cytokinetics, Inc., South San Francisco, CA (US)

(21) Appl. No.: 11/150,845

(22) Filed: Jun. 10, 2005

Related U.S. Application Data
(60) Provisional application No. 60/673,444, filed on Apr. 20, 2005.
(60) Provisional application No. 60/578,949, filed on Jun. 10, 2004.

(51) Int. Cl. C12Q 1/37

(52) U.S. Cl. 435/23

(57) ABSTRACT

Methods for assaying for actin polymerization are described, as are the use of the assays to screen for modulators of actin polymerization. The screening methods can be utilized to identify active agents that interact with actin, the polymerization state of actin and other proteins or cellular components whose function is naturally or artificially coupled to actin polymerization or depolymerization. Protein constructs and kits useful in performing the methods are also provided.

### CROSS-REFERENCES TO RELATED APPLICATIONS

This application claims the benefit of U.S. Provisional Application Nos. 60/578,949, filed Jun. 10, 2004, and 60/673,444, filed Apr. 20, 2004, both of which are incorporated herein by reference in their entirety for all purposes. This application is related to U.S. Application No. ______, filed ______, which claims the benefit of U.S. Provisional Application No. 60/578,969, filed Jun. 10, 2004, both of which are incorporated herein by reference in their entirety for all purposes. This application is also related to U.S. Application No. ______, filed ______, which claims the benefit of U.S. Provisional Application No. 60/578,913, filed Jun. 10, 2004, both of which are incorporated herein by reference in their entirety for all purposes.

### BACKGROUND

Directed movement of cells plays an essential role in the pathogenesis of many human diseases. These include chronic inflammatory diseases such as rheumatoid arthritis (Jenkins, J. K. et al. (2002) Am J Med Sci 323:171-180; Szekanecz, Z. et al. (2000) Arthritis Res 2:368-373), inflammatory bowel disease (Salmi, M. et al. (1998) Inflamm. Bowel Dis. 4:149-156) and atherosclerosis (Kraemer, R. (2000) Curr. Atheroscler. Rep. 2:445-452), and the metastatic spread of solid tumors (Chambers, A. F. et al. (2000) Breast Cancer Res. 2:400-407; Condeelis, J. S. et al. (2001) Semin. Cancer Biol. 11:119-128). Cell types playing major roles in inflammation are largely hematopoietic in origin, deriving from lymphoid (T-cells), granulocytic (neutrophils, eosinophils) and monocytic (monocytes, macrophages, osteoclasts) lineages. These cells are attracted to the diseased site by chemotactic stimuli, and must move from the circulatory system through vessel endothelium to the diseased site.

Dissemination of tumor cells from a primary site to secondary sites is dependent upon the escape from the primary tumor, intravasation into the lymphatic or vascular system, extravasation and growth. Extravasation of both inflammatory cells and tumor cells involves adherence to the luminal endothelium of a blood vessel, followed by transendothelial migration and colonization of the secondary site. In both inflammatory diseases and metastasis, sites of extravasation are thought to be determined in part by chemoattractive signals derived from a target tissue to which the tumor or inflammatory cells are receptive (Muller, A. et al. (2001) Nature 410:50-56). Survival and sustained proliferation of tumor metastases is dependent upon the development of a tumor vascular system to deliver nutrients to the growing tumor (Zetter, B. R. (1998) Annu. Rev. Med. 49:407424). This process, called angiogenesis, involves the proliferation and migration of vascular and perhaps bone marrow-derived endothelial cells in response to chemoattractive factors secreted by the tumor, resulting in the growth of new blood vessels.

The actin cytoskeleton and the proteins that regulate its formation play a central role in cell movement and polarity, and thus are useful targets for the treatment of inflammatory diseases and for preventing metastatic spread of primary cancers. Polarized cell movement is driven by reorganization of the cortical actin cytoskeleton at the leading edge of moving cells, resulting in the production of a propulsive force (Higgs, H. N. et al. (2001) Annu. Rev. Biochem. 70:649-676; Small, J. V. et al. (2002) Trends Cell Biol. 12:112-120). The actin cytoskeleton also plays a role in changes in cell shape and in the internalization of extracellular materials via endocytosis and phagocytosis.

These processes depend upon the rapid and localized assembly and disassembly of actin filaments. New filaments are created by nucleation of monomeric actin (Carson, M. et al. (1986) J. Cell Biol. 103:2707-2714; Chan, A. Y. et al. (1998) J. Cell Sci. 111:199-211), which refers to the initiation of actin polymerization from free actin monomers (globular actin or G-actin) into filamentous actin (F-actin), and is the rate-limiting step in the assembly of actin filaments. The very large kinetic barrier to nucleation indicates that regulation of the nucleation step may be critical to controlling actin polymerization in cells.

The actin nucleation machinery includes at least two key components: the Arp2/3 complex and one or more members from the family of nucleation promoting factors (NPFs). The Arp2/3 complex (or simply Arp2/3) is responsible for nucleating new actin filaments and cross-linking newly formed filaments into Y-branched arrays. In particular, the Arp2/3 complex is positioned at the Y-branch between the filaments and stabilizes the cross-link region. The Arp2/3 complex consists of six subunits in Saccharomyces cerevisiae and seven subunits in Acanthaemoeba castellanii and humans. The two largest subunits (50 and 43 kDa) are actin-related proteins in the Arp3 (also sometimes referred to as ACTR2) and Arp2 (sometimes referred to as ACTR3) families, respectively. The name of the complex is thus named after these two subunits. The other five subunits in the human complex have molecular masses of approximately 41, 34, 21, 20 and 16 kDa, based upon sodium dodecyl sulfate-polyacrylamide gel electrophoresis (SDS-PAGE) studies and the subunits from humans are referred to as p41-Arc, p34-Arc, p21-Arc, p20-Arc and p16-Arc, respectively.

Arp2/3 by itself, however, possesses little activity. The complex must be bound by a NPF to become activated. Examples of such NPFs include Wiskott-Aldrich Syndrome protein (WASP), a WASP homolog called N-WASP, and a family of proteins called suppressor of cAR (SCAR) (also referred to as the WASP family verprolin homologous (WAVE) proteins). The SCAR/WAVE family includes SCAR1/WAVE1, SCAR2/WAVE2 and SCAR3/WAVE3. See, for example, Welch, M. D. and Mullins, R. D. (2002) Annu. Rev. Cell Dev. Biol. 18:247-288; Higgs, H. N. and Pollard, T. D. (2001) Annu. Rev. Biochem. 70:649-76; Caron, E. (2002) Curr. Opin. Cell Biol. 14:82-87; and Takenawa, T. (2001) J. Cell Sci. 114:1801-1809, each of which are incorporated herein by reference in their entirety for all purposes. WASP is expressed exclusively in hematopoietic cells, N-WASP and WAVE2 are ubiquitously expressed and WAVE1 and WAVE3 are expressed exclusively in the neurons.

Once a nucleation promoting factor (NPF) has bound Arp2/3 to form an activated conformation, the complex serves as a nucleus for polymerization of G-ATP-actin and mimics the barbed end of an actin filament. During the nucleation process, the Arp2/3 complex binds to the side of an existing actin filament. Filament binding in the absence of an activator, or activator interaction in the absence of a pre-existing actin filament, does not result in appreciable Arp2/3 activity. Arp2/3 does not interact with the ends of filaments in any manner except with the filament that it itself has nucleated.

NPFs are also regulated. They are activated by the binding of upstream regulatory molecules. Examples of such regulatory proteins involved in the activation of WASP and N-WASP include: 1) the Rho-family GTPase, Cdc42; 2) the acidic lipid, phosphatidylinositol-4,5-bisphosphate (PIP₂); 3) Src family tyrosine kinases; 4) Btk and Itk tyrosine kinases; and 5) syndapin 1. See, e.g., Higgs and Pollard, supra.

Although NPFs such as the WASP/WAVE/SCAR family of proteins exhibit some structural variety and have been shown to interact with a number of different proteins, all members of this family of proteins contain at the C-terminus a hallmark domain that mediates WASP/WAVE/SCAR-stimulation of the Arp2/3 complex of proteins and nucleation of actin filaments (see FIG. 1). This C-terminal domain is referred to the VCA domain (also sometimes referred to as the WWA or simply WA region). The V region (or WH2 region) of the VCA domain is responsible for binding G-actin, whereas the CA region is responsible for binding to and activating the Arp2/3 complex (Miki, H., and Takenawa, T. (1998) Biochem. Biophys. Res. Commun. 243:73-8). Other domains shared by members of the WASP/WAVE/SCAR family are a proline rich domain (PolyPro), a basic domain (B) and an N-terminal WASP homology domain (WH1) (see FIG. 1). Upstream regulatory molecules bind to the PolyPro, B and WH1 domains to regulate the activity of the WASP/WAVE/SCAR family of proteins.

WASP and N-WASP, for example, are normally present in a folded conformation that prevents exposure of the VCA domain and inactivates the protein (Miki, H. et al. (1998) Nature 391:93-6; and Kim, A. S. et al. (2000) Nature 404:151-8). Activation occurs through two identified routes, which induce unfolding of the protein, exposure of the VCA domain and activation of Arp2/3. The first is through the binding of the Rho family GTPase Cdc42 to the CRIB domain of WASP (Miki, H. et al. (1998) Nature 391:93-6). The second is by binding of the adaptor protein Nck to the proline rich domain (Rohatgi, R. et al. (2001) J. Biol. Chem. 276:26448-52). The N-terminal WH1-domain of WASP also contributes to activity through binding of PIP₂ (Miki, H. et al. (1996) EMBO J. 15:5326-35) which anchors the protein to the cell membrane. The WH1 domain also recruits WASP-interacting protein, WIP (Ramesh, N. et al. (1997) Proc. Natl. Acad. Sci. USA 94:14671-6), which is involved in both actin polymerization and specialized activation of transcription factors such as NFAT in T cells after recruitment to WASP (Anton, I. M. et al. (2002) Immunity 16:193-204). These concerted functions of WASP in the immune system place it at the center of an essential crossroad between extracellular signaling pathways and coherent cytoskeletal responses. See also Higgs, H. N. and Pollard, T. D. (2001) Annu. Rev. Biochem. 70:649-76.

One line of evidence supporting a role for WASP/WAVE/SCAR proteins in mammalian physiology and pathology is derived from the presentation of patients suffering from Wiskott-Aldrich Syndrome. WAS patients are deficient in the eponymous protein, WASP. These patients exhibit a heterogeneous array of symptoms ranging in severity. WAS patients most commonly suffer from general immunodeficiency, thrombocytopenia, and eczema (see, e.g., Sullivan, K. E. et al. (1994) J. Pediatrics 125:876-885; Zhu, Q. et al. (1997) Blood 90:2680-2689). For example, in WAS patients NK cells display impaired cytotoxicity and there are reduced numbers of B cells and platelets. T-cells from WAS patients fail to respond to antigen presentation, and monocytes and neutrophils from WAS patients are often found to be defective in chemotaxis responses (Snapper, S. B., and Rosen, F. S. (1999) Annu. Rev. Immunol. 17: 905-929).

Mice expressing a version of WASP lacking the GBD/CRIB domain exhibit a subset of these characteristics (Snapper, S. B. et al. (1998) Immunity 9:81-91). Restriction of the WAS phenotype to haematopoietic cells is consistent with expression of WASP only in haematopoietic tissues. N-WASP is also an essential gene in mice. Targeted disruption of N-WASP causes embryonic lethality (Snapper, S. B. et al. (2001) Nat. Cell Biol. 3:897-904).

Formins are another key component in the actin nucleation machinery (see, e.g., Evangelista, M. et al. (2003) Journal of Cell Science 116:2603-2611; Kovar, D. R. and Pollard, T. D. (2004) Nature Cell Biology 6:1158-1159). Formins are conserved proteins in eukaryotes that play important roles in cytokinesis and in the formation of actin cables and stress fibers. Formins catalyze and regulate the assembly of unbranched actin filaments independently of Arp2/3. The formins are defined by formin-homology domains (FH1 and FH2 domains) in their amino acid sequence. Both the FH1 and FH2 domains are implicated in actin assembly. The proline-rich FH1 domain binds to the actin monomer binding protein profilin and in so doing delivers ATP-bound actin to the growing barbed end of the actin filament. The FH2 domain controls actin nucleation and assembly, interacting with the growing barbed end of actin filaments. Some formins also contain a FH3 domain, which determines subcellular localization. A class of formins, the Diaphanous-related formins, including mDia1 and mDia2, are regulated by interaction with the activated GTP-bound form of Rho-type GTPases, in a manner reminiscent of the activation of N-WASP by Cdc42.

In view of the important role of actin polymerization in a variety of cellular processes, there is a need for assays that can be utilized to identify agents that modulate the activity of the various components involved in the actin polymerization process (e.g., actin nucleators such as Arp2/3 and formins, NPFs such as the WASP/WAVE/SCAR family of proteins, and upstream regulators such as Cdc42 and Nck1).

### BRIEF DESCRIPTION OF THE DRAWINGS

FIG. 1 depicts the major domains in the WASP and WAVE/SCAR family of proteins. All consist of a similar organization of a distinct WASP/SCAR homology domain (WH1/SH1), a basic region (B), and a proline rich region. The actin polymerization machinery consists of one or two verprolin-homology domains (V), a central region (C) and an acidic domain (A). Interaction between the basic and acidic regions maintains the proteins in an inactive state. WASP and N-WASP also have a GTPase-binding CRIB domain in common.

FIG. 2 is a schematic representation showing the interactions where inhibition can occur in assays of the type described herein. Interactions at (1) and (2) are “on-target” because they involve the NPF (e.g., WASP or N-WASP). Interactions (3), (4) and (5) are “off target” because they do not involve the NPF.

FIGS. 3A and 3B indicate the amino acids that generally correspond with the major domains of WASP and N-WASP.

FIGS. 4A and 4B show the general structure of some of the WASP fragments that are described herein.

FIGS. 5A-5F are plots showing different types of parameters that can detected during the screening assays. FIGS. 5A and 5B are plots showing how differences in maximal velocity values can be determined as a measure of inhibition. FIGS. 5C and 5D are plots depicting how the time to half the maximum peak intensity can be calculated to obtain a polymerization parameter for a sample without inhibitor and for a sample containing inhibitor. FIGS. 5E and 5F depict polymerization parameters that are areas under a plot of signal (fluorescence) intensity as a function of time in the absence and presence of an inhibitor.

FIG. 6 depicts the extent of purification of full length WASP using certain purification methods described herein.

FIG. 7 is a chart in which fluorescence is plotted as a function of time (seconds). The chart illustrates that full length WASP (FL-WASP) and full length N-WASP (FL-N-WASP) alone can only weakly stimulate actin polymerization, but that inclusion of the activators Cdc42 or Nck1 can accelerate actin polymerization 13 times. The significant regulation of FL-WASP and FL-N-WASP obtained by methods provided herein indicates that the proteins are properly folded.

FIG. 8 is a plot comparing the relative activities of FL-WASP as compared to two truncated forms of WASP: 105-WASP (a version of WASP that lacks the WH1 domain), and the VCA/WA domain. The results shown in this plot demonstrate that FL-WASP is approximately 20 times more active than 105-WASP and 70 times more potent than the VCA domain alone.

FIG. 9 is a graph that illustrates the ability of four upstream activators (Cdc42, Nck1, Nck2 and Rac1) to activate FL-WASP. The results show that: 1) Nck1 was the most potent activator; 2) Cdc42 in the absence of PIP₂ vesicles fully activates FL-WASP; and 3) there is a bell shaped dependence between Nck1 and Nck2 and barbed end concentrations.

FIG. 10 is a graph that illustrates the ability of the four upstream activators shown in FIG. 9 to activate N-WASP. The results shown in this figure demonstrate that: 1) Rac1 activates FL N-WASP; 2) in the absence of PIP₂, Rac1 is a more potent N-WASP activator than Cdc42; 3) Nck1 and Nck2 were the only FL-N-WASP activators that can stimulate production of maximal concentration of barbed ends; 4) Nck2 is a significantly better activator of N-WASP than WASP; and 5) there is a bell shaped dose dependence curve for Nck1, Nck2 and Rac1.

FIG. 11 is a chart which illustrates the effect that PIP₂ has on the maximal rate of polymerization in the presence of FL-WASP and different upstream activators, namely Cdc42, Rac1, Nck1 and Nck2. The chart shows that: 1) PIP₂ had no effect on FL-WASP in the absence of small GTPases or Nck; and 2) PIP₂ had a strong inhibitory effect on WASP stimulated actin polymerization in the presence of both small GTPases or Nck.

FIG. 12 is a chart similar to that described in FIG. 11, except that it represents the effect of PIP₂ on the maximal rate of polymerization in the presence of N-WASP and Cdc42, Rac1, Nck1 or Nck2. The chart demonstrates that: 1) PIP₂ had a marked synergestic effect on N-WASP activation by Rac1 or Cdc42; and 2) PIP₂ inhibited Nck stimulated activation of N-WASP.

FIG. 13 is a graph that illustrates the ability of compounds, which were identified as actin polymerization inhibitors in the high throughput screening primary assay, to inhibit podosome formation in a cell-based secondary assay. The results shown in this figure demonstrate that four compounds (A, B, C and D), which were identified in the primary screening assay and characterized as Arp2/3 inhibitors in the secondary screening assays, inhibit podosome formation in a dose-dependent manner in PMA-treated THP-1 cells, as described in Example 14.

FIG. 14 is a graph that illustrates the ability of compounds, which were identified as actin polymerization inhibitors in the high throughput screening primary assay, to inhibit bacterial motility in a cell-based secondary assay. The results shown in this figure demonstrate that six compounds (1, 2, 3, 4, 5 and 6), which were identified in the primary screening assay and characterized as Arp2/3 inhibitors in the secondary screening assays, inhibit the motility of Listeria monocytogenes in a dose-dependent manner in infected SKOV-3 cells, as described in Example 15.

### DETAILED DESCRIPTION

I. Definitions

All technical and scientific terms used herein have the meaning commonly understood by a person skilled in the art to which this invention belongs, including the definitions provided herein. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); THE GLOSSARY OF GENETICS, 5TH ED., R. Rieger et al. (eds.), Springer Verlag (1991); and Hale & Marham, THE HARPER COLLINS DICTIONARY OF BIOLOGY (1991).

The terms “nucleic acid,” “polynucleotide,” and “oligonucleotide” are used interchangeably and refer to a deoxyribonucleotide or ribonucleotide polymer in either single-, double-, or triple-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties. In general, an analogue of a particular nucleotide has the same base-pairing specificity; i.e., an analogue of A will base-pair with T. The terms additionally encompass nucleic acids containing known nucleotide analogs or modified backbone residues or linkages, that are synthetic, naturally occurring, or non-naturally occurring and that have similar binding properties as the reference nucleic acid. Examples of such analogs include, without limitation, phosphorothioates, phosphoramidates, methyl phosphonates, chiral-methyl phosphonates, 2-O-methyl ribonucleotides, and peptide-nucleic acids (PNAs).

“Polypeptide” and “protein” are used interchangeably herein and include a molecular chain of amino acids linked through peptide bonds. The terms do not refer to a specific length of the product. Thus, “peptides,” “oligopeptides,” and “proteins” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide.

A “fusion protein” or “fusion polypeptide” is a molecule in which two or more protein subunits are linked, typically covalently. The subunits can be directly linked or linked via a linking segment. An exemplary fusion protein is one in which a domain from a nucleation promoting factor (e.g., VCA region) is linked to one or more purification tags (e.g., glutathione-S-transferase, His6, an epitope tag, and calmodulin binding protein).

The term “operably linked” or “operatively linked” is used with reference to a juxtaposition of two or more components (e.g., protein domains), in which the components are arranged such that each of the components function normally and allow the possibility that at least one of the components can mediate a function that is exerted upon at least one of the other components. By way of illustration, a transcriptional regulatory sequence (e.g., a promoter) is operably linked to a coding sequence if the transcriptional regulatory sequence controls the level of transcription of the coding sequence in response to the presence or absence of one or more transcriptional regulatory factors. With respect to fusion proteins or polypeptides, the terms can refer to the fact that each of the components performs the same function in the linkage to the other component as it would if it were not so linked. For example, in a fusion protein in which the VCA region of a nucleation promoting factor is fused to a glutathione-S-transferase (GST) tag, these two elements are considered to be operably linked if the VCA region can still bind to and activate Arp2/3 and the GST tag can bind to glutathione (e.g., the glutathione on a glutathione Sepharose matrix).

The terms “identical” or percent “identity,” in the context of two or more nucleic acids or polypeptides, refer to two or more sequences or subsequences that are the same or have a specified percentage of nucleotides or amino acid residues that are the same, when compared and aligned for maximum correspondence, as measured using a sequence comparison algorithm such as those described below for example, or by visual inspection.

The phrase “substantially identical” or “substantial sequence identity,” in the context of two nucleic acids or polypeptides, refers to two or more sequences or subsequences that have at least 75%, preferably at least 85%, more preferably at least 90%, 95%, 97%, 99% or higher nucleotide or amino acid residue identity, when compared and aligned for maximum correspondence, as measured using a sequence comparison algorithm such as those described below for example, or by visual inspection. Preferably, the substantial identity exists over a region of the sequences that is at least about 10, 20, 30, 40 or 50 nucleotides or amino acids in length, in some instances over a longer region such as 60, 70 or 80 nucleotides or amino acids, and in other instances over a region of at least about 100, 150, 200 or 250 nucleotides or amino acid residues. And, in still other instances, the sequences are substantially identical over the full length of the sequences being compared, such as the coding region of a nucleotide for example.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection [see generally, Current Protocols in Molecular Biology, (Ausubel, F. M. et al., eds.) John Wiley & Sons, Inc., New York (1987-1999, including supplements such as Supplement 46 (April 1999)]. Use of these programs to conduct sequence comparisons are typically conducted using the default parameters specific for each program.

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. (See Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another indication that two nucleic acid sequences are substantially identical is that the two molecules hybridize to each other under stringent conditions. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA.

A further indication that two nucleic acid sequences or polypeptides are substantially identical is that the polypeptide encoded by the first nucleic acid is immunologically cross reactive with the polypeptide encoded by the second nucleic acid, as described below.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

A polypeptide is typically substantially identical to a second polypeptide, for example, where the two peptides differ only by conservative substitutions. A “conservative substitution,” when describing a protein, refers to a change in the amino acid composition of the protein that does not substantially alter the protein's activity. Thus, “conservatively modified variations” of a particular amino acid sequence refers to amino acid substitutions of those amino acids that are not critical for protein activity or substitution of amino acids with other amino acids having similar properties (e.g., acidic, basic, positively or negatively charged, polar or non-polar, etc.) such that the substitutions of even critical amino acids do not substantially alter activity. Conservative substitution tables providing functionally similar amino acids are well-known in the art. See, e.g., Creighton (1984) Proteins, W.H. Freeman and Company. In addition, individual substitutions, additions or deletions which alter, add or delete a single amino acid or a small percentage of amino acids in an encoded sequence are also “conservatively modified variations.”

The term “stringent conditions” refers to conditions under which a probe or primer will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tₘ) for the specific sequence at a defined ionic strength and pH. In other instances, stringent conditions are chosen to be about 20° C. or 25° C. below the melting temperature of the sequence and a probe with exact or nearly exact complementarity to the target. As used herein, the melting temperature is the temperature at which a population of double-stranded nucleic acid molecules becomes half-dissociated into single strands. Methods for calculating the Tₘ of nucleic acids are well known in the art (see, e.g., Berger and Kimmel (1987) Methods in Enzymology, vol. 152: Guide to Molecular Cloning Techniques, San Diego: Academic Press, Inc., and Sambrook et al. (1989) Molecular Cloning: A Laboratory Manual, 2nd ed., vols. 1-3, Cold Spring Harbor Laboratory), both incorporated herein by reference. As indicated by standard references, a simple estimate of the Tₘ value can be calculated by the equation: Tₘ=81.5+0.41(% G+C), when a nucleic acid is in aqueous solution at 1 M NaCl (see e.g., Anderson and Young, “Quantitative Filter Hybridization,” in Nucleic Acid Hybridization (1985)). Other references include more sophisticated computations which take structural as well as sequence characteristics into account for the calculation of Tₘ. The melting temperature of a hybrid (and thus the conditions for stringent hybridization) is affected by various factors such as the length and nature (DNA, RNA, base composition) of the probe and nature of the target (DNA, RNA, base composition, present in solution or immobilized, and the like), and the concentration of salts and other components (e.g., the presence or absence of formamide, dextran sulfate, polyethylene glycol). The effects of these factors are well known and are discussed in standard references in the art, see e.g., Sambrook et al., Molecular Cloning: A Laboratory Manual, 3rd edition, Cold Spring Harbor Press, N.Y., (2001); Current Protocols in Molecular Biology, (Ausubel, F. M. et al., eds.) John Wiley & Sons, Inc., New York (1987-1993). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes or primers (e.g., 10 to 50 nucleotides) and at least about 60° C. for long probes or primers (e.g., greater than 50 nucleotides). Stringent conditions can also be achieved with the addition of destabilizing agents such as formamide.

The term “isolated,” “purified” or “substantially pure” means an object species (e.g., an Arp2/3 complex) is the predominant macromolecular species present (i.e., on a molar basis it is more abundant than any other individual species in the composition), and preferably the object species comprises at least about 50 percent (on a molar basis) of all macromolecular species present. Generally, an isolated, purified or substantially pure Arp2/3 complex or nucleic acid will comprise more than 80 to 90 percent of all macromolecular species present in a composition. Most preferably, the object species is purified to essential homogeneity (i.e., contaminant species cannot be detected in the composition by conventional detection methods), wherein the composition consists essentially of a single macromolecular species.

A “control value” or simply “control” generally refers to a value (or range of values) against which an experimental or determined value is compared. Thus, in the case of a screening assay, the control value can be a value for a control reaction that is conducted under conditions that are identical those of a test assay, except that the control reaction is conducted in the absence of a candidate agent whereas the test assay is conducted in the presence of the candidate agent. The control value can also be a statistical value (e.g., an average or mean) determined for a plurality of control assays. The control assay(s) upon which the control value is determined can be conducted contemporaneously with the test or experimental assay or can be performed prior to the test assay. Thus, the control value can be based upon contemporaneous or historical controls.

A difference between an experimental and control value can be considered to be “significant” or “statistically significant” if the difference is greater than the experimental error associated with the assay, for example. A difference can also be statistically significant if the probability of the observed difference occurring by chance (the p-value) is less than some predetermined level. As used herein a “statistically significant difference” refers, for example, to a p-value that is <0.05, preferably <0.01 and most preferably <0.001.

The term “naturally occurring” as applied to an object means that the object can be found nature.

“Modulate” can mean either an increase or decrease in the level or magnitude of an activity or process. The increase or decrease can be determined by comparing an activity (e.g., actin polymerization) under a set of test conditions as compared to the activity in a

II. Overview

Methods for assaying actin polymerization are provided. These assays can be utilized to screen diverse types of candidate agents to identify modulators of the activity of a component involved in the polymerization of globular actin (G-actin) to form filamentous actin (F-actin). The screening methods can include some or all of the following components involved in the actin polymerization process: 1) G-actin or F-actin; 2) an actin binding protein (e.g. profilin); 3) an actin nucleator/nucleation factor (e.g., Arp2/3, formins); 4) a nucleation promoting factor (e.g., WASP, N-WASP, SCAR/WAVE) that activates the actin nucleator; 5) an upstream regulator (e.g., Cdc42, Rac1, Nck) that activates the nucleation promoting factor; and 6) an actin filament severing or depolymerizing factor (e.g., cofilin, DAF, severin). The screening methods can thus be utilized to identify modulators that affect polymerization by influencing actin itself, direct or indirect modulators of the polymerization state of actin, or proteins or other cellular components whose function is naturally or artificially coupled to an actin polymerization or depolymerization reaction.

Components for conducting the assay and screening methods disclosed herein are also described. These components, for example, include purified actin nucleators (e.g., purified Arp2/3 and formins), purified nucleation promoting factors (e.g., purified WASP and N-WASP proteins) and purified upstream regulators (e.g., purified forms of Cdc42). Kits including one or more of the components are also included.

Given the important role that actin polymerization plays in a variety of cellular processes, the screening methods and kits that are provided can be utilized to identify agents that can be utilized to modulate a number of cellular activities. The role of actin polymerization in cell motility, for example, means that agents identified by the screening methods can have value as candidate agents in the treatment of metastasis of tumors and/or in the treatment of inflammatory diseases. The role of actin polymerization in platelet function, for example, means that agents identified by the screening methods can have value as candidate agents in the treatment of cardiovascular and inflammatory conditions in which it is desirable to inhibit platelet activation, adhesion and secretion of platelet contents.

III. General Screening System

The screening system utilizes an actin polymerization readout to assess whether one or more candidate agents modulate the activity of a component involved in the polymerization process. The assay is based in part in taking advantage of the fundamental role that Arp2/3 plays in the formation of branched actin filament networks and the recognition that actin polymerization pathway involves a series of regulated processes in which: 1) an upstream regulator binds a NPF to activate it; 2) the activated NPF in turn binds Arp2/3 and activates it; 3) Arp2/3 initiates nucleation of actin; and 4) G-actin is incorporated into the nucleated actin to form F-actin. The formation of F-actin can be detected in various ways but in general involves detecting a characteristic that distinguishes F-actin from G-actin.

The components included in the screening assay typically include G-actin, Arp2/3 or other nucleator protein, one or more nucleation promoting factors (NPF), and/or one or more upstream regulators. Various actin binding proteins can also be included in some assays. In some assays, ATP-actin is maintained in the unpolymerized state by keeping it on ice and at low salt. Upon addition of suitable polymerization salts, Arp2/3, and NPFs, polymerization occurs. The total rate of G-actin to F-actin conversion is linearly related to the number of filament ends and to the G-actin concentration. Since each activated Arp2/3 molecule generates one filament end, if the Arp2/3 concentration is large enough to render the number of filament ends generated independently of Arp2/3 negligible, the rate of polymerization is linearly related to the concentration of activated Arp2/3.

The screening methods generally involve combining components of an actin polymerization or depolymerization assay together in the presence of a candidate agent under conditions in which, in the absence of the candidate agent, G-actin can become incorporated into F-actin or F-actin can depolymerize and become G-actin. After the assay components have been combined, polymerization is detected over time to determine a parameter that is a measure of the extent of the polymerization of actin into F-actin. The value for the determined polymerization parameter is then optionally compared with the polymerization parameter determined for a corresponding control assay. A difference between the parameters is an indication that the candidate agent is a modulator of one of the assay components.

In some methods, the polymerization reaction is detected by including acyrolodan-labeled G-ATP-actin in the assay mixture. The fluorescence spectrum of acrylodan-actin changes on polymerization. The fluorescence of acrylodan-actin becomes more intense in F-actin solutions compared to G-actin solutions, the peak of the fluorescence spectrum shifts from 492 nm to 465 nm, and the fluorescence spectrum becomes narrower. In some methods, the polymerization reaction is detected by including pyrene-labeled G-ATP-actin in the assay mixture. The fluorescence of pyrene-actin changes on polymerization. In F-actin, the pyrene fluorescence is blueshifted and shows an altered lineshape such that the maximum of the F-G difference spectrum occurs at 407 nm but the G-actin fluorescence is more intense at wavelengths above ˜430 nm. Other methods, however, utilize dyes that exhibit considerable fluorescence enhancement in F-actin solutions as compared to G-actin solutions (e.g., the fluorescent dye 4-(dicyano)julolidine (DCVJ)).

Since Arp2/3 activation involves a variety of signaling molecules, there are a corresponding variety of biochemical screens that can be assembled that utilize Arp2/3-mediated actin polymerization as the readout. Examples of sources of actin, and types of nucleation factors, NPFs and upstream regulators that can be incorporated into an assay are listed in Tables 1 and 2. These components can be mixed together in a variety of combinations. Some assays, for instance, are conducted by combining actin, an actin nucleator and a NPF from Tables 1 and 2. Other assays also include an upstream regulator and/or actin binding protein as listed in these two tables.

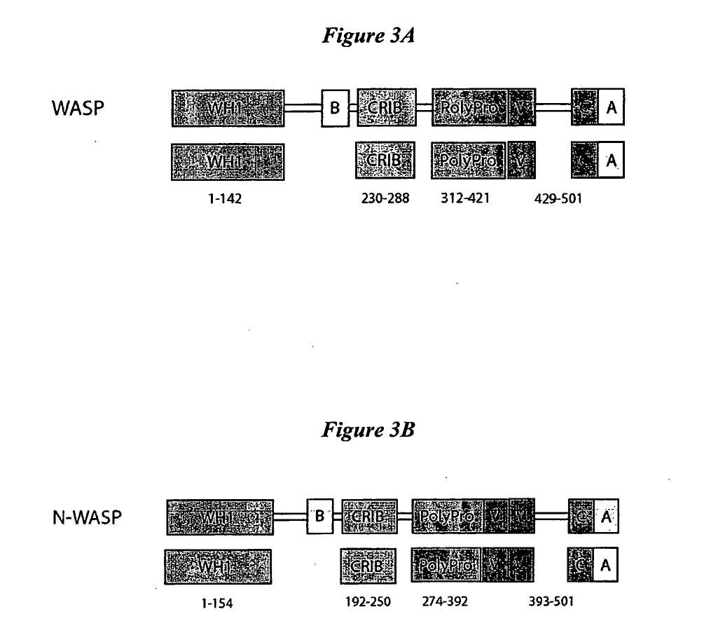

Using various combinations of the components listed in Tables 1 and 2, the screening assays can be utilized to identify candidate agents that modulate actin polymerization at any of the steps along the activation pathway. Using fluorescence based assays as an example, any candidate agent that modulates (e.g., inhibits) fluorescence can be considered “a hit.” These hits can be of two major types: 1) “on target hits,” which refer to candidate agents that interact with a NPF; and 2) “off target hits,” which refer to candidate agents that interact with some other component of the assay besides the NPF. As shown in FIG. 2, for instance, some off-target hits can arise from candidate agents inhibiting an upstream regulator such as Cdc42 (hit 3), Arp2/3 (hit 4) and actin polymerization itself (hit 5). On-target hits can arise, for example, from inhibition of WASP unfolding to expose the VCA domain (hit 1) or inhibiting Arp2/3 (or actin monomer) binding to the VCA domain itself (hit 2). Some inhibitors are ones that stabilize the autoinhibited conformation of WASP, thereby preventing exposure of the VCA domain. By omitting other mediators of WASP function from the assay (e.g., one of the many adaptors and kinases that interact with WASP), hits will only select candidate agents that target the initiation of actin polymerization.

As described in greater detail below, once a compound is identified as modulating actin polymerization, a series of secondary screens can be conducted to determine with which component(s) of the assay (e.g., actin, nucleation factor, NPF and/or upstream regulator) the candidate agent interacts.

IV. Assay Components

A. Actin

Various types of actin can be utilized in the screening process. As noted above, some assays utilize acrylodan-labeled G-actin and monitor its incorporation into F-actin. Acrylodan-labeled-G-actin can be prepared as described, for example, by Marriott et al. (Biochemistry 27:6214-6220, 1988). Some assays utilize pyrene-labeled-G-actin and monitor its incorporation into F-actin. Pyrene-labeled G-actin can prepared as described, for example, by Kouyama and Mihashi (Eur. J. Biochem. 114:33-38, 1981) and Cooper et al. (J. Muscle Res. Cell Motil. 4:253-262, 1983). It can also be purchased from Cytoskeleton, Inc. In assays in which F-actin is detected by labeling with dyes that exhibit differential fluorescence characteristics when bound to G-actin versus F-actin, various types of unlabeled actin can be utilized. Such assays, for example, can simply contain G-actin or G-actin plus F-actin seeds. As used herein, “F-actin seeds” refers to pre-polymerized actin. G-actin is commercially available from Cytoskeleton, Inc. or can be prepared as discussed by Pardee and Spudich (1982) Methods of Cell Biol. 24:271-89, which is then usually gel filtered as discussed by MacLean-Fletcher and Pollard (1980) Biochem. Biophys. Res. Commun. 96:18-27. The actin that is utilized can be from essentially any source, including but not limited to, chicken, bovine, rabbit and porcine. The actin concentration in the final assay mixture typically is about 2-4 μM, such as about 3 μM.

It can be useful to purify actin preparations before use to obtain a purified actin that gives greater consistency in polymerization performance. One purification approach is to pass an actin preparation through a gel filtration column (e.g., G-100) and collect the actin from the trailing edge, thereby collecting lower molecular weight forms of actin. In some assays it is beneficial to use actin preparations that have not been stored form more than 2-6 weeks at −80° C. Typically, thawed actin preparations are stable for at least one day if stored on ice.

B. Actin Nucleators

The actin nucleator that is included in the assay mixture is in general an agent that can initiate nucleation of actin polymerization from free monomers. An actin nucleator includes full-length naturally occurring proteins that can initiate nucleation, fragments that have nucleation activity and variants that have substantial sequence identity with the full length proteins or fragments and that have actin nucleation activity. The term “nucleation” as used herein thus refers to the initiation of actin polymerization from free actin monomers. The concentration of the actin nucleator in the final assay mixture can vary but in general ranges from about 5-15 nM, such as 10 nM.

The Arp2/3 complex is an exemplary actin nucleator. As used herein, the term “Arp2/3 complex” (or simply Arp2/3) includes its general meaning in the art and includes Arp2/3 from essentially any source (e.g., human, amoeba and budding yeast) that has actin nucleating activity. The term thus refers, for example, to the complex of six subunits in Saccharomyces cerevisiae and seven subunits in Acanthaemoeba castellanii and humans that can nucleate new actin filaments and cross-link newly formed filaments into Y-branched actin filament arrays. Additional details regarding the nomenclature and composition of Arp2/3 complexes from non-human sources are provided in Higgs and Pollard (Ann. Rev. Biochem. 70:649-76, 2001) and in Welch and Mullins (Annu. Rev. Cell Dev. Biol. 18:247-88, 2002). The term Arp2/3 as used herein encompasses complexes in which one, some or all of the subunits is/are fragments that retain activity, or a variant with substantial sequence identity to a full length sequence or fragment that also has nucleation activity.

As shown in Tables 1 and 2, various other actin nucleators can be used in some assays. Examples of such nucleators include, but are not limited to, members of the formin family such as Candida albicans FOR1 or FHOS (GenBank Accession No. Q9Y613), VASP/Ena (GenBank Accession No. PO₅₀₅₅₂) or Mena.

The actin nucleator (e.g., Arp2/3, formin) that is included in the assays can be a purified form. The purity of the nucleator in the sample that is introduced into the assay is in some instances at least 70, 75, 80, 85, 90, 95, 97 or 99% pure.

One method for obtaining purified Arp2/3 is described in Example 1. In general, however, the purification procedure involves four primary steps. First, a sample is provided that includes Arp2/3 complex. This can be done by lysing cells such as human platelets that contain relatively high amounts of the Arp2/3 complex. Second, the sample containing Arp2/3 complex is loaded onto a first anion exchange column (e.g., DEAE or equivalent) under conditions in which some contaminating proteins bind to the exchanger, but the complex does not. Third, the eluate from the first anion exchange column is applied to a second anion exchange column (e.g., Q-Sepharose or equivalent), wherein the Arp2/3 complex is initially bound and then eluted from the column using a salt gradient. Finally, the active fractions collected from the second ion exchange column are applied to an affinity column matrix under conditions in which Arp2/3 is bound. The affinity ligand typically includes a fragment/domain of a NPF (e.g., a CA or VCA domain) that can bind the complex. It is subsequently eluted after first eluting contaminating proteins. The first and second ion exchange columns can be run in an automated and continuous process in which eluate from the first column is applied directly to the second. Further details regarding methods for purifying Arp2/3 are provided in U.S. Provisional Patent Application No. 60/578,969, filed Jun. 10, 2004, which is incorporated herein by reference in its entirety for all purposes. Other methods for purifying Arp2/3 are discussed for example by Welch et al. (1997) Nature 385:265-269; Welch and Mitchison (1998) Methods in Enzymology 298:52-61; and Dayel et al. (2001) Proc. Natl. Acad. Sci. USA 98:14871-14876.

C. Nucleation Promoting Factor (NPF)

1. General

A number of different NPFs can be utilized in the assays. As used herein, the term “nucleation promoting factor” includes its normal meaning in the art (see, e.g., Welch and Mullins (2002) Annu. Rev. Cell Dev. Biol. 18:247-288). The term in general refers to an agent that can activate the nucleation activity of an actin nucleator (e.g., Arp2/3). Protein NPFs can be full length proteins, a domain/fragment of a full length protein that retains the capacity to activate actin nucleators, or a variant that has substantial sequence identity to the full length proteins or domains/fragments and that can activate actin nucleators.

There are a number of NPFs that can be utilized in the assay (see, e.g., Tables 1 and 2). Examples of suitable NPFs include, but are not limited to, (1) WASP, (2) N-WASP, (3) the SCAR/WAVE family of proteins (SCAR1/WAVE1, SCAR2/WAVE2, and SCAR3/WAVE3) and (4) Act A protein from Listeria monocytogenes (see also, Welch and Mullins (2002) Annu. Rev. Cell Dev. Biol. 18:247-88; and Higgs and Pollard (2001) Ann. Rev. Biochem. 70:649-76). GenBank accession numbers for the protein sequences of these proteins are listed in Table 3. This table also lists SEQ ID NOs: that provide exemplary amino acid sequences for these NPFs. The concentration of the NPF in the final assay mixture for some assays ranges from about 1-500 nM.

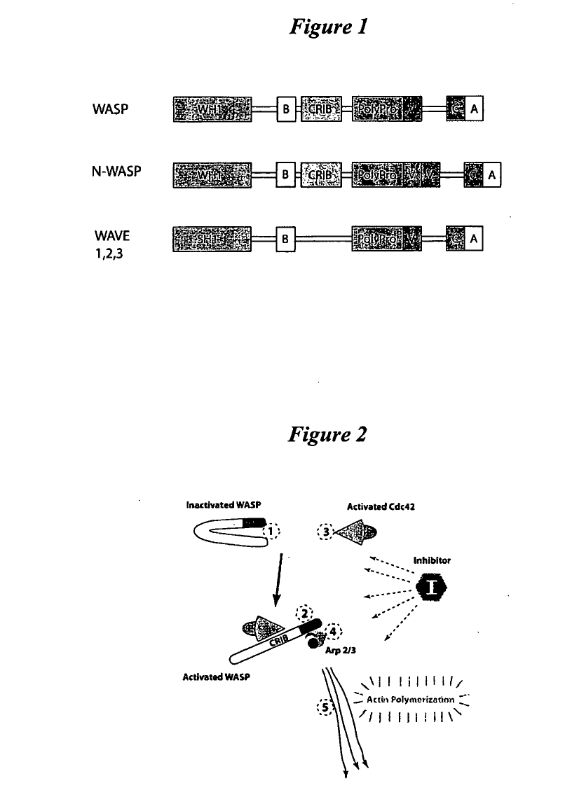

Some assays are conducted with a WASP, N-WASP or SCAR/WAVE protein. As noted above, the WASP/N-WASP/SCAR (WAVE) family of proteins share a number of domains, including 1) the VCA domain that binds G-actin and activates the Arp2/3 complex, 2) a proline rich domain (the PolyPro domain), 3) a basic domain (B), and 4) an N-terminal WASP homology domain (WH1) (see FIG. 1).

The terms “WASP protein,” “N-WASP protein,” and “SCAR protein” (or “WAVE protein”) as used herein refer respectively to a protein having an amino acid sequence of a naturally occurring WASP, N-WASP or SCAR protein, as well as variants and modified forms regardless of origin or mode of preparation. The WASP or N-WASP protein can be from various sources, including for example, various mammalian and non-mammalian sources.

The terms “SCAR protein” and “WAVE protein” are used interchangeably because both are used in the literature. In general, a reference to a SCAR protein includes the different forms of SCAR/WAVE, namely SCAR1/WAVE1, SCAR2/WAVE2 and SCAR3/WAVE3. A naturally occurring or native WASP, N-WASP or SCAR protein is a protein having the same amino acid sequence as a WASP, N-WASP or SCAR protein as obtained from nature, respectively. Native sequence WASP, N-WASP and SCAR proteins specifically encompass naturally occurring truncated or soluble forms, naturally occurring variant forms (e.g., alternatively spliced forms), naturally occurring allelic variants, and forms including postranslational modifications of WASP, N-WASP, and SCAR, respectively. Specific examples of native amino acid sequences for WASP, N-WASP and the SCAR family (e.g., SCAR1/WAVE1, SCAR2/WAVE2, and SCAR3/WAVE3) are provided in Table 3, together with an exemplary nucleic acid sequence that encodes for these proteins.

The term “variant” or “analogue” generally refers to proteins that are functional equivalents to a native sequence that have similar amino acid sequences and retain, to some extent, one of the activities of the corresponding native protein. Variants/analogues include fragments that retain one or more activities of the corresponding native protein. Examples of WASP and N-WASP activity include, but are not limited to, capacity to: 1) bind Arp2/3 and actin; 2) activate the actin nucleation activity of Arp2/3 (descriptions of assays to detect nucleation activity are provided below); 3) bind an upstream regulator; 4) be regulated by one or more upstream regulators, thereby rendering the WASP or N-WASP protein able to activate the nucleation activity of Arp2/3; and 5) bind downstream regulators initiating signal transduction cascades. Some of the WASP and N-WASP proteins that are provided are able to recapitulate the full activity of WASP or N-WASP, which means that these proteins have all five of the activities just listed.

Variants and analogues also include proteins that have substantial sequence identity to a corresponding native sequence. Such variants include proteins having amino acid alterations such as deletions, insertions and/or substitutions. Typically, such alterations are conservative in nature such that the activity of the variant protein is substantially similar to a native sequence WASP, N-WASP or SCAR protein (see, e.g., Creighton (1984) Proteins, W.H. Freeman and Company). In the case of substitutions, the amino acid replacing another amino acid usually has similar structural and/or chemical properties.

Variants of WASP, N-WASP and SCAR also include modified proteins in which one or more amino acids of a native sequence WASP, N-WASP or SCAR, respectively, have been altered to a non-naturally occurring amino acid residue. Such modifications can occur during or after translation and include, but are not limited to, phosphorylation, glycosylation, cross-linking, acylation and proteolytic cleavage. Variants also include modified forms in which the protein includes modified protein backbones (e.g., glycosylation, carboxylations, acetylations, ubiquitinization and phosphorylation).

The WASP, N-WASP and SCAR proteins can be both deletion mutants, in which one or more domains have been at least partially deleted, and fusion proteins that can include: 1) a full length WASP, N-WASP or SCAR domain or a domain corresponding to the deletion mutant, and 2) one or more tags. The deletion mutants can vary in size, but in some instances are less than 450, 400, 350, 300, 250, 200, 150 or 100 amino acids in length. Typically, the deletion mutants are at least 50, 60, 70 or 80 amino acids in length.

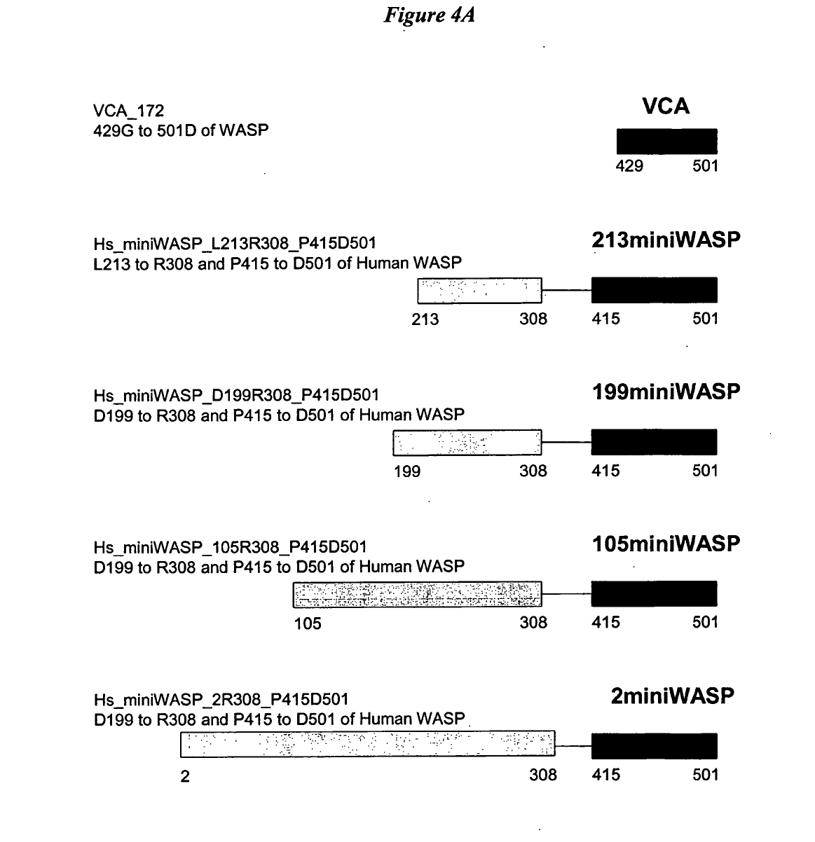

With respect to WASP and N-WASP, FIGS. 3A and 3B indicate the general organization of the major WASP and N-WASP domains with respect to one another and provides an indication of the approximate boundaries of each of the domains with respect to a full-length WASP sequence (SEQ ID NO:2) and with respect to a full-length N-WASP sequence (SEQ ID NO:4). See also Yarar, D. (2002) Molecular Biology of the Cell 13:4045-59, and Hufner, K. et al. (2001) J. Biol. Chem. 276:35761-7. Table 3 also summarizes the general boundaries of the domains for WASP and N-WASP, as well as for SCAR1, SCAR2 and SCAR3.

It should be recognized, however, that the regions as defined in FIGS. 3A and 3B and Table 3 are approximate and that the regions can extend or omit a limited number of amino acids from the amino or carboxyl end of each domain. For the smaller domains such as the B, CRIB and VCA domains, for instance, the regions can extend or omit about 1, 2, 3, or 4 amino acids from one or both of the amino and carboxyl ends. For the larger domains such as the WH1 domain and the PolyPro domain, the regions can extend or omit about 1-10 amino acids (e.g., 1, 2, 4, 6, 8 or 10) from the amino or carboxyl ends.

2. Deletion Mutants

Because the VCA region can bind actin and activate Arp2/3, the NPF in some assays are deletion mutants or analogues of WASP or N-WASP in which the WH1 domain, B domain, CRIB domain, and PolyPro domain of WASP, N-WASP are deleted. The assays can also be conducted with deletion mutants of SCAR in which the WH1 domain, B domain, and PolyPro domain are deleted. A group of proteins in this particular group of deletion mutants are those that include primarily the VCA region. Specific examples of such deletion mutants are proteins that include just the VCA region of WASP, N-WASP or SCAR as listed in Table 3.

A second set of NPF proteins are deletion mutants that include the VCA region from WASP, N-WASP or SCAR/WAVE but in which one or more of the other domains has been disabled. The term “disabled” as used herein means that a sufficient portion of the region has been deleted or otherwise affected such that the region no longer maintains one or more of its normal activities. In some instances, the entire region encoding the domain is deleted.

One subgroup of such proteins are those in which some or all of the WH1 region and the PolyPro region from WASP, N-WASP or SCAR/WAVE have been removed. The WH1 region generally corresponds approximately to amino acids 1-140 or 1-150 of the full length sequences of WASP, N-WASP, SCAR1/WAVE1, SCAR2/WAVE2 or SCAR3/WAVE3. The region is indicated more specifically for each of these proteins in Table 3. The approximate region corresponding to the PolyPro segment of WASP, N-WASP, and the SCAR/WAVE protein family are also listed in Table 3.

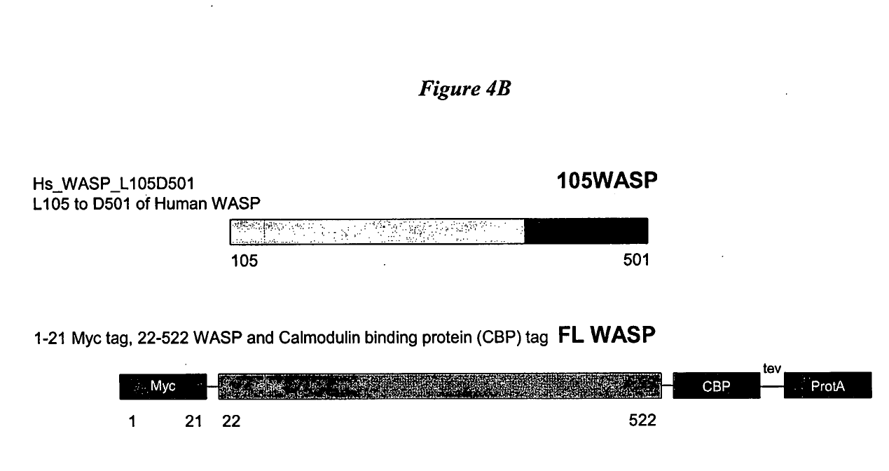

Specific examples of NPF proteins lacking at least a part or all of the WH1 and PolyPro regions that can be utilized in certain assays include, but are not limited to, 213miniWASP, 199miniWASP and 105miniWASP (see FIGS. 4A and 4B). These three proteins each lack all or some of the WH1 region and the entire PolyPro region (approximately residues 309-414 of SEQ ID NO:2). 213miniWASP thus includes, for example, amino acid residues 213-308 and 415-501 from the full length WASP sequence SEQ ID NO:2). 199miniWASP includes residues 199-308 and 415-501 of SEQ ID NO:2. 105miniWASP includes residues 105-308 and 415-501 of SEQ ID NO:2. 213miniWASP and 199miniWASP are regulated by Cdc42 (i.e., Cdc42 can bind and activate the construct so the activated construct can in turn activate the nucleation activity of Arp2/3).

A specific example of a NPF protein that lacks only a portion of WH1, but still lacks the PolyPro region, is 2miniWASP. This particular protein includes residues 2-308 and 415-501 of SEQ ID NO:2.

A third class of NPF proteins that can be utilized in some assays include WASP, N-WASP and SCAR proteins that include the WH1 domain but in which the PolyPro region is (e.g., deleted). A fourth class of NPF proteins that can be utilized in some assays are WASP, N-WASP and SCAR deletion mutants in which the PolyPro region is maintained but an N-terminal region (e.g., WH1 domain) is at least partially removed. Some proteins in this class are ones that include the B, CRIB/GBD (WASP and N-WASP), PolyPro and VCA domains, but in which some or all of the WH1 has been removed. One specific example is 98N-WASP, which includes amino acids 98-501 of SEQ ID NO:4. Another specific example is the 105WASP protein, which includes amino acids 105-501 of SEQ ID NO:2 (see FIG. 4B). These particular proteins are of interest because they fully recapitulate the activity of full-length length WASP in that they are regulated by Cdc42, PIP₂, Nck1, and Rc1. They can also activate actin nucleation by Arp2/3.

The proteins that are provided also include variants of the foregoing four classes of proteins that have substantial sequence identity to the proteins in these classes and that retain some or all of the same activities. The WASP and N-WASP proteins that are provided thus include, for example, proteins that have substantial sequence identity with SEQ ID NOs:2, 4, 6, 8, 10, 12 and that retain the activity of the corresponding protein as listed in Table 4.

The various deletion mutants that can be utilized in the assays can be prepared based upon the sequence information that is provided herein (see, e.g., Table 3) and recombinant technologies such as described, for example, in Sambrook et al., Molecular Cloning: A Laboratory Manual, 3rd ed., Cold Spring Harbor Press, N.Y. (2001); and Current Protocols in Molecular Biology (Ausubel, F. M., et al. eds.), John Wiley & Sons, Inc., New York (1987-1993), which are incorporated herein by reference in their entirety for all purposes.

3. Fusion Proteins

The NPF proteins that are utilized in some assays can be fusion proteins. Such fusion proteins include, for example: 1) a WASP, N-WASP or SCAR domain, which can be a full length WASP, N-WASP or SCAR sequence (see Table 3), or an analogue/deletion mutant such as described above (see, e.g., Table 4); and 2) one or more tag domains linked or fused to the amino and/or carboxyl terminal ends of the WASP, N-WASP or SCAR protein domain. The fusion proteins thus also include fusion proteins that result from the removal of a tag from either the amino or carboxy terminus of a fusion protein that initially included a tag at each end. Some of the fusion proteins are of interest because they have the same activities of naturally occurring WASP, N-WASP or SCAR.

The tags that are incorporated into the fusion can be utilized to improve expression, to improve solubility and/or to aid in purification. The WASP, N-WASP or SCAR domain can also be a protein that has substantial sequence identity to full-length WASP, N-WASP or SCAR, or the various deletion mutants listed above. Thus, for example, the WASP, N-WASP or SCAR domain can have substantial sequence identity to the sequences provided in Table 3.

A variety of tags can be utilized, including but are not limited to: 1) a glutathione S-transferase (GST) tag, which can be used to bind to glutathione-agarose; 2) a His6 tag (or simply a HIS tag), which can be used to bind to immobilized metal-ion columns (e.g., nickel); 3) a calmodulin-binding peptide (CBP) tag that binds calmodulin-agarose columns; 4) an epitope tag (e.g., a haemagglutinin tag, a myc tag, or a FLAG tag), which can be used to bind an antibody with specific binding affinity for the epitope tag; 5) a maltose-binding protein (MBP) tag, which increases the solubility of fused proteins; and 6) a TAP tag, which the current inventors have determined can be utilized to facilitate expression of WASP and N-WASP proteins and to improve their solubility. These tags can also be used in combination, with one or more tags fused to the amino terminus and one or more additional tags fused to the carboxyl terminus.

Many of these tags are commercially available. For example, vectors useful for incorporating HIS tags in mammalian cells include vectors pcDNA3.1/Myc-His and pcDNA3.1/V5-His, which are available from Invitrogen (Carlsbad, Calif.). Vectors pBlueBacHis and Gibco (Gaithersburg, Md.) vectors pFastBacHT are suitable for expression in insect cells. HIS tags and their use with metal chelate affinity ligands such as nitrilo-tri-acetic acid (NTA) that can bind the poly histidine tag are discussed, for example, by Hochuli (“Purification of recombinant proteins with metal chelating adsorbents” in Genetic Engineering: Principles and Methods, J. K. Setlow, Ed., Plenum Press, NY, 1990). Systems for incorporating HIS tags are available from Qiagen. FLAG tags are discussed by, for example, Chubet and Brizzard (Biotechniques 20:136-141, 1996), and Knappik and Pluckthun (Biotechniques 17:754-761, 1994). Systems for fusing a GST tag are available, for example, from Promega. New England Biolabs provides systems for incorporating MBP tags. CBP systems can be obtained from Strategene. FLAG tags to a protein are available from various sources, including Kodak, Rochester N.Y.

Tags such as these can optionally be linked to segments that include protease cleavage sites to facilitate removal of the purification tag and to simultaneously elute the proteins. An example are fusion proteins in which the WASP or N-WASP protein is linked to a tag via a linker that includes a protease cleavage site such as the tobacco etch virus (TEV) protease site. The tag can be used to bind to a column that includes an appropriate ligand to bind the tag. The bound fusion protein can subsequently be released by exposing the column to a highly specific TEV protease. Further details regarding such a strategy are described in Example 6. See also, Carrington and Dougherty (1988) Proc. Natl. Acad. Sci. USA 85: 3391-3395; Dougherty et al. (1989) Virology 171: 356-364; Dougherty and Semler (1993) Microbiol. Rev. 57: 781-822; Herskovits, et al. (2001) EMBO Reports 2:1040-1046; Ehrmann et al. Proc. Natl. Acad. Sci. USA 94:13111-13115; Faber et al. (2001) J. Biol. Chem. 276: 36501-36507; Smith and Kohorn (1991) Proc. Natl. Acad. Sci. USA. 88: 5159-5162; Kapust et al. (2001) Protein Eng. 14:993-1000; and Melcher (2000) Anal Biochem 277:109-120.

One specific example is a TAP tag that includes a TEV cleavable site. TAP tags generally include an IgG-binding unit from Protein A of Staphyloccoccus (ProtA) and a binding unit from Calmodulin Binding Peptide (CBP). Certain TAP tags that are useful for fusing to the C-terminus of a protein are part of a construct that encodes for CBP, a TEV cleavage site and ProtA. Strategies for using TAP tags in certain applications are discussed, for instance, by Rigaut et al. (Nature Biotechnology 17:1030-1032, 1999) and Puig et al. (Yeast 14:1139-1146, 1998), both of which are incorporated herein by reference in its entirety for all purposes.

A number of specific examples of fusion proteins that can be utilized in various assays are provided in Table 4. As indicated in this table, specific examples of fusion proteins that can be utilized include those that include a segment that encodes full-length WASP or N-WASP, including: Myc-WASP-TAP (SEQ ID NO:14); and Myc-N-WASP-TAP (SEQ ID NO: 16). Examples of fusion proteins that include a WASP or N-WASP deletion mutant domain include: GST-105WASP (SEQ ID NO:18); Myc-105WASP-TAP (SEQ ID NO:20); GST-tev-98N-WASP (SEQ ID NO:22); and Myc-98N-WASP-TAP (SEQ ID NO:24). The TAP tag in these particular fusion proteins has the general structure CBP-tev-ProtA (SEQ ID NO:36). Fusion proteins such as those listed in Table 4 can in some instances be utilized directly, or after one or both of the C- and N-terminal tags are removed. For example, the fusion proteins listed in Table 4 as having a TAP tag can be used once the TAP tag has been cleaved off and/or after the C-terminal tag has been removed.

Details regarding the preparation of some of the full length NPF proteins, miniWASPs and other WASP fragments that are fused to tags are provided in Examples 2-3 and 5-6. Further details are provided in U.S. Provisional Application No. 60/578,913, filed Jun. 10, 2004. Other fusion proteins containing one or more tags can be prepared using conventional molecular biological techniques such as described in Sambrook et al., Molecular Cloning: A Laboratory Manual, 3rd ed., Cold Spring Harbor Press, N.Y. (2001); and Current Protocols in Molecular Biology (Ausubel, F. M., et al. eds.), John Wiley & Sons, Inc., New York (1987-1993), which are incorporated herein by reference in their entirety for all purposes.

The NPF protein that is utilized can be a high purity form. The purity of the NPF protein in the sample that is introduced into the assay is in some instances at least 70, 75, 80, 85, 90, 95, 97 or 99% pure.

D. Upstream Regulators

A number of different upstream regulators can be included in the assays. The term “upstream regulator” as used herein includes its general meaning in the art and refers generally to an agent (protein or non-protein) that can activate the activity of a NPF so it in turn can activate an actin nucleator such as Arp2/3. Examples of upstream regulators include, but are not limited to, those listed in Tables 1 and 2. The upstream regulator, if a protein, can be a full length naturally occurring protein, a fragment thereof that retains its ability to activate a NPF, or a variant that has substantial sequence similarity to a full length protein or fragment and that can activate a NPF. One or more upstream regulators can be included in the assay. One pairing, for example, is Cdc42 and PIP₂. The concentration of the upstream regulator(s) in the final assay tends to range from about 0.1-0.2 μM.

Some upstream regulators are fusion proteins that include: 1) an activation domain that can bind to a target NPF and activate it; and 2) a tag. The tag can be selected from any of these described in the section on NPF. Here, too, the tags can be utilized to improve expression, to improve solubility and to aid in purification. One or more tags can be fused to the amino and/or carboxyl terminal end of the activation domain. Some fusion proteins include the full length sequence of a naturally occurring upstream regulator and one or more tags. Other fusion proteins include a fragment of the full length sequence that retains activity.

One class of upstream regulators are GTPases (e.g., Cdc42 and Rac1). In the normal activation process for this class of regulator, the GDP bound by the GTPase must be displaced with GTP before the GTPase can activate the NPF (e.g., WASP). In assays utilizing a GTPase, the GTPase can be included in one of three different forms: 1) the wild type form, in which case the assay typically includes GTP; 2) a “dominant negative” mutant in which GDP remains bound; and 3) a constitutively active form in which GTP remains bound and cannot be hydrolyzed.

Dominant negative mutants of the Rho family GTPase, like Cdc42 and Rac1, simulate inhibition of WASP/N-WASP and WAVE2, (see, e.g., Nobes, C. D., and Hall, A. (1995) Cell 81:53-62). Inhibitory constructs generally consist of the full length protein carrying mutations or deletions within the conserved VCA domain (Miki, H. et al. (1998a) Nature 391:93-96; Miki, H. et al. (1998b) Embo J 17:6932-6941). The proline-rich domain of SCAR/WAVE2 can also be used as a dominant negative mutant (see, e.g., Miki, H. et al. (2000) Nature 408:732-735).

Example 5 provides details regarding the preparation and purification of a GST-tev-SEQ Cdc42 (SEQ ID NO:26), GST-tev-RhoC (SEQ ID NO:28), GST-tev-RhoA (SEQ ID NO:30), GST-tev-Rac1 (SEQ ID NO:32), GST-tev-Nck1 (SEQ ID NO:34) and GST-Nck2 (SEQ ID NO:46). Similar fusion proteins can be prepared using related approaches with other upstream regulators and other tags. Fusion proteins such as these are sometimes used with the tag, and in other instances after the tag has been removed.

Not all upstream regulators are proteins. Other upstream regulators that can be included in the assay mixture are PIP₂, PC (phosphatidyl choline), PS (phosphatidyl serine) and PE (phosphatidyl ethanolamine). These can be in the form of vesicles. If included, the concentration in the assay is generally 5-30 μM.

E. Actin Binding Proteins

Various proteins bind actin and have various roles (e.g., stabilizers). Such proteins can also be included in the assays to identify agents that modulate their activity and/or to study their interactions with actin. Examples of such proteins include, but are not limited to those listed in Tables 2 and 3.

F. Other Assay Components

Those of skill will appreciate that a number of other assay components can be included in the assay mixture including, for instance: 1) a buffer; 2) reducing agents to keep proteins in a reduced state (e.g., dithiothreitol (DTT)); 3) metal chelators (e.g., EDTA, EGTA); 4) an antifoaming agent or surfactant to minimize the foam generated during processing of the assay mixture; and 5) polymerization salts that promote actin polymerization.

A variety of different antifoaming agents can be utilized. Suitable agents include, but are not limited to, antifoam 289 (Sigma), and others that are commercially available. Suitable surfactants include, but are not limited to, Tween, Tritons including Triton X-100, saponins, and polyoxyethylene ethers. This minimizes bubble formation which often results in conventional methods requiring pipetting into low volume assay wells. Generally the antifoam agents, detergents or surfactants are added at a range from about 0.01 ppm to about 10 ppm, with from about 1 to about 2 ppm being preferred.

“Polymerization salts” as used herein generally refers to metal ion salts that promote actin polymerization. The metal ion, for example, can be a cofactor for a protein in the assay. A typical polymerization salt composition contains a magnesium ion salt (e.g., magnesium chloride) and a potassium ion salt (e.g., potassium chloride). The concentration of the various ions from the polymerization salt in the final assay composition is typically about 0.5-1.5 mM magnesium ion (e.g., about 0.8 mM) and 20-100 mM postassium ion.

V. Combining Assay Components

The assay components can be combined in a variety of ways using conventional assay apparatus and automated. One approach that is designed to be compatible with high throughput screening and designed to minimize the effects of pipetting errors, involves formulating the assay as a two-component mix. This two-component formulation is also chosen to prevent actin from polymerizing prematurely and to prevent the functionally important interactions that are to be interrogated from pre-forming. Instead, the interactions are formed de novo in the presence of the potential modulatory agent.

In assays conducted utilizing the two-component approach, the major components in one mixture (Mix 1) are a G-buffer solution (including buffer and ATP), actin, acryolodan-actin or pyrene-actin, and an upstream regulator (e.g., Cdc42). The other mixture (Mix 2) contains G-buffer, a NPF (e.g., WASP), and an actin nucleator (e.g., Arp2/3). The G-buffer and Mix 2 are typically kept on ice during preparation of the final assay mixture. This helps minimize premature actin polymerization. The ATP is generally added to the G-buffer in the form of a fresh powder rather than as a pre-made solution. Mixes 1 and 2 can both include an antifoam agent to minimize foaming during mixing. Mix 2 also typically includes polymerization salts (e.g., 400 mM KCl, 8 mM MgCl₂, 1× G-buffer without the DTT).

The two mixes can be mixed with the candidate agent in a variety of different formats. One approach that is suitable for high throughput screening is to place a sample of a candidate agent (or a mixture of candidate agents) in each of a plurality of wells in a multi-well plate and then add a sample from each of Mix 1 and Mix 2 into each of the wells. The resulting assay mixture can then be mixed (e.g., by shaking the multi-well plate). Each of these steps can be automated. Further details regarding high throughput screening (HTS) methods are described below.

Some assays are used to identify agents that stabilize the folded auto-inhibited form of WASP or that inhibit the ability of the actin nucleator (e.g., Arp2/3) to initiate actin nucleation. In assays of this type, the concentration of WASP and Arp2/3 should be at rate limiting levels (e.g., a two-fold decrease in WASP or Arp2/3 concentration should result in at least a two-fold decrease in the maximal actin polymerization rate).

VI. Detection of Actin Polymerization

Some assays are conducted using acrylodan-labeled G-actin or pyrene-labeled G-actin. As noted above, the fluorescence spectrum of both acrylodan-actin and pyrene-actin changes on polymerization. In particular, the fluorescence of acrylodan-actin becomes more intense in F-actin solutions as compared to G-actin solutions, the peak of fluorescence spectrum shifts from 492 nm to 465 nm, and the fluorescence spectrum becomes narrower. Pyrene fluorescence is blueshifted in F-actin and shows an altered lineshape such that the maximum of the F-G difference spectrum occurs at 407 nm, whereas G-actin fluorescence is more intense at wavelengths above ˜430 nm. Acrylodan-labeled G-actin can be prepared as described by, e.g., Marriott et al. (1988) Biochemistry 27:6214-6220. Pyrene-labeled-G-actin is commercially available. It can also be prepared as discussed by, e.g., Kouyama et al. (1981) Eur. J. Biochem. 114:33-38).

In a different approach, an agent that gives a differential signal when bound to polymerized F-actin as compared to G-actin is added to the assay solution. Some methods of this general type, for instance, use dyes that fluoresce considerably more strongly in F-actin solutions as compared to G-actin solutions. One example of such a dye is the fluorescent dye 4-(dicyano)julolidine (DCVJ).

VII. Data Analysis

In general, a signal associated with polymerization is detected over time. Recording signal formation as actin polymerizes typically yields a sigmoidal curve. This is because initially there is a lag phase. This lag phase is typically followed by a relatively rapid increase in signal as the nucleated actin begins to polymerize. Eventually the signal reaches a plateau once most of the G-actin has become incorporated into F-actin. When the assay utilizes acrylodan-labeled actin or pyrene-labeled actin, the signal that is detected over time is a fluorescence signal.

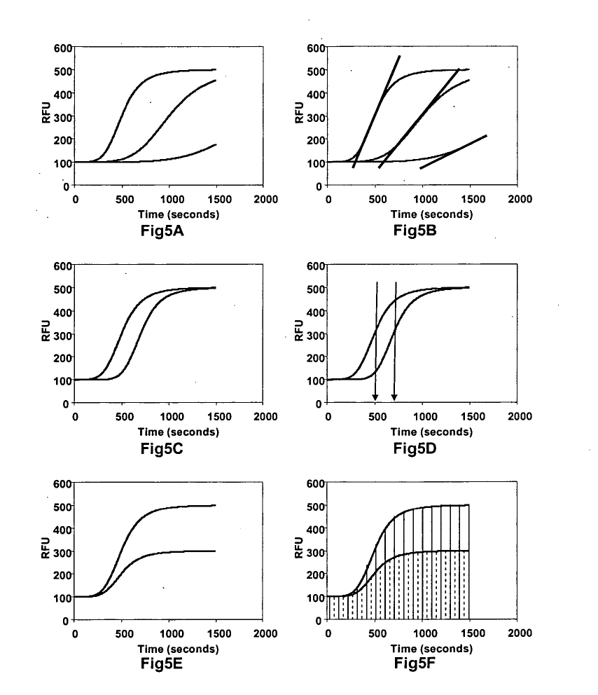

The data obtained during the polymerization process can be analyzed in various ways. In general, a parameter is determined that is a measure of the extent of polymerization. During some methods, the change in signal (e.g., change in fluorescence) over time is recorded or plotted. A variety of different parameters can be determined from the recorded data or plot including, but not limited to: 1) maximal velocity; 2) time to half the maximum signal intensity; 3) area under a curve in which signal intensity is plotted versus time; and 4) a measure of the slope of the polymerization curve over some time interval or range of extent of polymerization, where such time interval or range of extent of polymerization may be defined with respect to the curve being quantified or with respect to controls. For example, the slope of the curve can be calculated from the time point at which the reaction being quantified or its controls have undergone at least 10% of their total fluorescence change upon polymerization to the time point at which the reaction being quantified or its controls have undergone no greater than 90% of their total fluorescence change upon polymerization, or any other combination of extents of polymerization, such as from at least 0%, 10%, 15%, 20%, 25%, 30%, 35% or 40%, to no greater than 50%, 60%, 65%, 70%, 75%, 80%, 85% or 90% of the total fluorescence change, and so on. The first three of these approaches are graphically illustrated in FIGS. 5A-5F.

The approach taken in certain screening methods includes:

1. Collecting a time series of data from each well in a multi-well plate. As just noted, in the absence of effects from the candidate agent, a sigmoidal curve is typically acquired.

2. Fitting the collected data to a calculated curve if possible using a curve fitting program (e.g., 4 parameter curve fit), including those known in the art, or alternatively, in combination with curve fitting, deriving numerical and slope parameters to characterize the sigmoidal curve.

3. Determining one of more of the four foregoing parameters. The parameters can be determined from a fit to the curve data or from the primary data if the curve fit program does not yield useful data (e.g., a relatively flat signal is obtained overtime indicating strong inhibition).

4. Determining whether the collected data is an accurate indication of polymerization. Incomplete polymerization can be detected for example if there is a smaller increase in fluorescence as compared to other wells on the plate. The presence of an agent that interferes with signal acquisition can be detected if the initial fluorescence intensity is significantly higher or lower than expected, indicating that the candidate agent respectively promotes or inhibits fluorescence by the dye. The presence of high noise levels suggests that the candidate agent has precipitated to form light scattering aggregates or acts as a detergent that forms light-scattering bubbles.

The parameter that is determined for each assay sample is typically compared against one or more controls. The control can be a historic value that was determined prior to the samples currently being investigated for a sample lacking one or more components of the assay or lacking a candidate agent. In other instances, the control is determined contemporaneously with the samples being analyzed. The control can represent a single reading or be a statistical value (e.g., mean or average) determined on the basis of several controls. The comparison process can involve determining whether there is a statistically significant difference between a measured parameter and one obtained for a control.

VIII. Assays for Deconvoluting Mechanism by which Agent Acts

The primary screening assays that are described herein can include a number of different proteins and thus can be utilized to identify agents that target multiple different components of the assay. A series of secondary screening assays can be performed after the primary screening assay to characterize the primary hits and to identify the relevant target protein(s) affected by the candidate agent.

The secondary screening assays in general can utilize any of the components listed above. The general deconvolution strategy involves: 1) confirmation of activity and elimination of false positives; 2) identification of candidate agents that affect actin polymerization directly; 3) identification of candidate agents that act on Arp2/3 directly; and 4) identification of candidate agents acting on the NPF (e.g., WASP) or an upstream regulator (e.g., Cdc42).

One approach for evaluating primary hits is to confirm activity and eliminate false positives by conducting the assay in duplicate or more. Assays can also be conducted with several-fold higher concentrations of Arp2/3, WASP, Cdc42 and PIP₂ (e.g., 2×, 3× or 4×). A typical confirmation assay thus includes: actin (actin and acrylodan-labeled actin or pyrene-labeled actin at a total concentration of 2.5 μM), Arp2/3 (6 nM), FL-WASP (3 nM), Cdc42 (0.5 μM) and optionally PIP₂ (20 μM).

Following confirmation, the effects of compounds on actin polymerization in the absence of an actin nucleator (e.g., Arp2/3), NPF or upstream activator are determined to identify actin interacting compounds, or alternatively using a different actin nucleator (e.g., a formin such as the FH1-FH2 domains of Candida albicans FOR1). In either case, compounds that modulate actin polymerization are highly likely to exert their effects by interacting with actin, since it is the only protein component in common with the primary screening and confirmation assays. In the absence of actin nucleator, polymerization is induced by a high concentration of actin (e.g., 2× the level used in the primary screen). Thus, if the primary screen is conducted at a total actin concentration of 2.5 μM, the secondary screen is conducted at an actin/acrylodan-labeled actin or pyrene-labeled actin concentration of 5.0 μM, where the actin concentration means the total concentration of unlabeled actin and actin labeled with either acrylodan or pyrene.

Confirmed hits that do not affect polymerization by interacting with actin are tested for effects on Arp2/3-stimulated actin polymerization promoted by Listeria monocytogenes ActA activates the Arp2/3 complex and bypasses WASP, Cdc42 and PIP₂, if PIP₂ is present. Positive hits may represent Arp2/3 inhibitors and the others are related to WASP, Cdc42 or PIP₂. An exemplary secondary assay of this type includes: actin (2.5 μM total concentration of unlabeled actin and acrylodan-labeled actin or pyrene-labeled actin), Arp2/3 (20 nM) and Act A (0.25 μM).

Alternatively, confirmed hits that do not not affect polymerization by interacting with actin are tested for effects on Arp2/3-stimulated actin polymerization promoted by a constitutively active form or domain of an NPF, which bypasses the requirement for an upstream activator. Positive hits may represent Arp2/3 or NPF inhibitors and the others are related to Cdc42 or PIP₂. An exemplary secondary assay of this type includes: actin (2.5 μM total concentration of unlabeled actin and acrylodan-labeled actin or pyrene-labeled actin), Arp2/3 (20 nM) and the VCA domain of WASP (3 nM).

Lastly, assays can be performed to assess action on NPF in the assay (e.g., WASP-related protein activation). For WASP, this can be performed using Nck1 instead of Cdc42 to activate WASP. For N-WASP, this can be performed using Shigella IcsA, which activates N-WASP and bypasses Cdc42 and PIP₂. Positive hits, with confirmed activity that do not modulate actin, Arp2/3 or Cdc42, likely modulate the activity of the NPF (e.g., WASP or N-WASP) or its interaction with actin, Arp2/3 or Cdc42. A typical secondary assay of this type includes actin (2.5 μM total concentration of unlabeled actin and acrylodan-labeled actin or pyrene-labeled actin), Arp2/3 (20 nM), FL-WASP (3 nM) and Nck1 (0.5 μM).

IX. Cell-Based Secondary Assays

A series of cell-based secondary assays can be performed on confirmed hits from the in vitro primary and secondary screening assays to identify candidate agents that can affect actin polymerization in vivo. The cell-based secondary assays in general can utilize mammalian cells, which can respond to various stimuli by changes in actin polymerization or can support the directed movement of bacteria in infected host cells. For example, monocyte-derived cells, including macrophage, dendritic and osteclast cells, can form specialized, actin-rich structures, termed podosomes, in response to stimuli (see, e.g., Linder and Aepfelbacher (2003) Trends in Cell Biol. 13:375-385). A variety of mammalian cells support the motility of bacteria, like Listeria monocytogenes, in the cytoplasm of infected host cells (see, e.g., Welch et al. (1997) Nature 385:265-269).

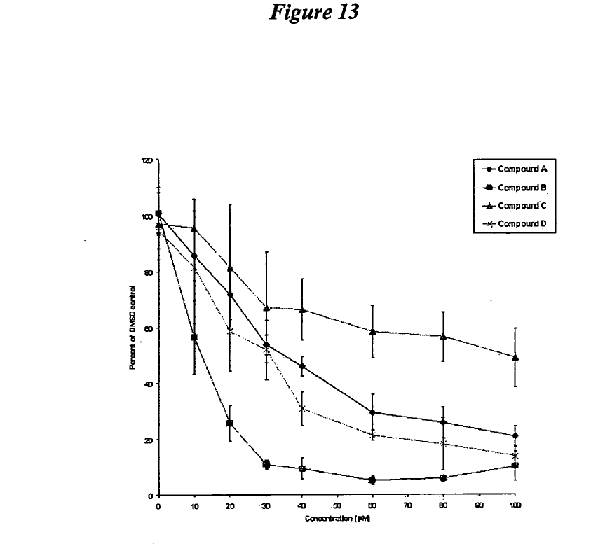

Positive hits from the in vitro screening assays are tested for effects on macrophage podosome formation. Macrophages express Arp2/3 and WASP, the latter of which is required for podosome formation. A typical cell-based secondary assay of this type includes the human monocytic cell ine THP-1, which undergoes macrophage differentiation and podosome formation in response to exposure to the phorbol ester, phorbol-12-myristate-13-acetate (PMA) (50 nM). A nuclear stain and an actin stain are used to detect and quantify the number of podosomes/cell in the absence or presence of positive hits from the in vitro screening assays. An example of the use of this assay is provided in Example 14 and is illustrated in FIG. 13.

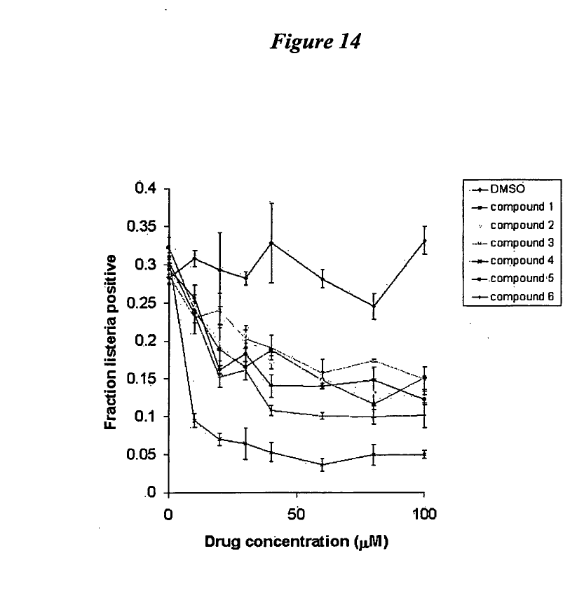

Positive hits from the in vitro screening assays are tested for effects on bacterial motility. Pathogenic bacteria such as Listeria monocytogenes use the host cell actin machinery for motility and spread of infection. The bacterial surface protein ActA directly activates Arp2/3, which is required for actin polymerization and Listeria motility. A typical cell-based secondary assay of this type uses Listeria monocytogenes and SKOV-3 human ovarian cancer cells. An anti-Listeria antibody and an actin stain are used to quantify Listeria motility in a culture of SKOV-3 cells in the absence or presence of positive hits from the in vitro screening assays. An example of the use of this assay is provided in Example 15 and is illustrated in FIG. 14.

X. Exemplary High Throughput Screens

The screening methods that are provided can be conducted in high throughput formats, including the use of robotic systems. High throughput screening (HTS) methods can thus be used to analyze many samples within a short period of time. For example, micro-well plates having 96, 384 and 1536 wells, or as many wells as are commercially available, can be used.

High throughput screening systems are commercially available (see, e.g., Zymark Corp., Hopkinton, Mass.; Air Technical Industries, Mentor, Ohio; Beckman Instruments, Inc., Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems, i.e., Zymark Corp., provide detailed protocols for the various high throughput assays.

In some screening assays, a plurality of assay mixtures are run in parallel with different agent concentrations to obtain a differential response to the various concentrations. One of these concentrations can serve as a negative control, i.e., at zero concentration or below the level of detection. However, in some embodiments, any concentration can be used as the control for comparative purposes.

Some high throughput screening methods that are provided involve providing a library containing a large number of candidate agents potentially having the desired activity. Such “combinatorial chemical libraries” are then screened in one or more assays, as described herein, to identify those library members (e.g., a particular chemical species or subclass) that display a desired characteristic activity. The compounds thus identified can serve as conventional “lead compounds” or can themselves be used as potential or actual therapeutics or agricultural compounds.

For example, in one embodiment, candidate agents are assayed in highly parallel fashion by using multiwell plates. Samples containing single or multiple candidate agents can be placed into each well. Assay components (e.g., the two mixtures described above) can then be added to the samples in each of the wells and the fluorescence of each well on the plate measured by a plate reader. A candidate agent that modulates the function of a component of the assay is identified by an increase or decrease in the level of fluorescence as compared to a control assay in the absence of that candidate agent.

One exemplary HTS system is as follows. The system comprises a microplate input function that has a storage capacity matching a logical “batch” size determined by reagent consumption rates. The input device stores and, delivers on command, barcoded assay plates containing pre-dispensed samples to a barcode reader positioned for convenient and rapid recording of the identifying barcode. The plates are stored in a sequential nested stack for maximizing storage density and capacity. The input device can be adjusted by computer control for varying plate dimensions. Following plate barcode reading, the input device can be adjusted by computer control for varying plate dimensions. Following plate barcode reading, the input device transports the plate into the pipetting device which contains the necessary reagents for the assay. Reagents are delivered to the assay plate with the pipetting device. Tip washing in between different reagents is performed to prevent carryover. A time dependent mixing procedure is performed after each reagent to effect a homogeneous solution of sample and reagents. The sequential addition of the reagents is delayed by an appropriate time to maximize reaction kinetics and readout levels. Immediately following the last reagent addition, a robotic manipulator transfers the assay plate into an optical interrogation device which records one or a series of measurements to yield a result which can be correlated to an activity associated with the assay. The timing of the robotic transfer is optimized by minimizing the delay between “last reagent” delivery and transfer to the optical interrogation device. Following the optical interrogation, the robotic manipulator removes the finished assay plates to a waste area and proceeds to transfer the next plate from pipetting device to optical interrogation device. Overlapping procedures of the input device, pipetting device and optical interrogation device are used to maximize throughput.

In one embodiment, approximately 1,000-2,000 assays are performed per hour and in other instances up to 2,500-3,500 assays are performed per hour.

XI. Candidate Agents

The terms “candidate agent” or “candidate bioactive agent” or “drug candidate” or grammatical equivalents thereof as used herein generally refers to any molecule (e.g., protein, oligopeptide, small organic molecule, polysaccharide, polynucleotide) to be tested in a screening assay.

Candidate agents can be from any of a number of chemical classes, including organic molecules, such as small organic compounds having a molecular weight of more than 100 and less than about 2,500 daltons. Candidate agents can comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines (including derivatives, structural analogs, or combinations thereof), derivatives, structural analogs or combinations thereof.

Candidate agents can be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In an embodiment provided herein, the candidate bioactive agents are proteins. The protein can include naturally occurring amino acids and peptide bonds, or synthetic peptidomimetic structures. For example, homo-phenylalanine, citrulline and noreleucine are considered amino acids for the purposes of the invention. The term “amino acid” also includes imino acid residues such as proline and hydroxyproline. The side chains may be in either the (R) or the (S) configuration. The amino acids can be in the (S) or (L)-configuration. If non-naturally occurring side chains are used, non-amino acid substituents can be used, for example to prevent or retard in vivo degradation.

Candidate agents can also be naturally occurring proteins or fragments of naturally occurring proteins. Thus, for example, cellular extracts containing proteins, or random or directed digests of proteinaceous cellular extracts, can be used. In some instances, the libraries are of bacterial, fungal, viral, and mammalian proteins (e.g., human).

The candidate agents in some instances are peptides of from about 2 to about 30 amino acids, with from about 5 to about 20 amino acids being preferred, and from about 7 to about being particularly preferred. The peptides can be digests of naturally occurring proteins, random peptides, or random peptides. The term “randomized” as used herein means that each nucleic acid or peptide consists of essentially random nucleotides and amino acids, respectively. Since generally these random peptides (or nucleic acids, discussed below) are chemically synthesized, they can incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In some instances, the library is fully randomized, with no sequence preferences or constants at any position. In other instances, the library is biased. That is, some positions within the sequence are either held constant, or are selected from a limited number of possibilities. In some biased libraries, for example, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, and serines, threonines, tyrosines or histidines for phosphorylation sites.

The candidate agents can also be nucleic acids. The nucleic acid includes at least two nucleotides covalently linked together. Nucleic acids generally contain phosphodiester bonds, although in some cases, as outlined below, nucleic acid analogs are included that can have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984); Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, Carbohydrate Modifications in Antisense Research, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carboxylic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These ribose-phosphate backbones can be modified to facilitate the addition of additional moieties such as labels, or to increase the stability and half-life of such molecules in physiological environments.

In addition, mixtures of naturally occurring nucleic acids and analogs can be made. Alternatively, mixtures of different nucleic acid analogs, and mixtures of naturally occurring nucleic acids and analogs may be made. The nucleic acids can be single stranded or double stranded, or contain portions of both double stranded or single stranded sequence. The nucleic acid can be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, etc.

As described above generally for proteins, nucleic acid candidate agents can be naturally occurring nucleic acids, random nucleic acids, or biased random nucleic acids. For example, digests of procaryotic or eukaryotic genomes can be used as is outlined above for proteins.

Still other candidate agents are organic chemical moieties, a wide variety of which are described in the literature and commercially available. Small molecules are one subclass of organic molecules that can be used as candidate agents. The small molecule is usually 4 kilodaltons (kDa) or less. In some instances, the compound is less than 3 kDa, 2 kDa or 1 kDa. In other instances, the compound is less than 800 daltons (Da), 500 Da, 300 Da or 200 Da. In still other instances, the small molecule is about 75 Da to 100 Da, or alternatively, 100 Da to about 200 Da.

Devices for the preparation of combinatorial libraries are commercially available (see, e.g., 357 MPS, 390 NWS, Advanced Chem Tech, Louisville K.Y.; Symphony, Rainin, Woburn, Mass.; 433A Applied Biosystems, Foster City, Calif.; 9050 Plus, Millipore, Bedford, Me.).

It is understood that once a modulator or binding agent is identified that it can be subjected to further assays to further confirm its activity. In particular, the identified agents can be entered into a computer system as lead compounds and compared to others which may have the same activity. The agents may also be subjected to in vitro and preferably in vivo assays to confirm their use in medicine as a therapeutic or diagnostic or in the agricultural

XII. Applications

Because actin polymerization is directly linked to cell motility which in turn is involved in many diseases, agents that modulate components involved in actin polymerization can have use in the treatment of many diseases, or as lead compounds in the development of further optimized compounds for the treatment of disease. Diseases associated with cell motility that may be amenable to treatment with inhibitors include, for example, autoimmune diseases, inflammatory diseases, metastatic cancers, and conditions associated with hyperactivity of platelets or increased risk of blood clotting. Inhibitory agents can also be utilized to at least partially recapitulate Wiskott-Aldrich Syndrome, thus making the inhibitors useful in studying this disease.

WASP is expressed in high levels primarily in hematopoietic cells and is the main NPF species represented in these cells. This means that inhibitors of WASP can be useful in selectively targeting immune diseases and inflammation. Specific examples include, but are not limited to, eczema, hemolytic anemia, vasculitis, renal disease, transient and chronic arthritis, formation of lesions in blood vessel walls in heart disease, multiple sclerosis, lupus erythematosis, Crohn's disease, and thrombus formation and secretion of inflammatory and pro-thrombotic cytokines by platelets. Inhibitors can also be used in immune suppression (e.g., prevention of organ tansplant). WASP is present in hematopoietic cells such as those that mediate inflammatory processes and produce platelets, and mediates both signal transduction events and actin dynamics involved in cell motility. Therefore, use of inhibitors identified by the screening methods can be utilized to prevent chemotaxis of immune cells to sites of inflammation or their activation once present.

WAVE1 and WAVE3 are overexpressed in brain tissue, indicating that inhibitors of these two proteins may be useful in treating various neurological disorders (e.g., Alzheimer's, epilepsy, and stroke). WAVE3 expression is generally down regulated in tumor samples, indicating that some active agents identified by the screening methods can be used in tumor treatment.

The modulators identified herein can be utilized to inhibit a variety of types of cell motility. Examples of the types of cellular motility the actin nucleators, NPC and upstream activators are involved in are summarized in Table 1. The types of motility include laemillipodia, filopodia, phagocytosis, endocytosis movement of pathogens. Types of cellular activities these various proteins are involved are also listed. So if the goal is to target a particular type of cellular motility or a particular cellular activity, Table 1 provides a guide for the type of proteins that should be included in the assay. In this way, one can identify modulators that have some specificity for the particular type of cell motility or activity of interest.

Active agents identified by the screening methods described herein can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al. (1989) Computer-Assisted Modeling of Receptor-Ligand Interactions (Alan Liss, New York).

Once analogs have been prepared, they can be screened using the methods disclosed herein to identify those analogs that exhibit an increased ability to modulate the activity of a specific component of actin polymerization. Such compounds can then be subjected to further analysis to identify those compounds that appear to have the greatest potential as pharmaceutical agents. Alternatively, analogs shown to have activity through the screening methods can serve as lead compounds in the preparation of still further analogs, which can be screened by the methods described herein. The cycle of screening, synthesizing analogs and rescreening can be repeated multiple times.

Agents identified by the screening methods described above and analogs thereof can serve as the active ingredient in pharmaceutical compositions formulated for the treatment of various diseases such as those just listed. Active agents identified by the screening methods, can be formulated for use as pharmaceutical compositions. Such compositions can also include, for example, depending on the formulation desired, pharmaceutically acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

XIII. Kits

Kits for conducting the assays or screening methods that are disclosed herein are also provided. The kits in general include the assay components necessary to conduct an actin polymerization assay. The components making up the kits are typically stored in individual containers or combined in a single container, provided the agents that are combined are not reactive with one another.

Some kits for assaying for actin polymerization, for instance, include one, some or typically all of the following: purified actin; acrylodan-labeled-G-actin or pyrene-labeled-G-actin, a purified actin nucleator such as those described herein; a purified NPF protein such as those described herein; and a purified upstream regulator such as those described herein. Some kits can include only the actin components needed for polymerization, e.g., purified actin and either acrylodan-labeled-G-actin or pyrene-labeled-G-actin. The kits can also include other components such as a buffer, an antifoaming agent or surfactant, a mixture of polymerization salts (e.g., a mixture of MgCl₂ and KCl), and a multiwell assay plate. Typically, instructions for using the kit components to perform the assay and screening methods disclosed herein are also included in the kits.

The following examples are offered to illustrate certain aspects of the methods that are described herein and thus should not be construed to limit the claimed invention.

### EXAMPLE 1

### Purification of the Arp2/3 Complex

This example provides a description of an exemplary method for preparing purified Arp2/3 that can be utilized in the polymerization assays disclosed herein.

A. Materials

1. Buffer A:
10 mM Tris pH 8.0 (room temperature), 1 mM DTT, 1 mM MgCl, 30 mM KCl, 0.2 mM ATP, 1 mM EGTA KOH (0.25M stock pH 7) and 2% Glycerol.

2. DEAE Buffer:
Buffer A plus 2 tablets of protease inhibitors/L and 1 mM PMSF.

3. Lysis Buffer:
50 mM Tris; 50 mM KCl; 10 mM Imidazole; 1 mM DTT; pH 7.0.

4. Tris Wash Buffer:
50 mM Tris; 50 mM KCl; 25 mM Imidazole, 1 mM DTT; pH 7.0.

5. Elution Buffer:
50 mM Tris; 300 mM Imidazole; 50 mM KCl; 1 mM DTT; pH 7.4.

6. DEAE Chromatography Material (TOYOPEARL DEAE-650M; product #07473; manufactured by Tosh).

7. Q Sepharose Chromatography Material (Q Sepharose Fast Flow; product #17-0510-01, from Amersham Biosciences)

B. Preparation of Affinity Column Matrix

1. Synthesis and Expression of GST-VCA-His Fusion
WASP full length cDNA is used as a template to amplify the coding sequence.
   Oligo (forward): 5′-GGGGACAAGTTTGTACAAAAAAGCAGGCTTCGAAAACCTGTATTTTCAGGGCGG GGGTCGGGGAGCGCTTTTGGATC-3′ (SEQ ID NO:49) and oligo (reverse): 5′-GGGGACCACTTTGTACAAGAAAGCTGGGTCCTAGTGATGGTGATGGTGATGGTA GTACGAGTCATCCCATTCATCATCTTCATC-3′ (SEQ ID NO:50) are used in the reaction.
The pcr fragment is cloned into pDONR201 (Invitrogen Life Technology, Cat# 11798-014) by Gateway BP reaction to generate pDONR_tev_WASPVCA_His.
Clone pDONR_tev_WASPVCA_His into pDEST15 (Invitrogen Life Technology, Cat# 11802-014) to generate N-GST_tev_WASPVCA_His by LR Gateway recombination reaction.
The cloned DNA can be expressed as described in Example 5.

2. Purification of GST-VCA-His Fusion Protein
a. Growth conditions:
   Inoculate culture in the morning with a single fresh colony (use B121 (DE3)lysP cells). Use LB medium with (i.e., Sigma T-9179 or Gibco/BRL 22711-022) with 10 ppm antifoam.
   Typical volume for a prep is 1-2 L. Use white baffled flask for 1 L of culture.
   Grow at 37° C. with shaking until OD₆₀₀ reaches 1.0-1.2.
   Shake at RT for 30-45 min.
   Add IPTG to 0.5 mM; continue shaking O/N.
b. Harvest cells following morning (after 12-16 hours) by spinning in a bench top Beckman centrifuge at 3 Krpm or in a JLA 10 rotor at 5 Krpm for 30 minutes at 4° C.
From this point keep solutions on ice and/or at 4° C.
c. Resuspend cell pellets in Lysis buffer supplemented with 1× concentrations of Complete EDTA-free protease inhibitors (Boehringer 1836 170; use 1 mini-tablet per 10 ml) (20 ml for 1 L culture, 40 ml for 2 L). Use dounce homogenizer to make sure resuspension is complete. Proceed with a preparation or freeze cell suspension in liquid N₂ and store at −80° C.
d. Cell disruption: When thawing cells add BME fresh. Lyze cells with the Microfluidizer by running 2 passes, 7-8 cycles each at 80 psi (on the green scale). (If using frozen cells, do 1 pass of 3 cycles). Pass some extra buffer (˜10 ml) through the chamber to rinse it.
e. Spin lysate in 45Ti rotor at 35 Krpm at 4° C. for 30 min. During this spin pre-equilibrate the resin with lysis buffer (see below).
f. Pre-equilibrate 1.5-2 ml (for 1 L culture) or 3 ml (for 2 L culture) of Ni-NTA resin (Qiagen Cat# 31014) with Lysis buffer by washing 2 times with 15 ml of buffer without DTT and protease inhibitors. During these washes collect resin by spinning at 600-700 rpm for 2 min in a bench-top centrifuge.
g. Collect supernatant (save a sample for a gel). Batch load it onto Ni-resin. Incubate at 4° C. for 1 hr with rocking.
h. Pellet the resin by spinning at 600-700 rpm for 2 min. Decant supernatant (save sample for a gel). Resuspend in 5-10 ml of Lysis buffer (with BME and ˜ 1/10 of Complete inhibitors—i.e. 1 mini-tablet per 100 ml) and load resin into a column (use disposable columns or BioRad 1 cm ID EconoColumns). Wash with 50 ml of Lysis buffer. Washes can be done by gravity flow or with a peristaltic pump at 1 ml/min.
i. Pass 10 ml of Tris Wash Buffer through the column.
j. Elute with 8 1 ml fractions with Elution Buffer with 1/10 of protease inhibitors. Check protein concentrations in fractions by Coomassie Plus (Bradford). Pool peak fractions (protein usually elutes starting at fraction 3).
Measure protein concentration in pooled fractions. Dilute with Tris Wash Buffer + 1/10 protease inhibitors to 2 mg/ml.
k. Freeze in liquid N₂ by “drop-freezing”. Store at −80° C.

3. Forming Affinity Matrix
The purified GST-VCA-His fusion is coupled to Glutathione-Sepharose (Amersham Biosciences) or related material according to the manufacturer's instructions.

C. Purification of Arp2/3

1. A cellular extract containing Arp2/3 complex was prepared from an Arp2/3 source such as human platelets (see, e.g., U.S. Provisional Application No. 60/578,969, filed Jun. 10, 2004; Welch and Mitchison (Meth. Enzymology 298:52-61,1988); and Higgs, H. N. et al. (Biochemistry 38:15212-15222, 1999), all of which are incorporated herein by reference in their entirety for all purposes).

2. A DEAE column was packed with DEAE material and equilibrated with DEAE buffer. The amount of DEAE material included in the column was calculated based on 250 ml of resin for each 100 ml of crude extract.

3. The conductivity of the extract was adjusted to approximately 30 mM salt (3.6 mS is equivalent to 30 mM salt) and then loaded onto the DEAE column. Flowthrough was collected and the DEAE column washed with about 2 column volumes of DEAE buffer, which was also collected.

4. A Q-Sepharose column was packed and equilibrated with Buffer A. The amount of material was calculated based upon 100 ml of column material for each 200 ml of extract). The collected flowthrough and wash solution was loaded onto the equilibrated column. The column was then washed with 5-10 column volumes of Buffer A containing 30 mM KCl to displace proteins that did not bind or only loosely bound the column material. Bound proteins, including Arp2/3 complex, were subsequently eluted in Buffer A with a salt gradient of 30-300 mM KCl.

5. Fractions containing Arp2/3 were identified using the assay methods described herein and active fractions collected. The pooled fractions were diluted to obtain a conductivity of about 3.6 mS.

6. An affinity chromatography column containing the affinity matrix described above (i.e., GST-VCA-His6) was equilibrated in Buffer A. Pooled fractions enriched in Arp2/3 complex were then loaded onto the affinity column. The column was washed with about 5 volumes of Buffer A containing 30 mM KCl. Arp2/3 complex was eluted from the affinity column with 250 mM KCl in Buffer A.

7. Eluted fractions from the affinity column containing purified Arp2/3 were identified. Active fractions were concentrated in Y30 Centricons. The purified Arp2/3 was then diluted with fresh Buffer A to obtain a final solution containing about 30 mM KCl. Glycerol was added to about 30% (v/v) and the final protein solution stored at −20° C. The final protein had a purity of about 95% or more.

### EXAMPLE 2

### Cloning of WASP Proteins

A. Cloning of WASP VCA Domain
1. WASP full length cDNA is used as a template to amplify the coding sequence. Oligo (forward): 5′-GGGGACAAGTTTGTACAAAAAAGCAGGCTTCGAAAACCTGTATTTTCAGG GCGGGGGTCGGGGAGCGCTTTTGGATC-3′ (SEQ ID NO:49) and oligo (reverse): 5′-GGGGACCACTTTGTACAAGAAAGCTGGGTCCTAGTCATCCCATTCATCATC TTCATC-3′ (SEQ ID NO:51) are used in the reaction.
2. The pcr fragment is cloned into pDONR201 (Invitrogen Life Technology, Cat# 11798-014) by Gateway BP reaction to generate pDONR_tev_HsWASPVCA.
3. Clone pDONR_tev_HsWASPVCA into pDEST15 (Invitrogen Life Technology, Cat# 11802-014) to generate N-GST_tev_HsWASPVCA by LR Gateway recombination reaction.

B. Cloning of N_GST_{—}105WASP (bacterial GST tagged protein)
1. WASP full length is used as a template to amplify the coding sequence. Oligo (forward): 5′-CACCGAAAACCTGTATTTTCAGGGCCTTGTCTACTCCACCCCCACCCCC-3′ (SEQ ID NO:52) and oligo (reverse): 5′-CTAGTCATCCCATTCATCATCTTC-3′ (SEQ ID NO:53) are used in the reaction.
2. The pcr fragment is cloned into pENTR/SD/TOPO vector (Invitrogen Life Technology, Cat# K2400-20) by directional cloning using Topoisomerase 1.
3. The pENTR/SD/TOPO_{—}105LWASP is cloned into pDEST15 (Invitrogen Life Technology, Cat# 11802-014) by Gateway LR reaction to generate N_GST_{—}105LWASP.

C. Cloning of pcDNA3.1Myc_{—}105LWASPTAP (Mammalian TAPTAG Tagged Protein)
1. WASP full length (American Type Culture Collection, Cat# 99534) is used as a template to amplify the coding sequence. Oligo (forward): 5′-GGGGACAAGTTTGTACAAAAAAGCAGGCTTCCTTGTCTACTCCACCCCCA CCCCC-3′ (SEQ ID NO:54) and oligo (reverse): 5′-GGGGACCACTTTGTACAAGAAAGCTGGGTCGTCATCCCATTCATCATCTTC ATC-3′ (SEQ ID NO:55) are used in the reaction.
2. The pcr fragment is cloned into pDONR201 vector (Invitrogen Life Technology, Cat# 11798-014) by Gateway BP reaction to generate pDONR WASP 105L.
3. The pDONR WASP 105L is cloned into pcDNA3.1MycTAP vector converted to Gateway destination vector by insertion a Gateway reading frame cassette.

D. Cloning of pcDNA3.1Myc_WASPTAP (Mammalian TAPTAG Tagged Protein)
1. WASP full length (American Type Culture Collection, Cat# 99534) is used as a template to amplify the coding sequence. Oligo (forward): 5′-GGGGACAAGTTTGTACAAAAAAGCAGGCTTCATGAGTGGGGGCCCAATG GGAGG-3′ (SEQ ID NO:56) and oligo (reverse): 5′-GGGGACCACTTTGTACAAGAAAGCTGGGTCGTCATCCCATTCATCATCTTC ATC-3′ (SEQ ID NO:55) are used in the reaction.
2. The pcr fragment is cloned into pDONR201 vector (Invitrogen Life Technology, Cat# 11798-014) by Gateway BP reaction to generate pDONR WASP fl.
3. The pDONR WASP fl is cloned into pcDNA3.1 MycTAP vector converted to Gateway destination vector by inserting a Gateway reading frame cassette by Gateway LR reaction.

### EXAMPLE 3

### Cloning of N-WASP Proteins

A. Cloning of GST_N-WASPVCA
1. N-WASP full length cDNA is used as a template to amplify the coding sequence. Oligo (forward): 5′-GGGGACAAGTTTGTACAAAAAAGCAGGCTTCGAAAACCTGTATTTTCAGG GCTCTGATGGGGACCATCAG-3′ (SEQ ID NO:57) and oligo (reverse): 5′-GGGGACCACTTTGTACAAGAAAGCTGGGTCCTAGTCTTCCCACTCATCAT CATCCTC-3′ (SEQ ID NO:58) are used in the reaction.
2. The pcr fragment is cloned into pDONR201 (Invitrogen Life Technology, Cat# 11798-014) by Gateway BP reaction to generate pDONR_tev_HsNWASPVCA.
3. Clone pDONR_tev_HsN-WASPVCA into pDEST15 (Invitrogen Life Technology, Cat# 11802-014) to generate N-GST_tev_HsN-WASPVCA by LR Gateway recombination reaction.

B. Cloning of N_GST_tev_{—}98FN-WASP (Bacterial GST Tagged Protein)
1. pENTR/SD/TOPO_N-WASP full length is used as a template to amplify the coding sequence. The oligo (forward): 5′-CACCGAAAACCTGTATTTTCAGGGCTTTGTATATAATAGTCCTAGAGGATA TTTTC-3′ (SEQ ID NO:59) and oligo (reverse): 5′-TTAGTCTTCCCACTCATCATCATC-3′ (SEQ ID NO:60) are used in the reaction.
2. The pcr fragment is cloned into /SD/TOPO vector (Invitrogen Life Technology, Cat# K2400-20) by directional cloning using Topoisomerase 1.
3. The pENTR/SD/TOPO_tev_{—}98FN-WASP is cloned into pDEST15 (Invitrogen Life Technology, Cat# 11802-014) by Gateway LR reaction to generate N_GST_tev_{—}98FN-WASP.

C. Cloning of pcDNA3.1Myc_{—}98FN-WASPTAP (mammalian TAPTAG tagged protein)

1. The pENTR/SD/TOPO_tev_{—}98FN-WASP is used as a template to amplify the coding sequence. Oligo (forward): 5′

| | |
|---|---|
| Oligo (forward): | |
| 5′-GGGGACAAGTTTGTACAAAAAAGCAGGCTTCGA | (SEQ ID NO:61) |
| AAACCTGTATTTTCAGGGCTTTGTATATAATAGTCC | |
| TAGAGG-3′ | |
| and | |
| oligo (reverse): | |
| 5′-GGGGACCACTTTGTACAAGAAAGCTGGGTCGTC | (SEQ ID NO:62) |
| TTCCCACTCATCATCATCCTC-3′. | |

2. The pcr fragment is cloned into pDONR201 vector (Invitrogen Life Technology, Cat# 11798-014) by Gateway BP reaction to generate pDONR 98FN-WASP.
3. The pDONR 98FN-WASP is cloned into pcDNA3.1MycTAP vector converted to Gateway destination vector by insert a Gateway reading frame cassette by Gateway LR reaction.

D. Cloning of pcDNA3.1Myc_N-WASPTAP (Mammalian TAPTAG Tagged Protein)
1. HeLa total RNA is used as a template to amplify N-WASP by using SuperScript II RNase H-Reverse Transcriptase (Invitrogen Life Technology, Cat#18064-014). The oligo (forward): 5′-CACCGAAAACCTGTATTTTCAGGGCAGCTCCGTCCAGCAGCAGCCGCCG-3′ (SEQ ID NO:63) and oligo (reverse): 5′-TCAGTCTTCCCACTCATCATCATC-3′ (SEQ ID NO:64) are used in the reaction.
2. The pcr fragment is cloned into pENTR/SD/TOPO vector (Invitrogen Life Technology, Cat# K2400-20) by directional cloning using Topoisomerase 1.
3. pENTR_N-WASP/SD/TOPO is used as a template to amplify the coding sequence. Oligo (forward): 5′-GCCGCTCGAGGTCTTCCCACTCATCATCATC-3′ (SEQ ID NO:65) and oligo (reverse): 5′-GCCGCTCGAGATGAGCTCCGTCCAGCAGC-3′ (SEQ ID NO:66) are used in the reaction.
4. The pcr fragment is digested with XhoI endonuclease and ligated into calf intestinal alkaline phosphatase (CIAP) treated pcDNA3.1MycTAP vector.
5. Orientation of insert is checked to generate pcDNA3.1Myc_N-WASPTAP.

### EXAMPLE 4

### Cloning of Upstream Regulatory Proteins

A. Cloning of N_GST_tev_Cdc42 GTP (Bacterial GST Tagged Cdc42 Protein)
1. pDONR_tev_Cdc42 wt is used as a template for QuickChange site-directed mutagenesis (Stratagene, Cat# 200518). Oligo (forward): 5′-TGTGTTGTTGTGGGCGATGTTGCTGTTGGTAAAACATGT-3′ (SEQ ID NO:67) and oligo (reverse): 5′-ACATGTTTTACCAACAGCAACATCGCCCACAACAACACA (SEQ ID NO:68) are used in this reaction to mutate G12 to a V.
2. Clone pDONR_tev_Cdc42GTP into pDEST15 (Invitrogen Life Technology, Cat# 11802-014) to generate N-GST-tev-Cdc42GTP by LR Gateway recombination reaction.

B. Cloning of N_GST_tev_RhoC GTP (Bacterial GST Tagged RhoC Protein)
1. pDONR_tev_RhoC wt is used as a template for QuickChange site-directed mutagenesis (Stratagene, Cat# 200518). Oligo (forward): 5′-GTGATCGTTGGGGATGTTGCCTGTGGGAAGGAC-3′ (SEQ ID NO:69) and oligo (reverse): 5′-GTCCTTCCCACAGGCAACATCCCCAACGATCAC (SEQ ID NO:70) are used in this reaction to mutate G14 to a V.
2. Clone pDONR_tev_RhoC GTP into pDEST15 (Invitrogen Life Technology, Cat# 11802-014) to generate N-GST_tev_RhoC GTP by LR Gateway recombination reaction. An exemplary encoding sequence is SEQ ID NO:27.

C. Cloning of N_GST_tev_RhoA GTP (Bacterial GST Tagged RhoA Protein)
1. RhoA GTP is used as a template to amplify the RhoA GTP coding sequence. Oligo (forward): 5′-GGGGACAAGTTTGTACAAAAAAGCAGGCTTCGAAAACCTGTATTTTCAGG GCGCTGCCATCCGGAAGAAACTGGTG-3′ (SEQ ID NO:71) and oligo (reverse): 5′-GGGGACCACTTTGTACAAGAAAGCTGGGTCCTACAAGACAAGGCAACCAC ATTTTTTC-3′ (SEQ ID NO:72) are used in this reaction.
2. Clone pcr fragment into pDONR201 vector (Invitrogen Life Technology, Cat# 11798-014) by Gateway BP reaction to generate pDONR_tev_RhoA GTP.
3. Clone pDONR_tev_RhoAGTP into pDEST15 (Invitrogen Life Technology, Cat# 11802-014) to generate N-GST_tev_RhoA GTP by LR Gateway recombination reaction. An exemplary encoding sequence is SEQ ID NO:29.

D. Cloning of N_GST_tev_Rac1 GTP (Bacterial GST Tagged Rac1 Protein)
1. Rac1 GTP is used as a template to amplify the Rac1 GTP coding sequence. Oligo (forward): 5′-GGGGACAAGTTTGTACAAAAAAACGGGCTTCGAAAACCTGTATTTTCAGG GCCAGGCCATCAAGTGTGTGGTGGTG-3′ (SEQ ID NO:73) and oligo (reverse): GGGGACCACTTTGTACAAGAAAGCTGGGTCCTACAACAGCAGGCATTTTC TCTTCCTC-3′ (SEQ ID NO:74) are used in this reaction.
2. Clone pcr fragment into pDONR201 vector (Invitrogen Life Technology, Cat# 11798-014) by Gateway BP reaction to generate pDONR_tev_Rac1 GTP.
3. Clone pDONR_tev_Rac1 GTP into pDEST15 (Invitrogen Life Technology, Cat# 11802-014) to generate N-GST_tev_Rac1 GTP by LR Gateway recombination reaction. An exemplary coding sequence is SEQ ID NO:31.

E. Cloning of N_GST_tev_Nck1 (Bacterial GST Tagged Nck1 Protein)
1. Nck cDNA (American Type Culture Collection, Cat# MGC-12668/4304621) is used as a template to amplify the Nck1 coding sequence. Oligo (forward): 5′-GGGGACAAGTTTGTACAAAAAAGCAGGCTTCGAAAACCTGTATTTTCAGG GCATGGCAGAAGAAGTGGTGGTAGTAG-3′ (SEQ ID NO:75) and oligo (reverse): 5′-GGGGACCACTTTGTACAAGAAAGCTGGGTCCTATGATAAATGCTTGACAA GATATAA-3′ (SEQ ID NO:76) are used in the reaction.
2. Clone pcr fragment into pDONR201 vector (Invitrogen Life Technology, Cat# 11798-014) by Gateway BP reaction to generate pDONR_tev_Nck1 GTP.
3. Clone pDONR_tev_Nck1 into pDEST15 (Invitrogen Life Technology, Cat# 11802-014) to generate N-GST_tev_Nck1 by LR Gateway recombination reaction. An exemplary coding sequence is SEQ ID NO:33.

F. Cloning of GST_NCK2
1. NCK2 full length cDNA is used as a template to amplify the coding sequence. Oligo (forward): 5′-CACCATGACAGAAGAAGTTATTGTGATAGCC-3′ (SEQ ID NO:77) and oligo (reverse): 5′-TCACTGCAGGGCCCTGACGAGGTAGAG-3′ (SEQ ID NO:78) are used in the reaction.
2. The pcr fragment is cloned into pENTR/SD/TOPO vector (Invitrogen Life Technology, Cat# K2400-20) by directional cloning using Topoisomerase I.
3. The pENTR/SD/TOPO_NCK2 is cloned into pDEST15 (Invitrogen Life Technology, Cat# 11802-014) by Gateway LR reaction to generate N_GST_NCK2. An exemplary coding sequence is SEQ ID NO:45.

### EXAMPLE 5

### Bacterial Expression of Fusion Proteins

Transformation: Competent cells (BL21(DE3) or BL21 STAR; Invitrogen) are thawed on ice and approximately 1 μl of DNA is added. Cells are gently mixed and incubated on ice for approximately 30 minutes. After heat shock at 42° C. for 45 seconds, cells are incubated on ice for 2 minutes and 0.5 ml SOC medium is added. Cells are allowed to recover by shaking at 37° C. for one hour, and then plated on selective media (typically LB+100 μg/ml ampicillin).

Day 1
For each new stock test for protein expression:
   1. Inoculate several (2-4) 5-10 ml LB-Amp (75 μg/ml Ampicillin) cultures with small fractions of colonies. Mark colonies on a plate to be able to identify mother colony for each culture. Store plate at 4° C. Grow inoculated cultures at 37° C. with shaking until OD₆₀₀=0.8-1. Remove 500 μl sample and collect cells by spinning the sample in an Eppendorf centrifuge 14 Krpm for 2 min; resuspend pellets in 100 μl SDS sample buffer.
   2. Add IPTG to 0.5 mM to the remaining culture. Continue growing at 37° C. for 4 hours or at RT overnight.
   3. Take another set of 500 μl gel samples: collect cells by spinning on an Eppendorf centrifuge 14 Krpm for 2 min; resuspend pellets in 100 μl SDS sample buffer; load 5 μl of each sample on a gel.

Day 2 (or 3)
1. Inoculate 250-500 ml of LB-Amp medum with a single tested colony.
2. Grow at 37° C. with shaking to OD₆₀₀˜0.6-0.8.
3. Collect cells by centrifugation on a table top centrifuge at 3 Krpm for 30 mm.
4. Resuspend in 1/10 of initial volume in cold fresh LB-Amp/10% DMSO. Keep cell suspension on ice.
5. Pipette in 1 ml aliquotes.
6. Freeze in LN₂. Store at −80° C.

### EXAMPLE 6

### Expression and Purification of Full Length WASP

TAPTAG WASP DNA is transfected using the Freestyle™ 293 expression system (Invitrogen Life Technologies, Cat# K9000-01) in a scaled-up protocol:

A. Preparation of Cells for Transfection

(1) Freestyle™ 293-F cells are cultured in Freestyle™ culture medium according to manufacturers' directions (8% CO₂, 37° C.)
(2) Cells are split at 3×10⁵ cells/ml into 5×1000 ml sterile disposable PETG shaker flasks (Nalge Nunc Int, 4112-1000) with 0.45 μm vented closures (Nalge Nunc Int, 4114-0045) at 400 ml per flask and cultured on a shaking platform at 125 rpm for 96 hrs
(3) Cells are then expanded to 10×1000 ml shaker flasks (400 ml/flask) at 1.1×10⁶ cells/ml

B. Transfection of Cells
(1) Add 5.2 ml of 293fectin™ to 140 ml of room temperature Opti-MEM® I reduced serum medium (Invitrogen Life Technologies, Cat 31985-070). Incubate at RT for 5 minutes
(2) Meanwhile, add 4 mg of pcDNA3.1_myc_TAP_WASP (prepared by QIAGEN Plasmid Giga Kit, Cat 12191) to 140 ml of room temperature Opti-MEM® I reduced serum medium
(3) Add the diluted DNA solution to the diluted 293fectin™ solution and incubate at RT for 20 minutes
(4) Add 28 ml of this DNA/lipid mixture to each flask and then culture cells on a shaking platform at 125 rpm for 48 hrs

C. Preparation of Cells for TAPTAG WASP Purification
(1) Pool all flasks (to 4 liters total volume) and count cells
(2) Spin down cells (at 1500 rpm, 8 minutes, 4° C.) and resuspended in 1/10 volume (400 ml) ice cold PBS. Spin again (at 1500 rpm, 8 minutes, 4° C.) and freeze down cells in aliquots of 2.4×10⁹ cells in 50 ml sterile tubes using liquid nitrogen.

D. Purification of TAPTAG WASP

Cool down 500 ml of H₂O

RIPA FOR TAP-TAG STOCK 2×:

```
  10 mM TRIS pH 8.0
  2 mM EDTA
  2 mM EGTA
20% Glycerol
300 mM NaCl
```

Make 500 ml of the 2× buffer, filter and leave on 4° C.

To make 1× RIPA buffer just before using add:

| Final | | Stock | For 10 ml | | For 20 ml | |
|---|---|---|---|---|---|---|
| 1X | Stock | 2X | 5 | ml | 10 | ml |
| 1% | NP-40 | 20% | 500 | μl | 1 | ml |
| 0.125% | Deoxycholate | 5% | 0.5 | ml | 1 | ml |
| 1 mM | PMSF | 1M | 10 | μl | 20 | μl |
| | Inhibitors tablet | | 1 (small) | | 2 (small) | |
| 1 mM | Na₃VO₄ | 0.2 M | 50 | μl | 100 | μl |
| 1 mM | NaF | 0.5M | 20 | μl | 40 | μl |
| 20 mM | Beta glycerophosphate | | | | | |
| | H₂0 | | To 20 | ml | To 20 | ml |

To lyse cells: cover them with 1 ml of ice cold RIPA 1× buffer. Incubate them for 5 min, scrape them and leave for additional 25 min. Scrape again and transfer to cold 3 ml centrifuging tubes (Beckman). Wash plates with 0.2 ml RIPA 1× buffer and transfer solutions to the tubes. Spin for 66 Krpm 10 min (with 100 Krpm temperature rises).

Wash 400 μl (total) of IgG-Sepharose (Pharmacia) 4 times (4×10 ml) with IPP150.

IPP150:

| Final | | Stock | For 100 mL | |
|---|---|---|---|---|
| 10 mM | Tris-Cl pH8.0 | 1M stock | 1 | mL |
| 150 mM | NaCl | 5M | 3 | mL |
| 0.1% | NP40 | 20% | 0.5 | mL |
| | H₂0 | | To 100 | mL |

Pour cell lysate into 15 ml BIO-RAD column and add IgG resin. Shake for 2 h in cold room. Remove the top plug first, then the bottom plug and allow the column to drain by gravity flow.
Wash with 30 mL IPP150.
Wash with 10 mL TEV cleavage buffer.

TEV Cleavage Buffer:

| Final | | Stock | For 100 ml | |
|---|---|---|---|---|
| 10 mM | Tris-Cl pH8.0 | 1 M | 1 | mL |
| 150 mM | NaCl | 5 M | 3 | mL |
| 0.1% | NP40 | 20% | 0.5 | mL |
| 0.5 mM | EDTA | 0.5 M | 100 | μl |
| 1 mM | DTT | 1 M | 100 | μl |
| | H₂0 | | To 100 | mL |

Close the bottom of the column and add 1 ml of TEV buffer with 3 μl of TEV protease (19 mg/ml). Shake for 1 h at RT.

Meanwhile, wash 200 μl of Calmodulin resin (Upstate) with CBB (Calmodulin binding buffer).

CBB—Calmodulin Binding Buffer:

| Final | | Stock | For 100 mL | |
|---|---|---|---|---|
| 10 mM | Tris-Cl pH8.0 | 1 M | 1 | mL |
| 150 mM | NaCl | 5 M | 3 | mL |
| 0.1% | NP40 | 20% | 0.5 | mL |
| 1 mM | MgCl₂ | 1 M | 100 | μl |
| 10 mM | BME (2- | 14.3 M | 69.9 | μl |
| | mercaptoethanol) | | | |
| 1 mM | Imidazole | 0.5 M | 200 | μl |
| 2 mM | CaCl₂ | 1 M | 200 | μl |
| | H₂0 | | To 100 | mL |

Remove the top and bottom plugs of the column and recover the eluate into the new 5 ml column by gravity flow. Elute the solution remaining in old column with an additional 300 μL of TEV cleavage buffer.

To the previous 1 mL eluate add:
3 volumes of calmodulin binding buffer (3 mL) and
3 μL CaCl₂ 1 M per mL of IgG eluate to titrate the EDTA coming from the TEV cleavage buffer.

After closing the column, rotate for 1 hour at 4° C. After binding, allow the column to drain by gravity flow.
Wash with 30 mL CBB.

Elute 10 fractions of 100 μl with CEB calmodulin elution buffer. To elute add elution buffer ⅓ of the column volume, let the flow through come out. Close the column and incubate for 30 min. No shaking is required. Elute 10 100 μl fractions into siliconized tubes.

CEB-Calmodulin Elution Buffer:

| Final | | Stock | For 10 ml | |
|---|---|---|---|---|
| 10 mM | Tris-Cl pH8.0 | 1 M | 0.1 | mL |
| 150 mM | NaCl | 5 M | 0.3 | mL |
| 0.1% | NP40 | 20% | 50 | μl |
| 1 mM | MgCl₂ | 1 M | 10 | μl |
| 10 mM | BME (2- | 14.3 M | 7 | μl |
| | mercaptoethanol) | | | |
| 1 mM | Imidazole | 0.5 M | 20 | μl |
| 20 mM | EGTA | 0.25 M | 800 | μl |
| | H₂0 | | To 10 | mL |

Analogous procedures were utilized with TAPTAG N-WASP DNA, prepared as described in Example 3, to express and purify full length N-WASP.

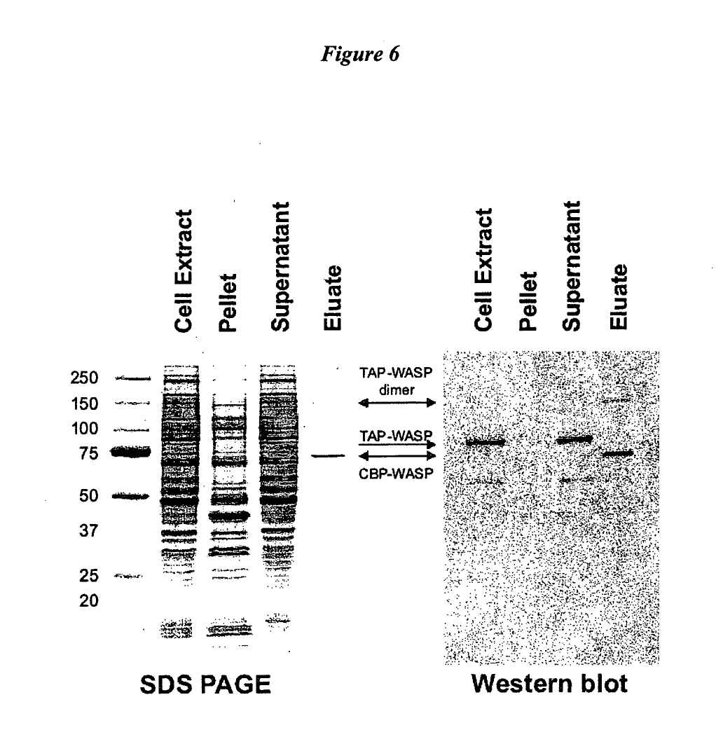

Full-length WASP or N-WASP produced according to the foregoing methods was at least 95% pure and was completely soluble. As shown in FIG. 6, no protein but WASP was observed in purified fractions.

### EXAMPLE 7

### Actin Polymerization Assay Protocol

A. Materials

G-Actin: Typically chicken actin was used. G-actin can be purchased from Cytoskeleton, Inc. It can also be purified according to Pardee and Spudich (1982) Methods of Cell Biol. 24:271-89, and subsequently gel filtered as discussed by MacLean-Fletcher and Pollard (1980) Biochem Biophys. Res. Commun. 96:18-27.

Acrylodan-Actin and Pyrene-Actin: Typically chicken actin was utilized. Pyrene labeled actin was prepared according to methods described in Kouyama and Mihashi (1981) Eur. J. Biochem. 114:33-38 or as described by Cooper et al. (1983) J. Muscle Res. Cell Motility 4:253-62. Alternatively, it can be purchased from Cytoskeleton, Inc. Acrylodan-labeled actin was prepared by modification of the pyrene-labeling protocol described in Cooper et al. (1983) J. Muscle Res. Cell Motility 4:253-62.

GST-Cdc42: Prepared as described in Examples 4 and 5.

GST-105WASP: Prepared as described in Examples 2 and 5.

Arp2/3 Complex: Purified as described in Example 1.

Antifoam: Sigma antifoam

B. Concentration of Stock Reagents and Assay Composition

Arp2/3-Mediated Actin Polymerization Protocol

| | | | Assay | |
|---|---|---|---|---|
| Reagents | Concentration | | Conc: | Unit |
| Actin | 0.8 | mg/ml | 3.41 | μM |
| Acrylodan-actin or | | mg/ml | | μM |
| Pyrene-actin | 1.5 | mg/ml | 0.55 | μM |
| GST-Cdc42 | 4.6 | mg/ml | 0.121 | μM |
| GST-105WASP | 0.2 | mg/ml | 0.044 | μM |
| Arp2/3 | 0.3 | mg/ml | 6.6 | μM |
| EGTA | 10 | mM | 55 | μM |

| | | | |
|---|---|---|---|
| Antifoam | 2% | 22 | PPM |

| | |
|---|---|
| Number of plates | 35.00 |
| Total Amount Needed | 397.00 |

First Step: Incubate Cdc42 with GTP

| | | | |
|---|---|---|---|
| Thaw appropriate amount ˜ | | 588 | μl |
| and add GTP | | 65.3224638 | μl |
| G-Buffer Total | | 265 | mls |
| 10X G Buffer | | 27 | mls |
| ATP | | 32 | mgs |
| DTT | | 133 | μL |
| Water | | 239 | mls |
| Actin Mix (Mix 1) | Vol: | 223.5 | mls |
| G-Buffer | | 135.95 | mls |
| Actin | | 80.02 | mls |
| Acyrlodan-actin or | | | mls |
| Pyrene-actin | | 6.88 | mls |
| GST-Cdc42 | | 587.90 | μL |
| Antifoam | | 49.17 | μL |
| Arp2/3 Mix (Mix 2) | Vol: | 173.50 | mls |
| G-Buffer | | 130 | mls |
| GST-105WASP | | 5344 | μL |
| Arp2/3 | | 1985 | μL |
| Antifoam | | 38 | μL |
| EGTA | | 1909 | μL |
| 10X Polymerization Salts | | 35 | mls |

Samples containing candidate agents (individually or as mixtures) are placed into wells on a multi-well plate. Mix 1 is added to each of the wells and mixed with the candidate agent. A sample of Mix 2 is then introduced into each well and the resulting mixture thoroughly mixed. Typically, Mix 1 and Mix 2 are mixed in 1:1 ratio (e.g., 50 μl each of Mix 1 and Mix 2).

Actin polymerization is measured as a function of time by exciting pyrene at 365 nm and by detecting an increase in fluorescence emission at 407 nm, or by exciting acrylodan at 405 nm and by detecting an increase in fluorescence emission at 460 nm. The change in fluorescence over time is utilized to determine a fluorescence parameter (e.g., maximal velocity, time to half maximal fluorescence intensity, or area under the curve of a plot of fluorescence versus time; see e.g., FIGS. 5A-5F).

### EXAMPLE 8

### Actin Polymerization Assay Using Full Length WASP

Full length WASP was prepared as described in Examples 2 and 6. This protein was then used as a substitute GST-105WASP in methods that were otherwise identical to the methods described in Example 7.

### EXAMPLE 9

### Actin Polymerization Assay Using Full Length N-WASP

Full length N-WASP was prepared as described in Examples 3 and 6. This protein was then used as a substitute GST-105WASP in methods that were otherwise identical to the methods described in Example 7.

### EXAMPLE 10

### Evaluation of the Activity of Upstream Regulators on WASP and N-WASP Activity

A. Background

In this experiment, the in vitro pyrene-actin assay of the type described in Example 7 was utilized with full length human WASP and N-WASP to analyze the regulation of WASP and N-WASP by Cdc42, Rac1, RhoA, RhoC, Nck1, Nck2 and PIP₂.

B. Materials

Full length human WASP and N-WASP were TAP-tagged (Rigaut, et al. (1999) Nat. Biotechnology 17:1030, which is incorporated herein by reference in its entirety for all purposes) at the C-terminus (see, also Examples 2 and 3). The recombinant WASP and N-WASP were expressed in human 293 cells and then purified using a TAP-tag protocol as described in Example 6.

Arp2/3 was purified as described in Example 1.

Nck1, Nck2, Cdc42 and Rac1 were GST-tagged and purified as described in Examples 4 and 5 and then used in the assays.

C. Methods and Results

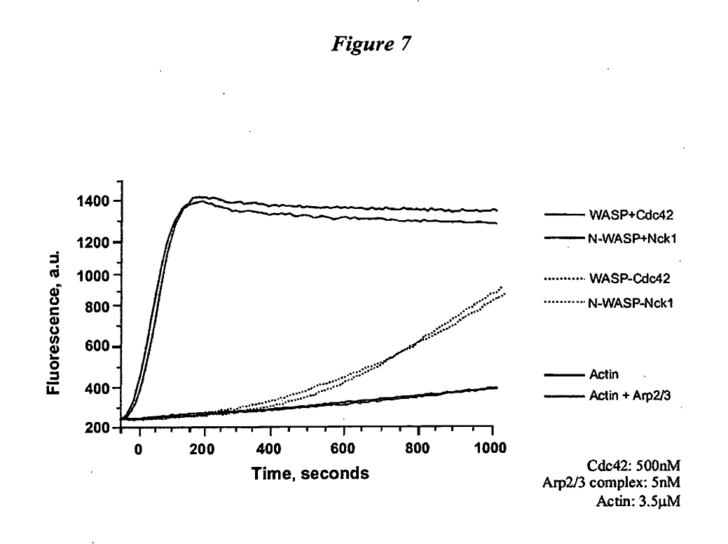

A first set of experiments were conducted to determine if full length WASP and N-WASP produced according to the methods described in Examples 2, 3 and 6 were regulated by upstream regulators such as Cdc42 and Nck1. Results are shown in FIG. 7. The activities shown in this plot illustrate: 1) that FL-WASP and N-WASP by themselves could only weakly stimulate actin polymerization; and 2) that the upstream regulators or activators Cdc42 or Nck1 accelerated actin polymerization 13-fold. That FL-WASP and N-WASP are regulated in a manner consistent with naturally occurring WASP and N-WASP indicates that the proteins produced by the methods provided herein are properly folded.

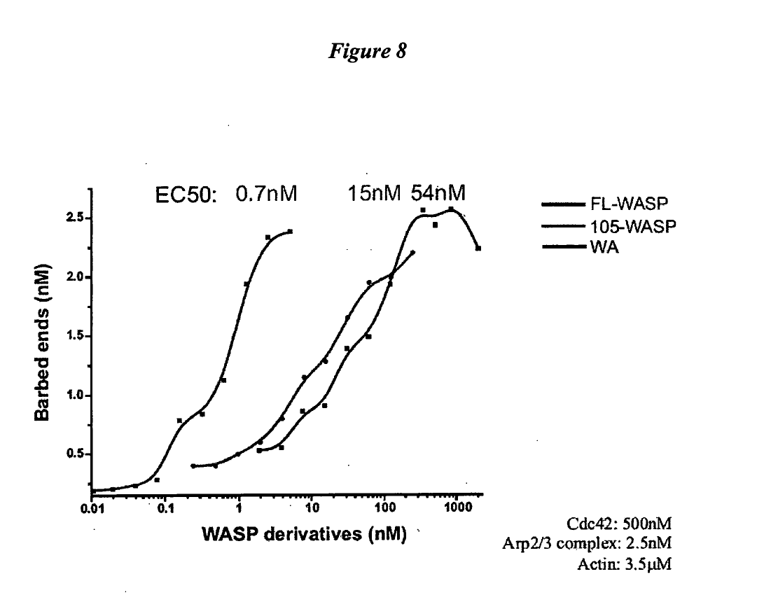

In a second set of experiments, the ability of various truncated forms of WASP were compared to the activity of the full-length protein. The polymerization assays were conducted in the presence of 500 nM Cdc42, 2.5 nm purified Arp2/3 complex and 3.5 μM actin. FL-WASP, 105 WASP and the VCA (see Example 2) domain were tested. The results of these trials were plotted to obtain EC50 values. The results are provided in FIG. 8 and in the chart below. These results demonstrate: 1) that at 3 nM, FL-WASP stimulated production of maximal concentration of barbed ends; 2) that FL-WASP was approximately 20 times more active than 105 WASP, which lacks the WH1 domain; and 3) that FL-WASP was more than 70 times more potent than the VCA/WA domain.

| | WASP | | | N-WASP | | |
|---|---|---|---|---|---|---|
| | Barbed ends, | Barbed ends | EC50, | Barbed ends, | Barbed ends | EC50, |
| Activator | nM* | % of max.** | nM | nM* | % of max.** | nM |
| Cdc42 | 3.8 | 87 | 16 | 1.3 | 9 | 287 |
| Rac1 | 1.4 | 12 | 80 | 1.9 | 28 | 31 |
| Nck1 | 4.0 | 94 | 10 | 3.4 | 75 | 11 |
| Nck2 | 2.6 | 50 | 12 | 3.5 | 78 | 7 |

*Total concentration of Arp2/3 complex in the assay is 4.2 nM
**After subtracting baseline (WASP without activators)

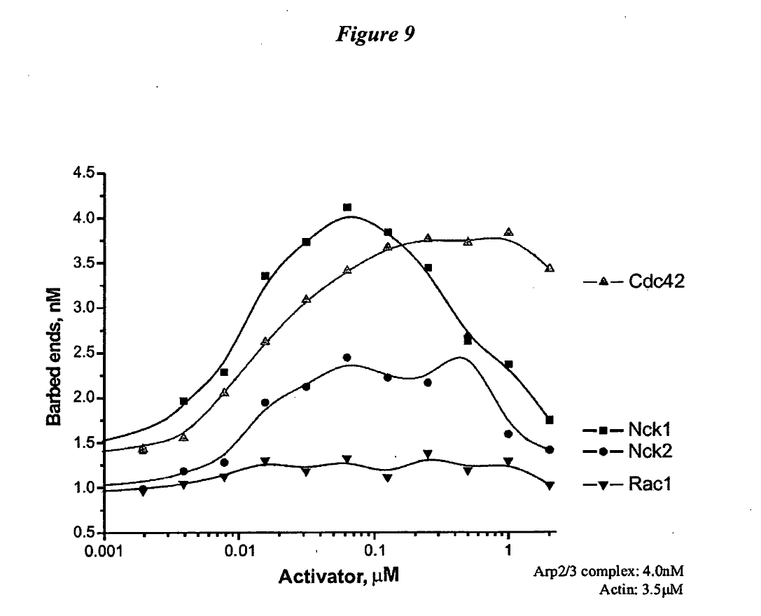

The ability of the upstream regulators Cdc42, Nck1, Nck2, and Rac1 to activate WASP was examined in a third experiment. The Arp2/3 complex in these experiments was 4.0 nM and the actin concentration 3.5 μM. The results are depicted in FIG. 9, which shows: 1) that Nck1 was the most potent of the activators tested; 2) that Cdc42 in the absence of Cdc42 can fully activate FL-WASP, and 3) that there is a bell shaped dependence between Nck1 and Nck2 and barbed end concentrations.

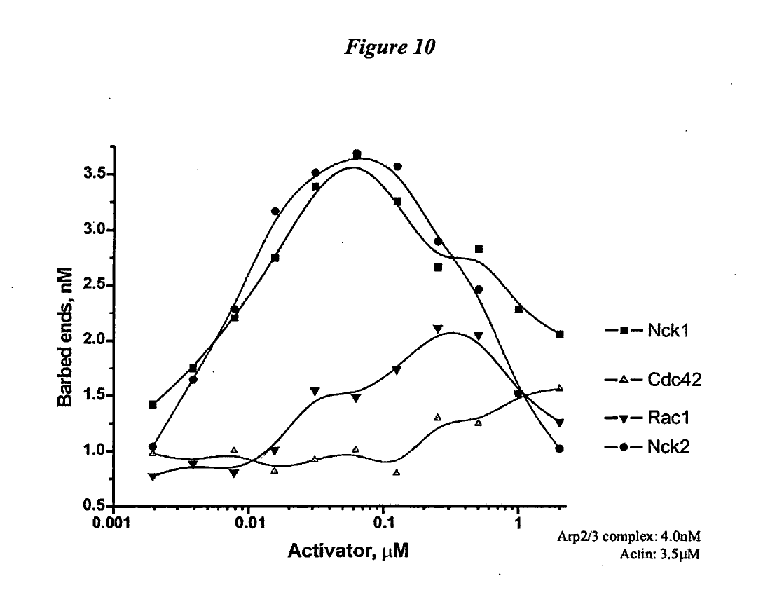

A fourth experiment similar to the third was conducted to ascertain the effect of Nck1, Nck2, Cdc42 and Rac1 on activation of FL-N-WASP. Arp2/3 and actin concentrations were as described for the third experiment. FIG. 10 summarizes the results in graphical form and shows that: 1) Rac1 can activate FL-N-WASP; 2) Rac 1 was a more potent N-WASP activator than Cdc42 in the absence of PIP₂ vesicles; 3) Nck1 and Nck2 were the only activators tested that can stimulate production of maximal concentration of barbed ends; 4) Nck2 is a significantly better activator of N-WASP than WASP; and 5) there is a bell shaped dose dependence for Nck1, Nck2 and Rac1.

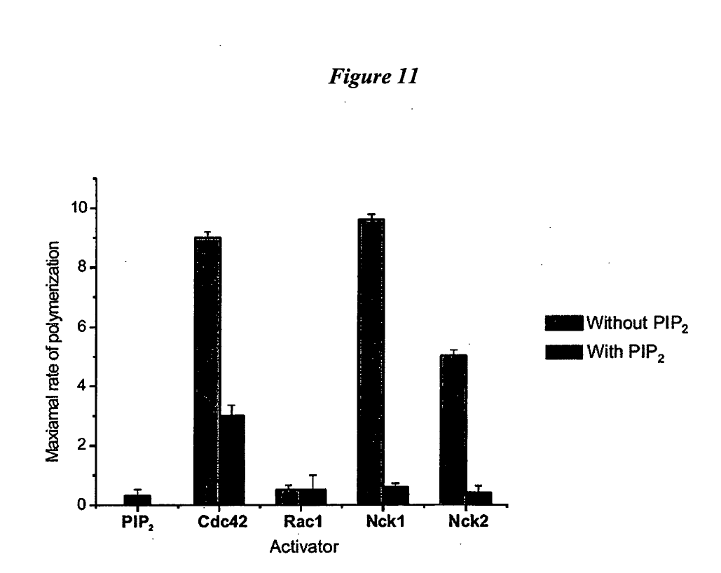

The effect of PIP₂ on the ability of upstream regulators to regulate FL-WASP was evaluated in a fifth set of experiments. The results are depicted in graphical format in FIG. 11. This figure indicates: 1) that PIP₂ had minimal, if any, effect of FL-WASP in the absence of small GTPases or Nck; and 2) that PIP₂ had a strong inhibitory effect on WASP stimulated actin polymerization in the presence of both small GTPases or Nck.

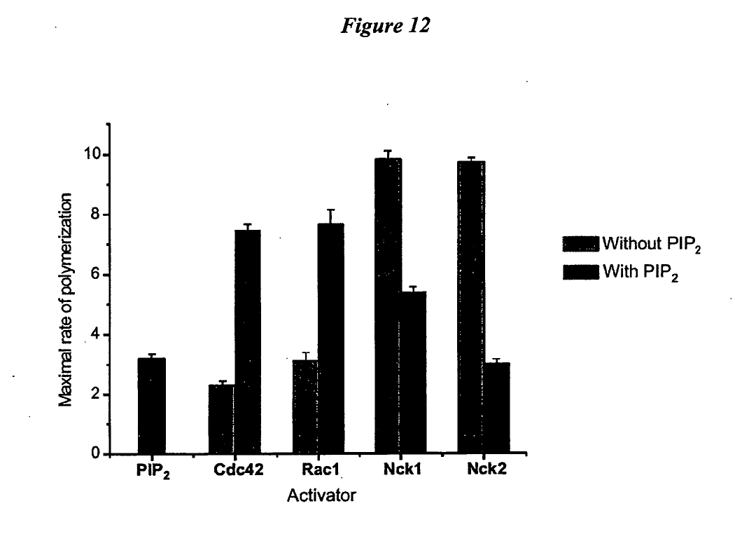

Another set of experiments similar to the fifth set were conducted using FL-N-WASP. These results are shown in FIG. 12 and indicate: 1) that PIP₂ had a marked synergistic effect on N-WASP activation by Rac1 or Cdc42; and 2) PIP₂ inhibited Nck stimulated activation of N-WASP.

D. Conclusions

Some of the conclusions that can be drawn from the foregoing results are as follows:
1. Highly active and regulated recombinant FL-WASP and N-WASP can be purified using the methods provided herein (see Examples 2, 4 and 6);
2. FL-WASP was a more potent Arp2/3 complex activator than certain truncated derivatives such as 105WASP and VCA.
3. Nck1 and Nck2 were the most powerful activators of FL-WASP and FL-N-WASP of the upstream regulatory proteins that were tested, as they stimulated generation of the maximal number of barbed ends.
4. Rac1 was a more potent FL-N-WASP activator than Cdc42.
5. Cdc42 was more effective on WASP-stimulated actin nucleation by Arp2/3 complex than on N-WASP-stimulated actin nucleation.
6. At higher concentrations, Nck1, Nck2 and Rac1 inhibited WASP- and N-WASP-stimulated actin polymerization.
7. Lipid vesicles containing PIP₂ significantly improved actin nucleation by Arp2/3 complex and N-WASP in the presence of either of the small GTPases. In contrast, the vesicles had only a modest effect on WASP stimulated actin nucleation in the presence or absence of the GTPases.
8. PIP₂ had a strong inhibitory effect on WASP-stimulated actin polymerization.
9. PIP₂ had either a synergistically or an inhibitory effect on N-WASP activation by small GTPases or Nck, respectively.
10. In contrast to Rac1 and Cdc42, RhoA and RhoC could not activate either of the WASP family members.

Collectively, the results demonstrate that differential regulation of WASP and N-WASP by cellular activators reflects fundamental differences at the protein-protein level, and indicate that there are previously unrecognized regulatory interactions.

### EXAMPLE 11

### Cloning of Candida albicans Formin Proteins

Standard molecular biological techniques, basically those described above in Examples 2-4, were used to construct E. coli plasmids expressing different fragments of the Candidia albicans formin FOR1. Two fragments were cloned: one spanning M972 to K1598 of C. albicans FOR1 and containing the FH1 and FH2 domains (SEQ ID NO:47), and the other spanning A1127 to K1598 of C. albicans FOR1 and containing the FH2 domain only (SEQ ID NO:48). Gateway expression vectors were used. The appropriate fragments were amplified from cDNA and Topo-cloned into pENTR/SD (Invitrogen), followed by recombination into the appropriate expression vector backbone (pDEST14 or pDEST15). Each fragment was either N-terminally tagged with GST followed by a TEV cleavage site, or C-terminally tagged with His6. The TEV cleavage site and the His6 tag were generated by incorporating their corresponding nucleotide sequences into the primers used to clone the C. albicans FOR1 fragments. All CTG codons (5 in the fragment containing the FH1 and FH2 domains and 1 in the fragment containing the FH2 domain) were mutagenized to TCG to address the non-conventional usage of this codon in C. albicans. The table below summarizes the details of the expression constructs made:

| Vector | | C. albicans FOR1 |
|---|---|---|
| backbone | Tag | fragment expressed |
| pDEST14 | His6, C-terminal | FH1-FH2 |
| pDEST14 | His6, C-terminal | FH2 |
| pDEST15 | GST-TEV, N-terminal | FH1-FH2 |
| pDEST15 | GST-TEV, N-terminal | FH2 |

### EXAMPLE 12

### Protein Production and Purification of Candida albicans Formin Domain Fusions

The C. albicans formin constructs described above were evaluated for their ability to produce protein. Competent cells BL21DE3 star were inoculated. Briefly, cells were grown with shaking at 37° C. until the OD reached 0.8. At that point, 0.25 mM isopropylthiogalactoside (IPTG) was added and incubation with shaking was continued overnight at RT. Cells were harvested by spinning in a Beckman centrifuge at 5 Krpm in a JLA 10 rotor for 30 minutes at 4° C. Cell pellets were resuspended in lysis buffer (50 mM Tris pH 8.0, 50 mM KCl, 1 mM DTT, 1 mM MgCl, 3% glycerol, plus protease inhibitor: 1 tablet/50 ml). Cells were lysed with the Microfluidizer by running 2 passes, 7-8 cycles each at 80 psi. Crude extract was cleared after spinning in a 45Ti rotor at 35 Krpm for 30 minutes at 4° C.

The formins were purified on either Ni-NTA column (His6 tagged proteins) or Gluthatione-Sepharose (GST tagged proteins). Resins were equilibrated with lysis buffer. Clear crude extracts were loaded onto column, columns were washed with lysis buffer followed by washing buffer (50 mM Tris pH 8.0, 50 mM KCl, 1 mM DTT, 1 mM MgCl, 3% glycerol), and proteins were eluted with either 300 mM imidazole (His6-tagged proteins) or 10 mM glutathione (GS-tagged proteins). Eluted proteins were analyzed by SDS PAGE (4-20%) (Invitrogen, San Diego Calif.), fractions were pooled and protein concentrations were measured by Coomassie Plus (Bradford)(Bio-Rad, Hercules, Calif.). Purified proteins were “drop-frozen” in liquid nitrogen and stored at −80° C.

### EXAMPLE 13

### Actin Polymerization Protocols Using Candida albicans Formin Fusion Proteins

A. Nucleation Activity of Candida albicans Formin Fusion Proteins

G-actin and pyrene-actin were prepared as described in Example 7.

Activity of purified formins from the constructs described above in Example 12 was assessed in an actin/pyrene-actin polymerization assay. Polymerization was carried out in G-buffer and was initiated by addition of 10× polymerization mix.

G-Buffer:

| | Final Concentration |
|---|---|
| Tris | 2 mM |
| CaCl₂ | 0.2 mM |
| Sodium Azide | 0.005% (w/v) |
| ATP | 0.2 mM |
| DTT | 0.5 mM |

Polymerization Mix:

| | Final Concentration |
|---|---|
| KCl | 400 mM |
| MgCl₂ | 8 mM |
| ATP | 0.8 mM |
| EGTA | 0.05 mM |

Gel filtered actin and pyrene-actin were diluted in G-buffer to the indicated final concentrations. Either purified C. albicans formin domains or formin domain-enriched fractions were added together with polymerization mix to start actin polymerization. Formin nucleated actin polymerization was monitored on a Gemini plate reader (excitation at 365 nm and emission at 407 nm). Protein components in the actin polymerization assay are provided below.

| Protein | Final Concentration | |
|---|---|---|
| Actin | 3.1 | μM |
| Pyrene Actin | 0.5 | μM |
| Formin | 0-500 | nM |

1. Formin FH1-FH2 with His6 Tag

Overexpression and purification of His6 tagged C. albicans FH1-FH2 domain yielded a fraction of highly enriched, but moderately pure protein. Ten milligrams of the protein was purified from 1 liter of bacteria. The protein was stable at −80° C. for several months. The His6 tagged C. albicans FH1-FH2 domain was shown to be active, i.e., nucleates actin polymerization, although rather high concentrations of the formin were used to observe saturation (300-500 nM).

2. Formin FH1-FH2 with GST Tag

The GST tagged C. albicans FH1-FH2 domain has shown the best biochemical properties in terms of solubility, stability and activity. Formin eluted from the gluthatione resin was highly enriched, but an additional purification step was performed. After a short lag phase (less than 100 s), 100 nM of GST tagged C. albicans FH1-FH2 domain nucleated actin polymerization, reaching saturation phase after 400s.

Further purification on SP Sepharose improved purity significantly. GST tagged C. albicans FH1-FH2 domain eluted from SP Sepharose was approximately 3-fold more active than the formin purified in the single step purification. Six milligrams of the protein were purified from 1 liter of bacteria using single step purification. After two steps of purification ˜2 mg of the protein were collected. The protein was stable at −80° C. for more than 6 months.

3. Formin FH2 with GST Tag

Overexpression and purification of GST tagged C. albicans FH2 domain yielded a fraction of highly enriched protein. Five milligrams of the protein was purified from 1 liter of bacteria. The protein is stable at −80° C. for several months. In terms of activity, this protein showed similar characteristics to the GST tagged C. albicans FH1—FH2 domain.

B. Formin Acrylodan-Actin Polymerization Protocol

In this actin polymerization assay using a formin as the actin nucleator, positive hits from the initial in vitro screening assay are tested for effects on actin polymerization in the absence of Arp2/3, an NPF, and an upstream regulator. A positive hit in this assay would indicate that the candidate agent affects actin polymerization directly, with the others possibly affecting the Arp2/3 complex, the NPF or the upstream regulator.

G-actin and acrylodan-actin were prepared as described in Example 7. The protein reagents in the assay are as follows:

| Protein | Final Concentration | |
|---|---|---|
| Actin | 1.8 | μM |
| Acryolodan-Actin | 0.2 | μM |
| Formin (GST | 0-500 | nM |
| tagged FH1-FH2) | | |

The Actin (Mix 1) and Formin (Mix 2) mixes are as follows:

| | 4.00 mL Total |
|---|---|
| Actin (Mix 1) | |
| G-Buffer | 2.947 mL |
| Actin | 0.96 mL (stock 0.63 mg/mL) |
| Acrylodan-Actin | 0.084 mL (stock 0.8 mg/mL) |
| Antifoam 2% | 8.8 μL |
| Formin (Mix 2) | |
| G-Buffer | 3.008 mL |
| 10X Polymerization Mix | 0.8 mL |
| Formin (GST-tagged | 0.183 mL (stock 0.175 mg/mL) |
| FH1-FH2) | |
| Antfoam 2% | 8.8 μl |

The assay protocol is as follows:
1. Place a 500 ml glass bottle (labeled as Mix 1) on ice.
2. Thaw ATP (100 mM), GTP (100 mM) and DTT (1 M).

3. Prepare fresh 1× Gi-buffer (pH 7.55+/−0.05, adjusted with 1 M HCl) at RT.

| 1X Gi-Buffer | 250 mL Total |
|---|---|
| 100X Gi-Buffer | 2.5 mL |
| ATP | 0.5 mL (stock 100 mM) |
| DTT | 0.125 mL (stock 1 M) |

4. Transfer 1× Gi-Buffer to Mix 1 bottle and keep it on ice.

5. Prepare fresh 10× Polymerization mix and keep it at RT.

| 10X Polymerization Mix | 5 mL Total |
|---|---|
| KCl | 0.67 mL (stock 3 M) |
| MgCl₂ | 0.08 mL (stock 1 M) |
| ATP | 0.04 mL (stock 100 mM) |
| EGTA | 0.01 mL (stock 250 mM) |
| Gi-Buffer | 4.2 mL |

6. Thaw the formin, actin and acrylodan-actin in a water bath, and then keep them on ice.
7. Prepare Mix 2 in a 500 mL bottle by adding the 1× Gi-buffer, 10× polymerization mix and formin. Add the antifoam and keep the mix at RT.
8. Add the actin and acrylodan-actin to ice-cold Mix 1. Add the antifoam and keep the mix on ice.
9. Add to each well of the plate 50 μL of Mix 1 and 50 μL of Mix 2.
10. Monitor formin nucleated actin polymerization on a Gemini plate reader by exciting acrylodan at 410 nm and detecting an increase in fluorescence emission at 450 nm.

### EXAMPLE 14

### Macrophage Podosome Formation Assay

A. Background

In this assay, positive hits from the in vitro screening assays are tested for effects on macrophage podosome formation. Macrophages express Arp2/3 and WASP, the latter of which is required for podosome formation (see, e.g., Linder, S. et al. (1999) Proc. Natl. Acad. Sci. USA 96:9648-9653). A cell-based secondary assay of this type uses the human monocytic cell ine THP-1, which undergoes macrophage differentiation and podosome formation in response to exposure to the phorbol ester, phorbol-12-myristate-13-acetate (PMA). A nuclear stain and an actin stain are used to quantify the number of podosomes/cell in a culture of differentiated THP-1 cells in the absence or presence of positive hits from the in vitro screening assays.

B. Materials and Methods

Human monocytic THP-1 cells (ATCC Accession No. TIB-202) were grown in RPMI containing 10% FCS and 2 mM L-glutamine. THP-1 cells, at 5×10⁶ cells/100 mm plate or 1.5×10⁷ cells/150 mm plate were differentiated in vitro by exposure to 50 nM PMA (Sigma-Aldrich-Fluka, p8139) for 48 hours at 37° C. in 5% CO₂. After incubation, the media was removed, the cells were washed once with PBS, and treated with trypsin to dislodge the flattened, differentiated cells from the plate. After the cells were dislodged, the trypsin was neutralized with media, and the cells were pelleted at 1000×g for 5 min in a tabletop centrifuge, and resuspended at 5×10⁵/ml in RPMI containing 10% FBS.

Differentiated THP-1 cells were plated at 50,000 cells/well in 100 μl in Nunc dark wall, glass 96-well plates (VWR Scientific, 73520-174) and incubated for 2 hours at 37° C. After incubation, 100 μl of a 2× concentration of test compound or positive control (diluted in THP-1 media) or cell media control was added. After incubation for 15 to 60 min at 37° C., 60 μl 10% formaldehyde solution was added to fix the cells. After incubation for 15 min at RT, the formaldehyde solution was removed, and 100 μl 0.1% Triton-X/PBS was added to each well to permeabize the cells for 15 min at RT.

Podosomes in the THP-1 cells were visualized using a nuclear stain and an actin stain as follows. A 100 μl of a 1:100 dilution of Alexa 568 Phalloidin (Molecular Probes Inc, A-12380) and 1:10,000 dilution of DAPI (Sigma-Aldrich-Fluka, D9542, 10 mg/ml in PBS) were added to each well and incubated for 15 min at RT. The stains were removed and 150 μl PBS was added to each well. The podosomes appeared as actin-rich dots on the underside of the THP-1 cells. The DAPI and TRITC channels were imaged with an Axon microscope system at 20× magnification (Molecular Devices, Inc.). Podosomes were best viewed +5 μm from the focus plane. The fraction of THP-1 cells positive for podosomes was quantified using Image Express software (Molecular Devices, Inc.).

C. Results and Discussion

In this assay, the THP-1 cells are visualized by the nuclear stain and podosomes are visualized by the actin stain. The effect of a modulator of podosome formation is measured by counting the frequency of THP-1 cells with podosomes, viewed as actin-rich dots on the underside of the attached cells. In this assay, the positive control, the microtubule poision nocodazole, as well as a variety of Arp2/3 inhibitors, which were identified as actin polymerization inhibitors in the high throughput screening assay and characterized as Arp2/3 inhibitors in the in vitro secondary assays, prevented podosome formation in PMA-treated THP-1 cells in a dose-dependent manner, as illustrated in FIG. 13.

### EXAMPLE 15

### Listeria monocytogenes Motility Assay

A. Background

In this assay, positive hits from the in vitro screening assays are tested for effects on Listeria motility. Pathogenic bacteria such as Listeria monocytogenes use the host cell actin machinery for motility and spread of infection (see, e.g., Robbins, J. R. et al. (1999) J. Cell Biol. 146:1333-1349). The bacterial surface protein ActA directly activates Arp2/3, which is required for actin polymerization and Listeria motility. A cell-based secondary assay of this type uses Listeria monocytogenes and SKOV-3 human ovarian cancer cells. An anti-Listeria antibody and an actin stain are used to quantify Listeria motility in a culture of SKOV-3 cells in the absence or presence of positive hits from the in vitro screening assays.

B. Materials and Methods

SKOV3 cells (ATCC Accession No. HTB-77) were seeded into dark-wall 96-well plates (Falcon) at 9333 cells/well in 100 PI RPMI containing 5% FBS (without antibiotics). The cells were incubated O/N at 37° C. in 5% CO₂. A culture of Listeria monocytogenes (ATCC Accession No. 984) was grown in brain heart infusion (Difco Inc., DF0037-15) on a rotary mixer at 37° C. The O/N culture was diluted 1:100 with brain heart infusion and incubated with shaking for 3 hr at 37° C.

The SKOV3 cells were infected with 0.2 μl (or 10 μl of 1:50 dilution in SKOV3 media) per well of the 96-well plate After incubation for 90 min at 37° C., 110 μl of a 2× concentration of test compound or positive control, diluted in cell media (RPMI/5% FBS), or cell media control was added. After a further incubation for 60 min at 37° C., the infected cells were fixed with 60 μl of 10% formaldehyde. After incubation for 15 min at RT, the media was removed, and the fixed, infected cells were permeabilized with 100 μl 0.1% Triton-X in PBS. After 15 minutes at RT, the Triton-X/PBS solution was removed.

Listeria cells were visualized using a labeled antibody, and Listeria and actin were stained as follows. A 1:200 dilution (in PBS) of Listeria pAb (Rabbit, U.S. Bio: L2650-01A) was added to the permeabilized infected cells. After incubation for 60 min at RT, the antibody solution was removed, and a 1:200 dilution of Alexa Fluor 568 Phalloidin (Molecular Probes Inc., A-12380) and a 1:400 dilution of Alexa Fluor 488 goat anti-rabbit secondary antibody (Molecular Probes Inc, A-11008) were added. After incubation for 60 min at RT, the actin stain and secondary antibody solution were removed and 150 μl PBS was added to each well. The FITC and TRITC channels were imaged with an Axon microscope system (Molecular Devices, Inc.) and the fraction of Listeria cells positive for actin staining was quantified using Image Express software (Molecular Devices, Inc.).

C. Results and Conclusions

In this assay, Listeria is visualized by the secondary antibody and actin is visualized by the actin stain. The effect of a modulator of Listeria motility is measured by scoring the fraction of Listera cells associated with actin. In this assay, several Arp2/3 inhibitors, which were identified as actin polymerization inhibitors in the high throughput screening assay and characterized as Arp2/3 inhibitors in the in vitro secondary assays, prevented Listeria motility in a dose-dependent manner, as illustrated in FIG. 14.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purpose.

TABLE 1

| | | SEQ | | | | | | |
|---|---|---|---|---|---|---|---|---|
| | | ID | | | | | | |
| All members | Accession # | NO. | Lamellipodia | Filopodia | Phagocytosis | Endocytosis | Pathogens | |
| Actin | P60709 | | Actin | Actin | Actin | Actin | Actin | |
| Arp2/3 complex | O15142 (Arp2) | | Arp2/3 | Arp2/3 complex | Arp2/3 complex | Arp2/3 | Arp2/3 complex | Ubiquitous |
| | | | complex | | | complex | | |
| Formins | Q9Y613 | | | | | | | |
| VASP | P50552 | | | VASP | | | VASP | Invasion |
| Ena | | | | | | | | |
| Mena | | | | | | | | |
| WASP | P42768 | | WASP | | | | | Inflammation |
| | | | and | | | | | |
| | | | N-WASP | | | | | |
| N-WASP | O00401 | | | N-WASP | | N-WASP | N-WASP | |
| WAVE1, 2 and 3 | Q92558, | | WAVE1 &2 | Wave2 | | | | |
| | Q9Y6W5 & | | | | | | | |
| | Q9UPY6 | | | | | | | |
| ActA - Listeria | | | | | | | ActA | |
| Cdc42 | P21181 | | | Cdc42 | Cdc42 | | | |
| TCL & TC10 | Q9H4E5 P17081 | | | | | | | |
| Rac1 | P15154 | | Rac1 | | Rac1 | Rac1 | | |
| RhoA | P06749, P08134 | | | | RhoA | | | Invasion |
| and RhoC | | | | | | | | |
| IRS53 | BAC57946 | | | IRS53 | | | | |
| PAK1 | Q13153 | | | | | | | |
| PIP2 | | | | | | | | |
| Nck1 &2 | P16333 & | | Nck1 | | | | Nck | |
| | O43639 | | | | | | | |
| Grb2 | P29354 | | | | | | | |
| Btk/Itk | | | | | | | WIP | |
| WIP | O43516 | | | | | | | |
| WICH | JC7807 | | | | | | | |
| IcsA | CAC05837 | | | | | | IcsA | |
| Src kinases | P12931 | | Src kinases | Src kinases | | | | |
| Hck | P08631 | | Hck | Hck | | | | |
| Fyn | P06241 | | Fyn | Fyn | | | | Neurite extension |
| CARMIL/ | AAK72255 | | | | | | | Chemotaxis |
| Acan125 | | | | | | | | |
| PIR121 | | | | PIR121 | | | | |
| Nap125 | | | | Nap125 | | | | |
| HSPC300 | AAF28978 | | | HSPC300 | | | | |
| EPLIN - inhibitor | | | | | | | | |
| IRS53 | | | | | | | | |
| Intersectin | Q15811 | | | | | Intersectin-2 | | |
| Cofilin | P23528 | | Cofilin | | | | | Chemotaxis |
| ProFilin | P07737 | | Profilin | Profilin | | | | |
| Gelsolin | P06396 | | | | | | | |
| CapZ | P52907 | | CapZ | | | | | |
| Vita. D | P02774 | | | | | | | |
| binding prot. | | | | | | | | |
| Coronin | Q92828 | | | | | | | |
| Fascin | Q16658 | | | Fascin | | | | Invasion |
| Mysoin-X | | | | Mysoin-X | | | | |
| Dynamin | Q05193 | | | | | Dynamin | | |
| PSTPIPI | | | | | | | | |
| CD2AP | | | | | | | | |
| CIP4 | | | | | | | | |

TABLE 2

| | | Nucleation | | |
|---|---|---|---|---|
| | Actin | Promoting | Upstream | Actin Binding |
| Actin Type | Nucleators | Factors | Regulators | Proteins |
| G-actin | Arp2/3 complex | WASP | Cdc42 | Cofilin |
| Acrylodan-G- | Formins | N-WASP | TCL & TC10 | Profilin |
| actin | | | | |
| Pyrene-G-actin | VASP | WAVE1, 2 and 3 | Rac1 | Gelsolin |
| | Ena | ActA - Listeria | RhoA and RhoC | CapZ |
| | Mena | | IRS53 | Vitamin D |
| | | | | binding prot. |
| | | | PAK | Coronin |
| | | | PIP2 | Fascin |
| | | | Nck | |
| | | | Grb2 | |
| | | | Btk/Itk | |
| | | | WIP | |
| | | | WICH | |
| | | | IcsA | |
| | | | Src kinases | |
| | | | Hck | |
| | | | Fyn | |
| | | | CARMIL/Acan125 | |
| | | | PIR121 | |
| | | | Nap125 | |
| | | | HSPC300 | |
| | | | EPLIN - inhibitor | |
| | | | IRS53 | |
| | | | Intersectin | |

TABLE 3
Sequence information and approximate domain boundaries for exemplary nucleation promoting factors. Domain boundaries refer to
amino acids from the corresponding full-length amino acid sequence.

| | | SEQ ID | | | | | | | |
|---|---|---|---|---|---|---|---|---|---|
| | | NO: | SEQ ID | | | | | | |
| Nucleation | GenBank | (nucleic | NO: | | | | | VCA | |
| Promoting | Accession | acid | (protein | WH1 | B- | CRIB | PolyPro | Protein | |
| Factor | No. | sequence) | sequence) | Region | Domain | Domain | Sequence | Sequence | Reference |
| WASP | P42768 | 1 | 2 | 1-142 | 219-237 | 230-288 | 312-421 | 429-501 | Winter, et al. (1999) |
| | | | | | | | | | Curr. Biol. 9: 501-4; |
| | | | | | | | | | and Yarar D., et al. |
| | | | | | | | | | (1999) Curr. Biol. |
| | | | | | | | | | 9: 555-58 |
| N-WASP | O00401 | 3 | 4 | 1-154 | 181-200 | 192-250 | 274-392 | 393-501 | Rohatgi, et al. |
| | | | | | | | | | (1999) Cell 97: 221-31 |
| SCAR/WAVE1 | Q92558 | 37 | 38 | 1-168 | 171-225 | N/A | 275-492 | 492-559 | Welch, et al. (1998) |
| | | | | | | | | | Science 281: 105-108 |
| SCAR/WAVE2 | Q9Y6W5 | 39 | 40 | 1-168 | 168-202 | N/A | 265-400 | 431-498 | Machesky et al, |
| | | | | | | | | | Molecular Biology |
| | | | | | | | | | of the Cell, 14: 670-684, |
| | | | | | | | | | 2003 |
| SCAR/WAVE3 | Q9UPY6 | 41 | 42 | 1-168 | 168-202 | N/A | 251-436 | 436-502 | Machesky et al, |
| | | | | | | | | | Molecular Biology |
| | | | | | | | | | of the Cell, 14: 670-684, |
| | | | | | | | | | 2003 |
| ActA | | NA | | NA | NA | NA | NA | NA | Welch et al. (1998) |
| | | | | | | | | | Science 281: 105-8 |

TABLE 4

| | SEQ ID NO: | | | |
|---|---|---|---|---|
| WASP/N- | (exemplary | SEQ ID NO: | Activate | Regulated By Which |
| WASP Protein | nucleic acid) | (amino acid) | Arp2/3? | Upstream Regulators? |
| FL-WASP | 1 | 2 | Yes | Cdc42, PIP₂, Nck and Rac1 |
| FL-N-WASP | 3 | 4 | Yes | Cdc42, PIP₂, Nck, Rac1 |
| WASP VCA Domain | 5 | 6 | Yes | None |
| N-WASP VCA Domain | 7 | 8 | Yes | None |
| 105WASP | 9 | 10 | Yes | Cdc42, PIP₂, Nck and Rac1 |
| 98 N-WASP | 11 | 12 | Yes | Cdc42, PIP₂, Nck and Rac1 |
| Myc-WASP-TAP | 13 | 14 | Yes | Cdc42, PIP₂, Nck and Rac1 |
| Myc-N-WASP-TAP | 15 | 16 | Yes | Cdc42, PIP₂, Nck and Rac1 |
| GST-105WASP | 17 | 18 | Yes | Cdc42, PIP₂, Nck and Rac1 |
| Myc-105WASP-TAP | 19 | 20 | Yes | Cdc42, PIP₂, Nck and Rac1 |
| GST-tev-98N-WASP | 21 | 22 | Yes | Cdc42, PIP₂, Nck and Rac1 |
| Myc-98N-WASP-TAP | 23 | 24 | Yes | Cdc42, PIP₂, Nck and Rac1 |

What is claimed is:

1. A method for screening an agent for capacity to modulate the activity of a component involved in actin polymerization, the method comprising:
(a) combining actin polymerization assay components in the presence of a test agent, the components comprising (i) pyrene-G-actin (pyrene-globular actin) or acrylodan-G-actin (acrylodan-globular actin), (ii) an Arp2/3 complex, (iii) a nucleation promoting factor (NPF) protein that can initiate nucleation of actin, wherein the NPF protein is selected from the group consisting of a WASP protein, a N-WASP protein, a SCAR1/WAVE1 protein, a SCAR2/WAVE2 protein and a SCAR3/WAVE3 protein, and (iv) and an upstream regulator that can activate the NPF protein, wherein the upstream regulator is selected from the group consisting of a Cdc42 protein, a Rac1 protein, a Nck1 protein, a Nck2 protein and phosphatidylinositol-1,4-bisphosphate (PIP₂);
(b) detecting fluorescence over time to determine a fluorescence parameter that is a measure of the polymerization of pyrene-G-actin into pyrene-F-actin (pyrene-filamentous actin) or acrylodan-G-actin into acrylodan-F-actin (acrylodan-filamentous actin); and
(c) comparing the polymerization parameter determined in (b) with the polymerization parameter for a control reaction conducted in the absence of agent, wherein a difference is an indication that the agent is a modulator of the activity of one of the polymerization components.

2. The method of claim 1, wherein the components further comprise unlabeled actin, wherein the unlabeled actin is G-actin or G-actin plus F-actin seeds.

3. The method of claim 2, wherein combining comprises mixing a first and a second mixture, the first mixture containing G-actin, pyrene-G-actin or acrylodan-G-actin, and the upstream regulator protein, and the second mixture containing the NPF protein.

4. The method of claim 3, wherein the second mixture also contains the Arp2/3 complex.

5. The method of claim 3, wherein the first mixture also contains an antifoam agent and the second mixture also contains an antifoam agent and polymerization salts.

6. The method of claim 1, wherein the NPF protein is a WASP protein.

7. The method of claim 6, wherein the WASP protein comprises SEQ ID NO:2.

8. The method of claim 6, wherein the WASP protein is a fusion protein that comprises a WASP domain that can activate the nucleation initiation activity of Arp2/3 and a tag.

9. The method of claim 8, wherein the WASP fusion protein is a Myc-WASP fusion.

10. The method of claim 8, wherein the WASP fusion protein is a GST-105 WASP fusion that has the amino acid sequence of SEQ ID NO:18.

11. The method of claim 8, wherein the WASP fusion protein is a Myc-WASP-TAP fusion.

12. The method of claim 1, wherein the NPF is a N-WASP protein.

13. The method of claim 12, wherein the N-WASP protein comprises SEQ ID NO:4.

14. The method of claim 12, wherein the N-WASP protein is a fusion protein that comprises an N-WASP domain that can activate the nucleation initiation activity of Arp2/3 and a tag.

15. The method of claim 14, wherein the N-WASP fusion protein is a Myc-N-WASP fusion.

16. The method of claim 1, wherein the upstream regulator protein is a fusion protein that comprises a domain from Cdc42, Rac1, Nck1 or Nck2, wherein the domain can activate the NPF protein, and a tag.

17. The method of claim 16, wherein the fusion protein comprises the Cdc42 domain and the tag.

18. The method of claim 17, wherein the fusion protein is a GST-Cdc42 fusion.

19. The method of claim 16, wherein the fusion protein comprises the Rac1 domain and the tag.

20. The method of claim 19, wherein the fusion protein is a GST-Rac1 fusion.

21. The method of claim 16, wherein the fusion protein comprises the Nck1 domain and the tag.

22. The method of claim 21, wherein the fusion protein is a GST-Nck1 fusion.

23. The method of claim 16, wherein the fusion protein comprises the Nck2 domain and the tag.

24. The method of claim 23, wherein the fusion protein is a Nck2 fusion.

25. The method of claim 1, wherein the parameter is maximal velocity.

26. The method of claim 1, wherein the parameter is time to half the maximum fluorescence reading.

27. The method of claim 1, wherein the parameter is the slope from the time point at which a curve being quantified or its controls have undergone at least 10% of their total fluorescence change upon polymerization to the time point at which the reaction being quantified or its controls have undergone no greater than 90% of their total fluorescence change.

28. The method of claim 1, wherein the parameter is the area under a plot of fluorescence versus time.

29. The method of claim 1, wherein combining comprises mixing a sample containing the NPF and a sample containing the upstream regulator protein with the other components, and wherein the purity of each of the NPF protein and the upstream regulator protein in their respective samples is at least 90% by weight relative to other proteins.

30. The method of claim 29, wherein the purity of the NPF protein and the upstream regulator protein is at least 95%.

31. The method of claim 1, wherein the NPF comprises WASP or N-WASP modified to include a protein-binding motif or domain, and the upstream regulator comprises a protein that binds to the modified site in WASP or N-WASP and thereby modulates the activation of Arp2/3 by the modified WASP or N-WASP.

32. A method for screening an agent for capacity to modulate the activity of a component involved in actin polymerization, the method comprising:
(a) combining actin polymerization assay components in the presence of a test agent, the components comprising (i) unlabeled actin and (ii) pyrene-G-actin (pyrene-globular actin) or acrylodan-G-actin (acrylodan-globular actin), and optionally comprising (iii) an Arp2/3 complex, (iv) a formin that can initiate nucleation of actin, (v) a nucleation promoting factor (NPF) protein that can initiate nucleation of actin, wherein the NPF protein is selected from the group consisting of a WASP protein, a N-WASP protein, a SCAR1/WAVE1 protein, a SCAR2/WAVE2 protein and a SCAR3/WAVE3 protein, and (vi) and an upstream regulator that can activate the NPF protein, wherein the upstream regulator is selected from the group consisting of a Cdc42 protein, a Rac1 protein, a Nck1 protein, a Nck2 protein and phosphatidylinositol-1,4-bisphosphate (PIP₂);
(b) detecting fluorescence over time to determine a fluorescence parameter that is a measure of the polymerization of pyrene-G-actin into pyrene-F-actin (pyrene-filamentous actin) or acrylodan-G-actin into acrylodan-F-actin (acrylodan-filamentous actin); and
(c) comparing the polymerization parameter determined in (b) with the polymerization parameter for a control reaction conducted in the absence of agent, wherein a difference is an indication that the agent is a modulator of the activity of one of the polymerization components.

33. The method of claim 32, wherein the components comprise the Arp2/3 complex and the NPF, wherein the NPF is a constitutively active form or domain of the WASP protein or the N-WASP protein.

34. The method of claim 32, wherein the components comprise the formin.

35. A kit comprising: (i) a purified Arp2/3 complex; (ii) a purified nucleation promoting factor (NPF) protein selected from the group consisting of a WASP protein, a N-WASP protein, a SCAR1/WAVE1 protein, a SCAR2/WAVE2 protein and a SCAR3/WAVE3 protein; and (iii) a purified upstream regulator protein selected from the group consisting of a Cdc42 protein, a Rac1 protein, a Nck1 protein and a Nck2 protein.

36. The kit of claim 35, further comprising purified G-actin and pyrene-G-actin.

37. The kit of claim 35, further comprising purified G-actin and acrylodan-G-actin.

38. The kit of claim 36 or 37, further comprising polymerization salts.

39. A kit comprising: (i) a purified unlabeled actin and (ii) a pyrene-G-actin or an acrylodan-G-actin.

40. The kit of claim 39, further comprising a purified Arp2/3 complex and a purified nucleation factor (NPF) protein that can initiate nucleation of actin, wherein the NPF protein is selected from the group consisting of a WASP protein and a N-WASP protein, and wherein the NPF protein is a constitutively active form or domain of the WASP protein or the N-WASP protein.

41. The kit of claim 39, further comprising a formin that can initiate nucleation of actin.
